(19) United States
(12) Patent Application Publication
(10) Pub. No.: US 2024/0041922 A1
(43) Pub. Date: Feb. 8, 2024

(54) METHODS AND COMPOSITIONS

(71) Applicant: Monash University, Clayton, Victoria (AU)

(72) Inventors: Dhanushika RATNAYAKE, Clayton, Victoria (AU); Peter CURRIE, Clayton, Victoria (AU); Mikael MARTINO, Clayton, Victoria (AU)

(21) Appl. No.: 17/996,754

(22) PCT Filed: Apr. 19, 2021
(86) PCT No.: PCT/AU2021/050349

§ 371 Date: Oct. 20, 2022

(30) Foreign Application Priority Data
Apr. 20, 2020 (AU) 2020901237
Dec. 17, 2020 (AU) 2020904717

(51) Int. Cl. A61K 35/15; C12N 5/077; A61K 38/45; C07K 14/715; A61P 21/06

(52) U.S. Cl. CPC A61K 35/15; C12N 5/0658; A61K 38/45; C07K 14/7158; A61P 21/06; C12Y 204/01012

(57) ABSTRACT

Methods of stimulating muscle regeneration, comprising delivering to a muscle a CCR5 interacting agent or encoding molecule. The CCR5 interacting agent binds to muscle stem cells and stimulates myoblast proliferation and muscle regeneration. One example of the CCR5 interacting agent is NAMPT comprising a cytokine finger motif or a derivative thereof. Methods and compositions include cellular compositions, which expresses the CCR5 interacting agent; including a population of satellite or macrophage cells or their precursors/progeny and their applications in stem cell therapy or for use in treating a muscular deficiency, disorder or injury. The examples show muscle tissue regeneration with minimal fibrosis. Also enabled is a NAMPT polypeptide fragment comprising a C-terminal portion of NAMPT comprising a cytokine finger. Compositions further comprise the NAMPT polypeptide fragment and one or more of; tissue stem cell or macrophage or precursor/progeny, scaffold or a retentive material, tissue delivery enhancing or cell retention moiety.

### PRIORITY CLAIM TO RELATED APPLICATIONS

This application is a U.S. national stage filing under 35 U.S.C. § 371 from International Application No. PCT/AU2021/050349, filed on 19 Apr. 2021, and published as WO2021/212168 on 28 Oct. 2021, which claims the benefit under 35 U.S.C. 119 to Australian Application No. 2020901237, filed on 20 Apr. 2020, and to Australian Application No. 2020904717, filed on 17 Dec. 2020, the benefit of priority of each of which is claimed herein.

### FIELD OF DISCLOSURE

This disclosure relates to the technical field of productive tissue repair and regeneration and to the treatment of subjects in need of same.

### BACKGROUND ART

The reference in this specification to any prior publication, or to any matter which is known, is not, and should not be taken as an acknowledgment or admission or any form of suggestion that that prior publication or known matter forms part of the common general knowledge in the field of endeavour to which this specification relates.

Bibliographic details of documents referred to are listed at the end of the specification.

Skeletal muscle typically forms approximately 40% of a body mass in humans.

It is formed during development by the myogenesis wherein paired blocks of paraxial mesoderm known as somites give rise to the myotome that expands to form an integrated and complex musculature. Throughout life, growth, regeneration and repair of muscle tissue is driven by mesoderm derived skeletal muscle resident stem cells.

Skeletal muscle typically forms approximately 40% of a body mass in human adults. It is formed during development by myogenesis wherein paired blocks of paraxial mesoderm known as somites give rise to a transitory myotome that forms muscle stem cells and expands to form an integrated and complex musculature through fusion of myoblasts to the surface of myotubes. In a further stage of myogenesis, muscle stem cells (called satellite cells) migrate to occupy a niche between the sarcolemma and basal lamina of individual myofibers. Amniotes are born with a full set of muscle fibres and, in adults, muscle repair is generally effected through an increase in the size of existing fibres. Throughout life, homeostasis, growth, regeneration and repair of muscle tissue is driven by mesoderm derived skeletal muscle resident stem cells. At a molecular level, quiescent satellite cells require and express the transcription factor PAX7 and also express PAX3. Following skeletal muscle damage, some satellite cells become activated, proliferate to form myoblasts that differentiate and fuse to form new myofibres or merge with and repair damaged muscle fibres. This myogenic programme is governed by myogenic regulatory factors, MYF5, MYOD, MYOG and MRF4. A wealth of other factors and cells associated with the muscle niche are thought to be involved in the complex cellular processes and final production of functional tissue in homeostatic and regenerative contexts. Hence it has been difficult to date to identify the source and nature of the signals that stimulate satellite cell activation and proliferation.

The satellite cell is archetypal of a unipotent tissue-resident stem cell that occupies a specific anatomical niche within a differentiated tissue. Decades of research have revealed the extraordinary capacity of this system to effectively coordinate muscle repair in response to a wide variety of insults. Despite this demonstrated regenerative capacity, transplantation of isolated muscle stem cells has yet to provide therapeutic impact, and pro-regenerative treatments that stimulate muscle stem cells are entirely lacking at this juncture.

There is provided inter alia compositions for use in myoblast based therapy outcomes to address significant and diverse unmet clinical needs.

### SUMMARY OF THE DISCLOSURE

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−10%, or +/−5%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise.

The present disclosure is predicated, in part, on the results of experiments using time-lapse photography to describe at the single cell level the temporal and cellular framework for muscle regeneration. This the first time these interactions and pathways have been visualized in toto in a vertebrate system and it is proposed and enabled herein that the newly observed cellular behaviours and methods and compositions described herein can be reproduced in other tissue types.

A surprisingly direct and essential role for specific macrophage subsets has been identified in modulating tissue regeneration in vivo, demonstrating that a proportion of wound-attracted macrophages form a transient stem cell niche with resident tissue stem cells and induce their activation. Ablation of this niche-specific macrophage subset leads to a severe reduction in the number of proliferating progenitors present within the injury site, and a consequent regeneration deficit. The term injury herein relates broadly to any externally or internally inflicted or present wound where tissue regeneration is required to replace lost tissue or rebuild or regenerate functional tissue lost through any process such as a disease process, the results of infection or trauma, longevity, poor diet and lack of exercise, etc.

An obligate satellite cell-macrophage niche has been identified in real time and within the wound to instigate efficient skeletal muscle regeneration and injury repair. This demonstrates that a proportion of wound-attracted macrophages form a transient stem cell niche and are pro-myogenic. See FIG. 2 m where the larger fronded macrophage cell is seen in prolonged close contact with a smaller satellite cell that is seen to undergo division. Ablation of this niche-specific macrophage subset leads to a severe reduction in the number of myogenic progenitors present within the injury site, and a consequent muscle regeneration deficit.

In one embodiment, stem cells are tissue stem cells, such as a muscle stem cell. In one embodiment, the stem cells are skeletal muscle stem cells (satellite cells). Other muscle stem cells include heart tissue stem cells or non-striated muscle cells. As determined herein, in one embodiment, the tissue stem cell expresses a CCR5 receptor.

Accordingly, along with their well described ability to modulate pro-inflammatory and anti-inflammatory events, specific macrophage populations also provide a transient stem cell activating niche (the cells are spatially constrained together and interact directly in muscle tissue). Accordingly, macrophages or macrophage derived factors as detailed herein are proposed for use in modulating stem cell activity, in directly activating quiescent tissue stem cells and in tissue regeneration.

One macrophage derived factor described herein is Nicotinamide phosphoribosyltransferase (NAMPT, also known as visfatin and PBEF (pre-B cell enhancing factor). NAMPT is shown herein to be upregulated and produced by injury dwelling macrophages. Specific derivatives of NAMPT that interact with muscle stem cell receptor have been developed. Compositions comprising same have been determined (See Figures and Examples) to induce muscle stem cell proliferation and muscle regeneration. In one embodiment activation is via chemokine receptor binding. Other agents specifically acting through this receptor also stimulate muscle regeneration (e.g., for CCR5, CCL8 and CCL4). Importantly, treatment of muscle with NAMPT was associated with little or minimal fibrosis in a clinically relevant volumetric wound model. Accordingly, NAMPT and its functional derivatives are proposed for use in stimulating would healing and improving the quality of healing in order to promote full restoration of tissue function i.e., productive tissue repair and regeneration. Reference to NAMPT herein includes the full length molecule (e.g, SEQ ID No:4), functionally active parts or fragments (including more than one fragment arranged or operably connected to be functional) that activate quiescent tissue stem cells, and their derivatives comprising adaptations suitable for production, and clinical or commercial use, known in the art, such as enhanced tissue delivery or enhanced signalling functionalities. The term includes fusion proteins or conjugates comprising all or part of NAMPT that activate satellite cells as described herein. NAMPT includes orthologs and isoforms.

Reference to “functional derivatives” of NAMPT or NAMPTcif includes the full length molecule and parts, fragments of full length NAMPT or NAMPTcif (including orthogs and homologs), peptides in monomeric, dimeric, trimeric, teterameric or multimeric form, and variants and other modified forms thereof as further described herein, wherein the functional derivative, at least, binds quiescent tissue stem cells and induces signalling, activation and proliferation thereof. The present application provides for the use of know molecules such as NAMPT for a new use and also provides novel molecules including functional derivatives of known molecules for use in the presently disclosed compositions, methods or uses.

In one aspect, the present application provides compositions providing chemokine receptor interaction or binding activity or muscle tissue stem cell interacting activity for use in stimulating muscle regeneration. In one embodiment, the present application provides compositions providing chemokine receptor interaction or binding activity or satellite cell binding or interacting activity for use in stimulating muscle regeneration without fibrosis or substantially without fibrosis.

In one embodiment, the chemokine receptor is a CCR5 chemokine receptor or a tissue stem cell receptor that binds NAMPT, including a tissue stem cell receptor that binds NAMPTcif. In one embodiment the composition, comprising a cell or other agent that provides CCR5 interacting activity, binds to tissue stem cells, particularly muscle stem cells. The cell or agent may be modified to enhance the selectivity of binding to a specific tissue stem cell, as required and described herein. The cell or agent may be modified to enhance cytokine receptor signalling, as required and described herein.

Reference to “regeneration” in relation to a muscle is used herein in a broad context and includes the flow on effects on muscle and muscle associated tissue as a direct result of muscle stem cell (also called satellite cell) activation. Thus, regeneration includes muscle wound repair and muscle maintenance, growth, repair, augmentation of the ability of muscle cells to productively proliferate and form functional tissues. The term includes generation of muscle tissue, and repair of an injured muscle, and pertains to the process of muscle regeneration (myogenesis) commencing with activation and proliferation of muscle stem cells, proliferation of myoblasts, early differentiation into myocytes and terminal differentiation into myofibres. In one embodiment, regeneration is associated with minimal fibrosis which allows for establishment of native structures or regenerated tissue having normal or approximating normal biological properties rather than fibrotic or weakened tissue. Muscle functional properties may be determined by standard tests of contractile muscle function, including tests for strength (for example eccentric muscle contraction), power and endurance, as well as physical length and volume. The term also includes growth of muscle tissue in commercial cultures.

In one non-limiting embodiment, promoting muscle stem cell chemokine receptor signalling is particularly useful in treating subjects with a muscle injury including volumetric muscle loss injuries or muscle degeneration/atrophy, or muscular or neuromuscular impairments, muscular or neuromuscular degenerative conditions, myopathy, or the propensity therefore. In one embodiment, chemokine receptor binding activity is provided in the form of a cell such as a macrophage or stem cell expressing a chemokine receptor interacting/binding factor, and a molecule such as a polypeptide or peptide, a nucleic acid molecule, an antibody or a receptor interacting part thereof, a small molecule or other agent having or encoding chemokine receptor binding activity.

In one embodiment, the present application provides a method of stimulating proliferation of a stem cell, such as satellite cell proliferation. In one embodiment, the method comprises administering to a cell or subject an effective amount of a cellular composition comprising macrophages having chemokine receptor agonist activity. In one embodiment, the chemokine receptor agonist activity within one or more muscle stem cells stimulates or enhances muscle stem cell proliferation and induces muscle generation.

In one embodiment, the chemokine receptor is a CCR5 receptor.

In one embodiment, the CCR5 receptor is a tissue stem cell or tissue stem cell progeny CCR5 receptor. In one embodiment the CCR5 receptor is a satellite cell or satellite cell progeny CCR5 receptor.

In one embodiment, in vitro, in vivo and ex vivo applications are contemplated.

In one particular embodiment, the present application provides a method of stimulating muscle tissue regeneration, the method comprising administering to a muscle an effective amount of a composition comprising or encoding a CCR5 interacting agent, wherein the CCR5 interacting agent binds to muscle stem cells (satellite cells) and stimulates myoblast proliferation and muscle regeneration.

In one embodiment, the CCR5 interacting agent is a CCR5 agonist. That is, it stimulates receptor signalling or downstream events such as satellite cell activation and proliferation. In one embodiment, the CCR5 interacting agent specifically activates tissue stem cells. In one embodiment, the CCR5 interacting agent specifically activates satellite cells.

As described herein, in one embodiment, tissue regeneration stimulated by the method is associated with minimal fibrosis. Thus, in another aspect, the present application provides agents and methods of reducing fibrosis development in a patient or biological tissue subject to regenerative treatment.

In one embodiment, the present application provides a method suitable for regenerating muscle tissue in vitro, in vivo or ex vivo. Accordingly, CCR5 interacting agents described herein are proposed for use in stem cell based therapies and tissue engineering. In another embodiment, CCR5 interacting agents described herein are for use in artificial meat production in vitro.

In one embodiment, the CCR5 interacting agent competes with nicotinamide phosphoribosyltransferase (NAMPT) for interacting with muscle stem cells. NAMPT is a multi-functional protein which carries out a well-characterised enzymatic role in cellular metabolism and NAD regeneration when localised intracellularly. In addition, its secreted form (secreted NAMPT (secNAMPT)) has been documented to function as a cytokine with conflicting reports of regenerative, physiological and pathological functions in a number of tissues.

In one embodiment, the CCR5 interacting agent able to promote stem cell activation is secNAMPT or a part thereof comprising a cytokine finger (cif) motif or a derivative thereof comprising a cytokine finger (cif) motif. Reference herein to NAMPT includes reference to secNAMPT and derivatives, including parts or fragments, that are also CCR5 interacting molecules as described herein.

Accordingly, in one embodiment, the present application provides a method of stimulating tissue regeneration, the method comprising administering to a tissue an effective amount of a composition comprising or encoding secNAMPT or a part thereof comprising a cytokine finger (cif) motif or a derivative thereof comprising a cytokine finger (cif) motif, and optionally a tissue specific delivery moiety, wherein the secNAMPT, part or derivative binds to adult stem cells in the tissue and stimulates stem cell activation, proliferation and tissue regeneration.

In a further embodiment, the present application provides a method of stimulating muscle tissue regeneration, the method comprising administering to a muscle an effective amount of a composition comprising or encoding secNAMPT or a part thereof comprising a cytokine finger (cif) motif or a derivative thereof comprising a cytokine finger (cif) motif, wherein the secNAMPT, part or derivative binds to satellite cells and stimulates satellite cell activation, myoblast proliferation and muscle regeneration.

In one embodiment, the present application provides a method of stimulating muscle tissue regeneration, the method comprising administering to a muscle an effective amount of a composition comprising or encoding secNAMPT or a part thereof comprising a cytokine finger (cif) motif or a derivative thereof comprising a cytokine finger (cif) motif, wherein the secNAMPT, part or derivative binds to satellite cells and stimulates satellite cell activation, and myoblast proliferation and muscle regeneration.

In a still further embodiment, the present application provides a method of stimulating muscle tissue regeneration, the method comprising administering to a muscle an effective amount of a composition comprising or encoding secNAMPT or a part thereof comprising a cytokine finger (cif) motif or a derivative thereof comprising a cytokine finger (cif) motif, and a tissue specific delivery moiety, wherein the secNAMPT, part or derivative binds to satellite cells and stimulates satellite cell activation, myoblast proliferation and muscle regeneration and the absence of substantial fibrosis (scar formation).

Reference to NAMPT and CCR5 includes homologues and orthologs thereof from any animal including mammals, non-mammalian vertebrates, fish and birds.

In one embodiment, NAMPT or a part thereof comprising a cytokine finger (cif) motif or a derivative thereof comprising a cytokine finger (cif) motif, comprises the amino acid sequence set out in one of SEQ ID NO: 1 to 4 or an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical thereto.

In one embodiment, a NAMPT part or fragment thereof comprising a cytokine finger (cif) motif or a derivative thereof comprising a cytokine finger (cif) motif, comprises the amino acid sequence set out in one of SEQ ID NO. 1 or 2 or an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical thereto.

In one embodiment, a nucleic acid molecule encoding full length NAMPT or a functional part thereof comprises the polynucleotide sequence set out in one of SEQ ID NO: 8 or 9, or a polynucleotide sequence that has at least 70%, 75%, 80%, 85%, 86%, 87%, 88%, 89%, 90%, 91%, 92%, 93%, 94%, 95%, 96%, 97%, 98%, 99%, or 100% sequence identity thereto.

In one embodiment, a nucleic acid molecule encoding a NAMPT part or fragment thereof comprising a cytokine finger (cif) motif or a derivative thereof comprising a cytokine finger (cif) motif, comprises the polynucleotide sequence set out in one of SEQ ID NO: 6 or 7, or a polynucleotide sequence that has at least 70%, 75%, 80%, 85%, 86%, 87%, 88%, 89%, 90%, 91%, 92%, 93%, 94%, 95%, 96%, 97%, 98%, 99%, or 100% sequence identity thereto.

In one embodiment, NAMPT or a part thereof comprising a cytokine finger (cif) motif or a derivative thereof comprising a cytokine finger (cif) motif, having a small number of substituted or deleted residues while retaining the cif motif mediated ability to interact with stem cells or their more differentiated progeny, such as satellite cells. In one embodiment, NAMPT or a part thereof comprising a cytokine finger (cif) motif or a derivative thereof comprising a cytokine finger (cif) motif, comprises amino acid sequence having 1, 2, 3, 4, 5, 6, 7 or 8 conservative or non-conservative amino acid substitution, deletion or addition to the above sequences and retain CCR5 or tissue stem cell interacting activity. In particular, portions of between 1 and 5 contiguous amino acids may be deleted from NAMPTcif (SEQ ID NO: 1, 2, 5) and provide a functional derivative able to induce CCR5 signalling through the Mapk/Erk signalling cascade.

In some embodiments, NAMPT parts or fragments comprise a C-terminal portion of full length secNAMPT that binds CCR5 and do not comprise portions that are capable of enzymatic activity.

In some embodiments NAMPT parts or fragments comprise a C-terminal portion of full-length secNAMPT having a chemokine-like α-helix and β-sheets, that binds CCR5.

In one embodiment, NAMPT peptides that do not comprise the cif motif may be administered intracellularly to initiate activation of quiescent muscle cells.

In one embodiment, the NAMPT peptide comprises the nicotinamide ribonucleotide binding sites (amino acids 219, 384, 392, 311-313, 353-354) or diphosphate binding sites 196, 247, and 311).

In one embodiment, CCR5 agonists and NAMPT or parts or fragments thereof comprise tissue delivery or retention enhancing moieties such as one or more ECM and/or other tissue specific binding moieties. Without being bound by particular modes of action, NAMPT and NAMPT derivatives may be particularly selective in binding to a target tissue stem cell that bear their receptor and not to other cells bearing the receptor, thereby improving the selectivity of the method.

In one embodiment, CCR5 agonists and NAMPT or parts or fragments thereof comprise one or more signalling enhancing moieties such as a syndecan binding moiety.

In one embodiment an extracellular matrix (ECM) binding moiety known in the art is included. Illustrative ECM binding peptides are described in US publication no. 2014/0011978 and US publication no. 20140010832. Standard methods are used to conjugate agents or peptides to moieties such as ECM binding moieties with or without linkers.

In another embodiment, a syndecan binding moiety is included to provide tonic or enhanced CCR5 signalling via syndecans.

In one embodiment, NAMPT parts or fragments are administered as monomers, dimers or in multimeric form. In one embodiment, dimers display increased receptor or tissue stem cell binding relative to the monomeric form.

In one embodiment, the composition is a cellular composition comprising a cell that expresses the CCR5 interacting agent, specifically NAMPT or a functional derivative as described or illustrated herein.

In one embodiment, the present application provides method of stimulating muscle tissue regeneration, the method comprising administering to a muscle an effective amount of a composition comprising a cell comprising or encoding a CCR5 interacting agent, and optionally a component that enhances delivery to or retention in the muscle, wherein the CCR5 interacting agent binds to satellite cells and stimulates myoblast proliferation and muscle regeneration.

In one embodiment, the cell expresses endogenous NAMPT and/or an introduced NAMPT or a functional derivative thereof.

In one embodiment, the cell is a macrophage. In one embodiment the macrophage is isolated from tissue. In one embodiment, the macrophage is induced from stem cells such as bone marrow precursors or iPSC. In one embodiment, the macrophage or macrophage precursor (a monocyte) is isolated from a supply tissue such as, but not limited to blood, lymph, bone marrow) and then subjected to in vitro cell or tissue culture to induce the desired tissue niche directed phenotype. In one embodiment, the cell composition is cryopreserved and/or contains a delivery agent.

As known in the art, macrophages may be generated in vitro from stem cells by various means. Macrophages generated from stem cells, such as BMSC, in the presence of IFNg or LPS are generally considered as “inflammatory” macrophages referred to as “M1 macrophages.” Those generated in the presence of IL-4 or IL-10 have what is called a “pro-resolution” activity and are referred to as “M2” macrophages.

In one embodiment, the subject macrophage expresses M2 macrophage markers.

In one embodiment, the macrophage cell expresses one or two or three or four or five of mmp9, arg2, mmp13a, L-plastin and cd163.

In one other embodiment, the macrophage subset expresses prox1a and pou2f3.

In one embodiment, the macrophage is a Cluster macrophage as described and defined herein, such as a Cluster 1, Cluster, 2, Cluster 3, Cluster 4, Cluster 5, Cluster 6, Cluster 7, or Cluster 8 cell type. In one embodiment, the Cluster macrophages have the herein described and define differentiating features before culture expansion. In one embodiment cells are administered in an amount determined by the attending practitioner, such as, for example about 1×10⁷ or between about 1×10⁷ and 2×10⁸ cells. Cells administered are predominantly of one or more Cluster types as defined herein. The Examples and supplementary tables set out the differentially expressed genes/markers that may be used for selecting, detecting, quantifying, functionally characterising, and differentiating between predominant macrophage/cell cluster types. Markers may be surface markers or internal markers or both, as known in the art. Thus markers may be selected based on the level/extent of differential expression to facilitate selection/detection which genes that are most upregulated or down regulated taking preference. Alternatively, markers and therefore cells may be selected based on the functional activity or phenotype of the cluster cell type wherein the detection of the marker indicates a desirable/undesirable functional feature of the cell. One, several or multiple markers may be employed depending upon the rationale for selection, ie, cell enrichment, cell selection for administration, cell tracing, cell targeting etc.

In one embodiment, the composition further comprises a stem cell and/or a macrophage cell.

In one embodiment, the stem cell is a satellite cell. In another embodiment the stem cell is a unipotent or multipotent stem cell.

In one embodiment of the method, the CCR5 interacting agent in the composition is NAMPT or a part thereof comprising a cytokine finger (cif) motif or a functional derivative thereof, or a pharmaceutically acceptable salt, hydrate, homolog, ortholog, tautomer, sterioisomer, pro-drug thereof.

Pro-drugs refer to agents that can be converted via some chemical or physiological process (e.g., enzymatic processes and metabolic hydrolysis) to a CCR5 agonist. Thus, the term “prodrug” also refers to a precursor of a biologically active compound that is pharmaceutically acceptable. A prodrug may be inactive when administered to a subject but is converted in vivo to an active compound. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively.

In one embodiment, the CCR5 interacting agent or NAMPT or a part thereof comprising a cytokine finger (cif) motif or a derivative thereof, comprises one or more moieties such as a linker, stability enhancing, signalling enhancing, delivery enhancing or label moiety.

In one embodiment, the CCR5 interacting agent is in monomeric, dimeric or multimeric form.

In one embodiment, the CCR5 interacting agent or muscle stem cell interacting agent is NAMPTcif in monomeric, dimeric or multimeric form and also comprising a tissue directed or ECM binding domain and/or a signalling enhancing (eg a syndecan binding) domain.

In one embodiment, the composition encoding the CCR5 interacting agent comprises a nucleic acid molecule that expresses the CCR5 interacting agent. The nucleic acid molecule may be an RNA or DNA or RNA:DNA or a chemically modified form thereof. For example, the nucleic acid may be in the form of a viral or non-viral vector.

In another embodiment, the CCR5 interacting agent is a small molecule. The present application provides screening assays for screening small molecule CCR5 agonists able to induce tissue stem cell activation. A “small molecule” is defined herein to have a molecular weight below about 1000 Daltons, preferably below about 500 Daltons.

In another embodiment, the CCR5 interacting agent is an antibody or comprises a CCR5 binding part thereof.

In another embodiment, the CCR5 interacting agent comprises an antibody or antibody fragment that targets the agent to myeloid cells such as macrophages, or targets the agent to stem cells such as satellite cells.

The present application provides compositions comprising or encoding the CCR5 interacting agent as defined herein. Pharmaceutical and physiologically active compositions are provided. Cellular compositions are expressly provided.

In one embodiment, the cell is a macrophage. In one embodiment the macrophage is isolated from tissue. In one embodiment, the macrophage is induced from stem cells such as bone marrow precursors or iPSC. In one embodiment, the macrophage or macrophage precursor (a monocyte) is isolated from a supply tissue such as, but not limited to blood, lymph, bone marrow) and then subjected to in vitro cell or tissue culture to induce the desired tissue niche directed phenotype. In one embodiment, the cell composition is cryopreserved and/or contains a delivery agent.

As known in the art, macrophages may be generated in vitro from stem cells by various means. Macrophages generated from stem cells, such as BMSC, in the presence of IFNg or LPS are generally considered as “inflammatory” macrophages referred to as “M1 macrophages.” Those generated in the presence of IL-4 or IL-10 have what is called a “pro-resolution” activity and are referred to as “M2” macrophages.

In one embodiment, the subject macrophage expresses M2 macrophage markers.

In one embodiment, the macrophage cell expresses one or two or three or four or five of mmp9, arg2, mmp13a, L-plastin and cd163.

In one other embodiment, the macrophage subset expresses prox1a and pou2f3.

In one embodiment, the macrophage is a Cluster macrophage as described and defined herein, such as a Cluster 1, Cluster, 2, Cluster 3, Cluster 4, Cluster 5, Cluster 6, Cluster 7, or Cluster 8 cell type. In one embodiment, the Cluster macrophages have the herein described and define differentiating features before culture expansion. In one embodiment cells are administered in an amount determined by the attending practitioner, such as, for example about 1×10⁷ or between about 1×10⁷ and 2×10⁸ cells. Cells administered are predominantly of one or more Cluster types as defined herein. The Examples and supplementary tables set out the differentially expressed genes/markers that may be used for selecting, detecting, quantifying, functionally characterising, and differentiating between predominant macrophage/cell cluster types. Markers may be surface markers or internal markers or both, as known in the art. Thus markers may be selected based on the level/extent of differential expression to facilitate selection/detection which genes that are most upregulated or down regulated taking preference. Alternatively, markers and therefore cells may be selected based on the functional activity or phenotype of the cluster cell type wherein the detection of the marker indicates a desirable/undesirable functional feature of the cell. One, several or multiple markers may be employed depending upon the rationale for selection, ie, cell enrichment, cell selection for administration, cell tracing, cell targeting etc.

In one embodiment, the composition comprises or is administered together with a supporting material such as a hydrogel, glue, foam or retentive material, scaffold etc. Delicate structures are generally suitable for enabling more delicate tissue regeneration. As examples, materials can be used which are quite rapidly absorbed, such as certain fibrin, collagen, hydrogel and alginate formulations. Alternatively, slowly absorbable synthetics can be used, such as poly-4-hydroxybutarate. Silk fibers or even substantially smooth products derived from mammalian origin such as muscle extracellular matrix are also contemplated. Non-absorbable synthetics, such as polypropylene and polyethylene, provide support and reliability. In one embodiment the composition comprises a fibrin hydrogel. In another embodiment, RAFT-acrylamide based support surfaces are provided to enhance tissue regeneration and bioavailability of CCR5 interacting agent to the target site.

In one embodiment, the present application provides a NAMPT polypeptide fragment comprising a C-terminal portion of NAMPT comprising a cytokine finger (cif) motif, or a modified form or functional derivative thereof.

In one embodiment, the NAMPT polypeptide fragment comprises the peptide sequence set forth in SEQ ID NO: 1 or 2 or 5 (monomeric NAMPTcif) or a sequence having at least 80% identity to SEQ ID NO: 1 or 2 or 5.

In one embodiment, a nucleic acid molecule is provided encoding a NAMPT polypeptide fragment comprises the peptide sequence set forth in SEQ ID NO: 1 or 2 or (monomeric NAMPTcif) or a sequence having at least 80% identity to SEQ ID NO: 1 or 2 or 5. Illustrative nucleic acid sequences are set forth in SEQ IN NO: 6 to 9 and Table 4. In one embodiment, the nucleic acid molecule comprises least 80% identity to SEQ ID NO: 6 or 7 (mouse and human NAMPTcif respectively).

Reference herein to at least 80% sequence identity includes explicitly molecules having at least 81%, 85%, 86%, 87%, 88%, 89%, 90%, 91%, 92%, 93%, 94%, 95%, 96%, 97%, 98%, 99%, or 100% sequence identity thereto.

In one embodiment, the fragment or part of NAMPT comprises a number of NAMPT amino acids which is less than 10%, 15%, 20%, 25%, 30%, 35%, 40% 50% 60% 70% 80% 85%, 86%, 87%, 88%, 89%, 90%, 91%, 92%, 93%, 94%, 95%, 96%, 97%, 98%, 99%, or 100% of the full length NAMPT set forth in SEQ ID NO: 3.

In one embodiment, the NAMPT polypeptide fragment further comprising a linker, stability or signalling enhancing moiety, delivery or retention enhancing moiety or label moiety.

In one embodiment, the NAMPT fragment is in monomeric, dimeric or multimeric form. Protein re-folding protocols are known in the art.

In one embodiment is provided a composition comprising the NAMPT polypeptide parts or fragment as described herein and any one or two or three or four of: (i) a satellite cell or precursor therefore or progeny thereof (ii) a macrophage or a precursor therefore or progeny thereof (iii) a scaffold (semi-solid or solid support) or retentive material (iv) a tissue delivery enhancing or cell retention moiety.

In one embodiment the scaffold or retentive material is a hydrogel, such as a fibrin or acrylamide hydrogel. In one embodiment, the tissue delivery enhancing or cell retention moiety is an ECM-binding moiety.

In one embodiment, the application enables a pharmaceutical or physiologically active regenerative composition comprising one or two or three or four or five of
(i) comprising or encoding a CCR5 interacting agent as defined herein,
(ii) a satellite cell or precursor therefore or progeny thereof
(iii) a macrophage or a precursor therefore or progeny thereof
(iv) a scaffold or retentive material
(v) a tissue delivery enhancing component.

In a particular embodiment, as described elsewhere herein, the CCR5 interacting agent in the form of full length NAMPT or the cytokine interacting fragment (cif) thereof in monomeric or dimeric form is modified to make the agent suitable for attachment to a biological carrier or to the extracellular matrix. In addition, the agent is modified to enhance signalling through the CCR5 receptor by addition of moieties that bind co-receptors such as heparin sulphate proteoglycans (such as syndecans).

While the CCR5 interacting agent including proposed modification have been illustrated in the examples using NAMPT, the experiments described herein show that CCR5 agonists CCL4 and CCL8 also operate in skeletal muscle satellite cells to stimulate muscle myoblast proliferation. Thus CCL4 and CCL8 are further illustrative CCR5 agents and full length, parts and fragments (comprising a cif motif), and derivatives thereof are proposed for use in the present methods and for use in the manufacture of regenerative compositions as described herein. In a particular embodiment, as described elsewhere herein, the CCR5 interacting agent in the form of full length CCL4 or CCL8 or the cytokine interacting fragment (cif) thereof in monomeric or dimeric form is modified to make the agent suitable for attachment to a biological carrier or to the extracellular matrix. In addition, the agent is modified to enhance signalling through the CCR5 receptor by addition of moieties that bind co-receptors such as heparin sulphate proteoglycans (such as syndecans).

In one embodiment, the compositions described herein are for use, or for use in manufacturing compositions for use, in stimulating muscle regeneration in vitro, ex vivo or in vitro.

In one embodiment, the compositions described herein are for use or when used in artificial muscle production (such as fish, bird or other non-human animal muscle for direct or indirect consumption). For example, NAMPT supplementation to growth media enables scalability and more efficient muscle proliferation.

In one embodiment, the compositions described herein are for use or when used in stem cell therapy. Thus, the compositions support expansion in vitro and/or are included in a transplant (or as a pre-treatment) to promote in vivo expansion and tissue integration.

In one embodiment, the present application provides a method of stimulating tissue regeneration, the method comprising administering to an isolated or tissue-resident tissue stem cell or a precursor thereof an effective amount of a composition comprising or encoding a CCR5 interacting agent, and optionally a component that enhances delivery to the tissue, wherein the CCR5 interacting agent binds to tissue stem cells or their precursors and stimulates (activates) quiescent tissue stem cell proliferation and tissue regeneration. In one embodiment, the CCR5 interacting agent comprises a component or moiety that enhances delivery to the target tissue.

In one embodiment, the compositions described herein are for use or when used, or for use in manufacturing compositions for use, in treating a muscular, neuromuscular, or musculoskeletal deficiency, disorder or injury. Muscular, neuromuscular, or musculoskeletal deficiencies, disorders or injuries are known in the art. Deficiencies and disorders are found, for example, and without limitation in sarcopenia, cachexia and the muscular dystrophies, muscle atrophy, muscle pseudo hypertrophy or muscle dystrophy conditions and myopathies. All appropriate formats such as Swiss-style use, method of treatment and/or EPC 2000 style claims are encompassed.

### BRIEF DESCRIPTION OF THE FIGURES

The patent or application file contains at least one drawing executed in colour. Copies of this patent or patent application publication with colour drawing(s) will be provided by the Patent Office upon request and payment of the necessary fee.

The accompanying drawings, which are incorporated into and form a part of the specification, illustrate several embodiments of the present invention and, together with the description, serve to explain the principles of the invention.

FIG. 1. A subset of injury-responsive macrophages dwell at the wound site for the duration of repair. a-c, Uninjured muscle (Tg(actc1b:GFP), magenta) is patrolled by macrophages (MΦs) (Tg(mpeg1:GAL4FF/UAS:NfsB-mCherry), yellow) at 4 dpf (a). Upon injury, MΦs are activated and rapidly migrate into the wound site (b-b″, arrowheads) (n=3). (c) The migratory paths of injury-responding MΦs are graphed, where (0,0) is set to the centre of the wound. d-f, In toto imaging identifies two MΦ subsets occupying the wound at distinct temporal phases (f), an early injury-responsive transient (d) and late injury-located dwelling subset (e) (arrowheads) (n=8). g-i, Morphological characterisation of these two subsets revealed transient MΦs are more stellate (g) with lower sphericity (i), while dwelling MΦs are more rounded (h) with higher sphericity (i) (n=69 MΦs per group). Mean±S.D. Significance (****P<0.0001) in unpaired t-test. j-m, Tracking of wound located MΦs reveals that dwelling MΦs are derived from transient MΦs. (j) Photoconversion strategy schematic. At 1dpi, injury-located MΦs in Tg(mpeg1:Gal4FF/UAS:Kaede) larvae were photoconverted (k-k″, pre conversion; magenta (arrowheads), post conversion; yellow), reassessed at 2 dpi (l-l″) and quantified (m, n=20). Not significant (ns) in unpaired t-test. n-n′, Retrospective tracking of injury-present dwelling MΦs at 24 hpi identified their injury-proximate origin. (n) Dwelling MΦ track projections for the duration of imaging are superimposed on whole larvae. (n′) The location of dwelling MΦs (yellow) at different time points following imaging demonstrates their origin and sequential migration into the injury site (blue) (n=4).

FIG. 2. Dwelling macrophages induce muscle stem cell proliferation in vivo. a, Dosing strategy schematic for addition of the pro-drug metronidazole (Mtz) to ablate either all MΦs, or specifically dwelling MΦs in Tg(mpeg1:GAL4FF/UAS.NfsB-mCherry) larvae. b-h, Both these ablation strategies result in a significant regeneration deficit as observed by birefringence imaging (b-g′) and quantification (h, n=24). i-l, Following laser ablation injury (dotted line) transient MΦs migrate into the wound site (Tg(mpeg1:GAL4FF/UAS:NfsB-mCherry), yellow) (i-k). Simultaneously, activated pax3a+ myogenic stem cells (TgBAC(pax3a.GFP), cyan) from the injured and adjacent myotomes travel to line the edge of the injury site (i-k, arrowheads). Once MΦs transition to a dwelling subtype they initiate interactions with wound edge lining pax3a+ cells (1, orthogonal views) (n=5). [symbol] m. Length of uninterrupted macrophage-stem cell interactions for transient and dwelling MΦs quantified (extracted from n=5 injuries). Mean±S.D. Unpaired t-test. n, High-resolution AiryScan microscopy of these interactions reveal dwelling MΦs maintain prolonged intimate associations with pax3a+ muscle stem cells (arrowheads), following which the associated stem cell undergoes cell division (n=10). o-q, EdU incorporation assessment of stem cell proliferation following dwelling MΦ ablation o) reveals a requirement for this MΦ subset to maintain muscle stem cell divisions in the wound site. All EdU⁺ proliferating cells in the injury zone were co-labelled with pax3a and as such, myogenic stem/progenitor cells are the only proliferating cells present in the regenerate at this point in the repair process. The homeostatic level of myotome cell proliferation external to the injury zone provides an internal specificity control for these analyses, with no significant difference in cell proliferation in the presence or absence of dwelling MΦs being observed outside the wound. Quantification (q). Mean±S.D. Significance (****P<0.0001) in two-way ANOVA with Tukey's multiple comparison test.

FIG. 3. Single-cell RNA-seq identifies a unique mnp9 positive dwelling macrophage subset. a, Schematic of injury-responsive macrophage scRNA-seq workflow. b, UMAP scatter plot revealing spontaneous cell clusters. Following appropriate filtering, 1309 cells were analysed. c, In this graph, the injury time point of isolated MΦs is overlaid on the UMAP scatter plot and illustrates the correspondence between unsupervised clustering and the described transient and dwelling MΦ subtypes. d, Uninjured MΦs cluster together (Cluster 3). Macrophages isolated from a ‘transient’ time-point (1dpi) also predominantly cluster together (Cluster 1). The remaining 6 clusters (0, 2, 4, 5, 6, 7) are mainly composed of macrophages isolated from ‘dwelling’ time points (2-3 dpi). e, The macrophage identity of sorted cells was validated by their collective expression of the pan-leukocyte marker lcp1 (L-plastin) and pan-macrophage marker cd163 (cd63). Known pro-regenerative MΦ markers arg2, mmp9 and mmp13a are concentrated in cluster 2 present MΦs. The percentages of cells in Cluster 2 expressing these markers are shown. In addition, this cluster also differentially expresses nampta. Gene expression levels visualised in feature plots are log-normalised gene read counts using a scaling factor of 10,000. f, Violin plots show the expression pattern of mmp9 and nampta based on cluster identity and isolation time point. The percentage of cells expressing the markers at 2 dpi is documented. g-k. Lineage analysis was performed using partition-based graph abstraction (PAGA). Ball-and-stick representation of PAGA connectivity; pie charts represent clusters (size reflective of cluster cell size) showing their macrophage composition based on isolation time; edge thickness indicates the statistical measure of connectivity among clusters (g). Cells were re-embedded using PAGA-initialised force-directed layout (FDL), where cells were grouped according to their isolation time point identity (h) Seurat cluster identity (i) and pseudotime inferences (j). (k) PAGA path graph visualises gene expression changes leading to the definition of a mmp9-dwelling macrophage subset along PAGA cluster's path 3-0-4-2 and a prox1a⁺/pou2f3⁺-dwelling macrophage subset along PAGA cluster's path 3-5-1-6. Numerical scale (right) expresses normalised gene expression from Seurat, while clusters (lower) indicates subsets along the PAGA path with length being proportional to cell numbers in each cluster. l. The spatiotemporal expression of mmp9 RNA was assayed by in situ hybridisation following needle stab injury. mmp9 expression is specifically up-regulated in the wound site from 2 dpi onwards (n>15). m-n, mmp9 expression (TgBAC(mmp9:EGFP), magenta) in the wound site was spatially resolved and identified to be present in a subset (white arrowheads) of dwelling MΦs (Tg(mpeg1:GAL4FF/UAS:NfsB-mCherry), yellow). Quantification (n). Mean±S.D. Unpaired t-test. (o) These mmp9-expressing MΦs (magenta) were associated with wound-present pax3a⁺ stem cells (cyan, wound site: white dotted line, n=15). p-r, Temporally controlled metronidazole (Mtz)-based ablation of mmp9-expressing dwelling MΦs in TgBAC(mmp9:EGFP-NTR) larvae leads to a significant skeletal muscle regeneration deficit (p). Quantification (q). Assaying EdU incorporation in mmp9⁺ MΦ-ablated larvae revealed a significant reduction in wound site located muscle stem cell proliferation (r). Representative images are presented in FIG. 10g. Mean±S.D. Two-way ANOVA with Tukey's multiple comparison test (q,r).

FIG. 4. Macrophage secreted Nampt binding to Ccr5 induces stem cell proliferation. a, Within the injury site (white dotted line) Nampt (magenta) is expressed by macrophages (yellow, red arrowheads highlight Nampt/mpeg1⁺ MΦs) associated with muscle stem cells (cyan), as well as being present in the extracellular space (n=15). b-i, A novel tissue specific loss-of-function mutagenesis strategy was utilised to independently assess Nampta's specific role in injury-responsive macrophages (schematic, b) and Ccr5's specific role in muscle stem/progenitor cells (schematic, c). Mpeg⁺ cell-specific Nampta and Pax7b⁺ cell-specific Ccr5 knockout larvae, both displayed marked regeneration deficits (imaged (d) and quantified (e)). In both cases, this was due to an inability to maintain the required proliferative response in wound-resident muscle stem cells (imaged (f, h) and quantified (g, i)). j, NAMPT supplementation specifically enhanced wound-resident muscle stem cell proliferation following larval zebrafish needle stab muscle injury. NAMPT supplementation rescues the proliferation deficit generated by ablating macrophages (5 mM Mtz added at 4 dpf/0 dpi). However, NAMPT failed to rescue the proliferation defect generated by administering the Ccr5/Ccr2 dual-antagonist cenicriviroc (CVC). While the canonical Ccr5 ligand CCL4 functioned to enhance proliferation it failed to demonstrate discrimination between injury region and sites external to the damage. Probability values and significance of experimental conditions verses untreated control recorded on top of each violin plots. Representative images are presented in (FIG. 14d. k, PAX7⁺ satellite cells in mouse primary myoblast monocultures display enhanced proliferation upon CCR5 receptor signalling, mediated by either exogenous NAMPT or CCL4 supplementation. Co-culturing myoblasts with macrophages stimulated satellite cell proliferation. This pro-proliferative response is abolished by administration of the CCR5-specific inhibitor maraviroc (MVC), while the CCR2-specific inhibitor PF-4136309 (PF4) had no negative effect, reaffirming that macrophage's pro-proliferative function on stem cells is mediated by CCR5 signalling. In addition, co-culturing myoblasts with 3T3 cells that do not secrete NAMPT does not stimulate satellite cell proliferation, suggesting NAMPT as the macrophage driving pro-proliferative cue driving stem cell proliferation. e, g, i, j, k, Mean±S.D. Two-way ANOVA with Tukey's multiple comparison test. l-o, Local delivery of NAMPT promotes muscle regeneration in an adult mouse muscle injury model (schematic, 1). (m) Volumetric muscle defects were created and directly treated with NAMPT delivered via a fibrin hydrogel. Masson's trichrome stained representative tissue sections of murine rectus femoris (RF) muscle (10 days post treatment) through the middle of the defect, demonstrate NAMPT delivery significantly increased the regenerated muscle area (dark red, quantification, n) while simultaneously showing a significant reduction in fibrotic tissue (purple/blue, white dashed line demarcates the separation between fibrotic and healthy muscle fibres, while the fascia surrounding the muscle is stained in blue (quantification, o)). n,o, Means±SEM. One-way ANOVA with Dunnett's post hoc test for multiple comparisons (n=5 mice per group). p-t, Satellite cells demonstrated enhanced proliferation upon exogenous NAMPT supplementation. Mouse muscle injuries were treated with NAMPT (0.5 μg) delivered in fibrin or fibrin only control. (p-q) The total number of satellite cells (PAX7⁺) (p) and the number of satellite cells in the proliferation phase (PAX7⁺/Ki67⁺) (q) was quantified by flow cytometry in tissues harvested 4 days post treatment. The graphs show the fractions of satellite cells per 10,000 cells in the harvested tissue (n=6 mice for the fibrin group, n=5 mice for NAMPT-treated group). The gating strategy is shown in FIG. 10e. (r) Representative muscle regenerate cryosections stained for PAX7 (satellite cells, yellow), wheat germ agglutinin (WGA, magenta), and nuclei (DAPI, blue) for tissues harvested 6 days post treatment. (s-t) Centrally nucleated muscle fibres were quantified at 6 days post treatment (n=6 mice per group)(s). Representative histology tissue sections with Haematoxylin and Eosin (t). p,q,s, Mean±S.E.M. Two-tailed Student's t-test.

Dwelling macrophages establish a transient niche following muscle injury that is indispensable for the regenerative process. a-c′″, Maximum intensity projection images of the same individual fish transgenic for Tg(actc1:BFP), labelling differentiated muscle fibres (magenta, a′, b′, c′), TgBAC(pax3a:GFP), labelling par3a⁺ myogenic cells (cyan, a″, b″, c″) and Tg(mpeg1:GAL4FF/UAS:NfsB-mCherry) labelling macrophages (yellow, a′″, b′″, c′″) following needle stab muscle injury. d, By 2 dpi 51.6% of the original injury-responsive MΦs remain in the wound site, and these dwelling MΦs go on to establish a transient injury niche that is pro-myogenic (n=20). e-i, Establishing nitroreductase-mediated macrophage ablation parameters. (e-i) The transgenic line, Tg(mpeg1:GAL4FF/UAS:NfsB-mCherry) expresses the enzyme nitroreductase specifically within macrophages, enabling the temporally-controlled genetic ablation of macrophages by addition of the pro-drug metronidazole (Mtz). The optimum Mtz concentration was established by treating larvae with 5 and 10 mM Mtz for 24 h (starting at 4 dpf) and visualising (e) and quantifying (f) trunk-present MΦs. 77% MΦ ablation was achieved at 5 dpf by 5 mM Mtz treatment (f, n=10). Although uninjured larvae were able to tolerate 10 mM Mtz, needle stab injury carried out at this concentration resulted in significant lethality. Therefore, all further experiments used 5 mM Mtz for MΦ ablations. (g-g′) Titrating the Mtz dosing time to specifically ablate MΦ subsets of interest. (g) Temporal response to 5 mM Mtz was visualised by time-lapse imaging the head and tail region of larval zebrafish immediately following Mtz addition (4 dpf). MΦ ablation initiates at 3 h post Mtz treatment. Frames extracted from Supplementary Video. 5 (n=8). (g′) Addition of Mtz at the point of injury ablated all MΦs (yellow, shown superimposed on brightfield images of the zebrafish trunk), while addition of Mtz at 1.75 dpi (5 dpf) selectively ablated dwelling MΦs (n=10). (h-i) Ablation of macrophages did not alter the neutrophil response to wounding at 2 dpi (imaged (h) and quantified (i), n=12). j, The temporal sequence of neutrophil migration in response to skeletal muscle injury, was assayed and quantified (k, n=14 at 5-6 hpi, n=13 at 1 dpi and n=12 at 2 dpi). l-o, As a control experiment for nitroreductase mediated cell ablation, neutrophils were selectively ablated using the transgenic line Tg(mpx:KALTA4/UAS:NfsB-mCherry). Ablation efficiency following 24 h of 5 mM Mtz treatment starting at 4 dpf was visualised (1) and quantified at 5 dpf (m, n=10). (n) Following needle stab injury, larvae were soaked in 5 mM and 10 mM Mtz (4 dpf/0 dpi) to ablate all neutrophils and 5 mM Mtz at 5 dpf (1.75 dpi) to specifically ablate late injury-present neutrophils (Mtz treatment was maintained until experimental end point at 7 dpf/3 dpi with daily Mtz drug refreshment) and regeneration monitored by imaging for birefringence (n) and quantified (o, n=12). Neutrophil ablated larvae showed no regeneration defects at either Mtz dose or timing, highlighting a specific requirement for macrophages in stimulating regeneration and precluding the possibility that any regeneration defect was metronidazole-related. d, f, k, m, Mean±S.D. One-way ANOVA with Dunnett's post hoc test for multiple comparison. i, Mean±S.D. Unpaired t-test. o, Mean±S.D. Two-way ANOVA with Tukey's multiple comparison test.

FIG. 6. Macrophage ablation does not disrupt tissue debris clearance following muscle injury. a-f, At 3 dpf macrophages within the Tg(mpeg1:GAL4FF/UAS:NfsB-mCherry) (macrophages expressing nitroreductase) transgenic line were ablated in larval zebrafish by the addition of 5 mM metronidazole (Mtz). Macrophage-ablated larvae were then subject to a needle stab skeletal muscle injury at 4 dpf and the wound site assayed for clearance of damaged muscle fibres by a number of criteria. (a) phalloidin staining, marking remnant muscle fibres. No difference is evident between control and macrophage-ablated larvae. n=3 independent injuries per time point presented, n=12 injuries per time point imaged. (b-c) AnnexinV marking the apoptotic/phagocytic signalling process was visualised in vivo using the transgenic line Tg(ubi:secAnnexinV-mVenus)(magenta, n=3 independent injuries per time point presented)(b) and quantified (c). (d-e) Acidic organelles marked by LysoTracker (magenta) were imaged (d) and quantified (e). (c, e) Mean±S.D. Two-way ANOVA with Tukey's multiple comparison test (c) or unpaired i-test (e). No difference is evident between control and macrophage-ablated larvae. (f) Haematoxylin and Eosin-stained cross section through needle stab injured myotomes demonstrates cleared wound site (black arrowheads) at 3 days following wounding in macrophage-ablated larvae (n=3 larvae per group).

FIG. 7. Dwelling macrophage-muscle stem cell associations precede muscle stem cell proliferation. a-c, The pax3a⁺-myogenic cells that dwelling macrophages interact with also express the muscle stem cell markers met (a-b) and pax7b (c). (a) Following needle stab skeletal muscle injury (white dotted line) in larvae transgenic for, TgBAC(pax3a:GFP) (cyan) and Tg(met:mCherry-2A-KALTA4/UAS:NfsB-mCherry) (magenta), wound-present muscle stem cells are pax3a⁺/met⁺. These cells undergo both symmetric expansion (white open arrowheads) and asymmetric divisions (closed yellow arrowheads), giving rise to two pax3a⁺/met⁺ daughter cells or a pax3a⁺/met⁺ and a pax3a⁺/met⁻ daughter cell, respectively. The broad myogenic marker pax3a⁺ visualises each of these events (n=20). (b) Time lapse imaging was used to visualise muscle stem cells (TgBAC(pax3a:GFP)/Tg(met:mCherry-2A-KALTA4/UAS:NfsB-mCherry), cyan/yellow double positive cells) and dwelling macrophage (Tg(mpeg1:GAL4FF/UAS:NfsB-mCherry), yellow) interactions following a laser ablation skeletal muscle injury (white dotted line). Dwelling macrophages (white closed arrowhead) interact with pax3a⁺/met⁺ muscle stem cells (magenta open arrowhead), following which the muscle stem cell undergoes symmetric division to generate two pax3a⁺/met⁺ progenitors. Frames extracted from Supplementary Video. 9 (n=6). (c) Wound site (white dotted line)-present dwelling macrophages (Tg(mpeg1:GFP), yellow) interact with muscle stem cells that also express the marker pax7b (cyan, Tg(pax7b:GAL4FF/UAS:NfsB-mCherry)). d-f, Muscle stem cells migrate into the injury site independent of macrophage-derived signals. As macrophages (Tg(mpeg1:GAL4FF/UAS:NfsB-mCherry), yellow) and muscle stem cells (TgBAC(pax3a:GFP), cyan) migrate and populate the injury site simultaneously, the dependence of one on the other was assessed. Both control larvae and macrophage ablated larvae (5 mM Mtz added at point of injury (4 dpf) to ablate all macrophages) displayed pax3a⁺ stem cells in the injury site (white dotted line) at 2 dpi (white arrowheads) following needle stab skeletal muscle injury (d) and muscle stem cells lining the edge of the injury site (yellow asterisks) following laser ablation muscle injury at 14 hpi (e). Quantification of laser injury-responsive macrophages (f). Mean±S.D. Not significant in unpaired t-test. In the control setting (top panel d, e) these cells were associated with macrophages. At 3 dpi following needle stab injury, control larvae displayed regenerated cyan fluorescence-persistent muscle fibres (red arrowheads) that arose from pax3a⁺ muscle stem cells present in the wound (d), a hallmark of a healing muscle injury. In contrast, although the wound site was still occupied by pax3a⁺ stem cells at 3 dpi in the macrophage ablated larvae, there were no nascent pax3a⁺ stem cell-derived muscle fibres present (d), highlighting that macrophage presence in the wound site is not prerequisite for pax3a⁺ myogenic cell migration, however, it is required for those migrated cells to enter the myogenic replenishment program (n=12). g, Dwelling macrophages interact with wound-responsive muscle stem cells. Examples of 3 independent laser ablation muscle injury sites showing dwelling macrophage-stem cell associations followed by stem cell divisions (arrowheads). Imaging carried out in transgenic zebrafish larvae, Tg(mpeg1:GAL4FF/UAS:NfsB-mCherry), labelling macrophages (yellow) and TgBAC(pax3a:GFP) labelling pax3a⁺ cells (cyan). Frames extracted from Supplementary Video. 7. h, Following muscle stem cell division the associated dwelling macrophage, and generated daughter cells, migrate away from each other. Dwelling macrophages interact with muscle stem cells located within the injury zone (white dotted line). Following muscle stem cell division (closed white, magenta, yellow and grey arrowheads highlight 4 independent stem cells) the daughter cells (open white, magenta, yellow and grey arrowheads highlighting the daughter cells that arose from the stem cells indicated by closed arrowheads of the same colour) movements within the wound site are highlighted such that their relation to the dwelling macrophage can be visualised. Cell movements are tracked until dwelling macrophages localise at the vertical myosepta. Frames extracted from Supplementary Video. 10. Representative of n=9 stem cell divisions as assayed in n=5 long-term time lapses.

FIG. 8. Correlative light and electron microscopy (CLEM) analyses of the transient macrophage-stem cell niche reveals that macrophages and stem cells maintain direct hetero-cellular surface appositions in x-y-z planes. a, Confocal microscopy of the wound site at 25 hours post-laser ablation muscle injury in the compound transgenic zebrafish line, Tg(mpeg1:GAL4FF/UAS:NfsB-mCherry); TgBAC(pax3a:GFP), labelling macrophages (yellow) and pax3a⁺ muscle stem/progenitors (cyan). b, Large STEM tile set of the same trunk region of the identical larvae illustrated in (a) generated after Epon-embedding and sectioning. This data set was used to correlate and identify the highlighted macrophage-stem cell interaction of interest (white asterisk (a,b)). Dotted square marks the area that was further examined by transmission electron microscopy (in c,d). c, Region of interest encompassing a macrophage and stem cell which maintain a close interaction examined through z-depth. The cells of interest and interaction area are segmented (macrophage cytoplasm: yellow, macrophage nucleus: orange, stem cell cytoplasm: cyan, stem cell nucleus: blue, cell-cell interaction surface: magenta). d, high resolution images of two planes through the z-depth (plane shown correlated with red dotted lines to the segmentation images) further demonstrate the close association between the two cells (black scale bar: 5 μm).

FIG. 9. Prox1a⁺/Pou2f3⁺ macrophages (cluster 6) identified by scRNA-seq are a dwelling macrophage subset independent from Mmp9⁺ macrophages (cluster 2). a, Cells assayed by scRNA-seq express antigen processing and presentation genes, confirmatory of their macrophage character. Feature plots highlighting antigen presenting genes expressed by the majority of cells (cd83, cd81a and cd40) as well as differentially expressed by individual subsets are presented. The percentage of cells expressing a gene of interest is also recorded (maroon, information extracted from Supplementary Table. 1,3). b-g, Violin plot (with percentage of cells in cluster 6 expressing marker recorded as percentage (b, e), and antibody staining for subset 6-specific markers Pou2f3 (c) and Prox1a (f) following needle stab muscle injury (white dotted line) identified a specific, late injury-dwelling, macrophage population (Supplementary Table. 3, worksheet. 7). Antibody staining against mCherry was used to identify all mpeg1⁺ macrophages in the Tg(mpeg1:GAL4FF/UAS:Nfsb-mCherry) (yellow) line. Following injury, at 1 dpi, almost no Pou2f3⁺/mpeg⁺ (c) or Prox1a⁺/mpeg⁺ (f) macrophages are present in the wound site, their numbers start to increase slightly at 2 dpi with the highest percentage being present at 3 dpi, highlighting this subset as a late injury-dwelling macrophage subset. Quantification (d, g). Mean±S.D. Unpaired t-test. (d) One-way ANOVA with Dunnett's post hoc test for multiple comparison test. (g). h-i, Cluster 2 macrophages display a metabolic shift towards glycolysis at the gene expression level. KEGG pathway analysis of cluster 2 differentially expressed genes (Supplementary Table. 3) identified genes associated with glycolysis (term: dre00010) and oxidative phosphorylation (term: dre00190) pathways. Genes associated with glycolysis were up-regulated (h), while the majority of genes associated with oxidative phosphorylation were down-regulated (i), highlighting a metabolic shift towards glycolysis in cluster 21mmp9⁺-dwelling macrophages.

FIG. 9. a. NAMPT contains a “cytokine finger” (cif) conserved in other cytokines. b. NAMPTcif inhibits the binding of NAMPT to CCR5. c. Satellite cell stimulation with NAMPTcif promotes proliferation in a dose-dependent manner compared to no stimulation control baseline (No stim.). d. Derivatives of NAMPT, with or without ECM and syndecan-binding tags are tested in vivo and in vitro.

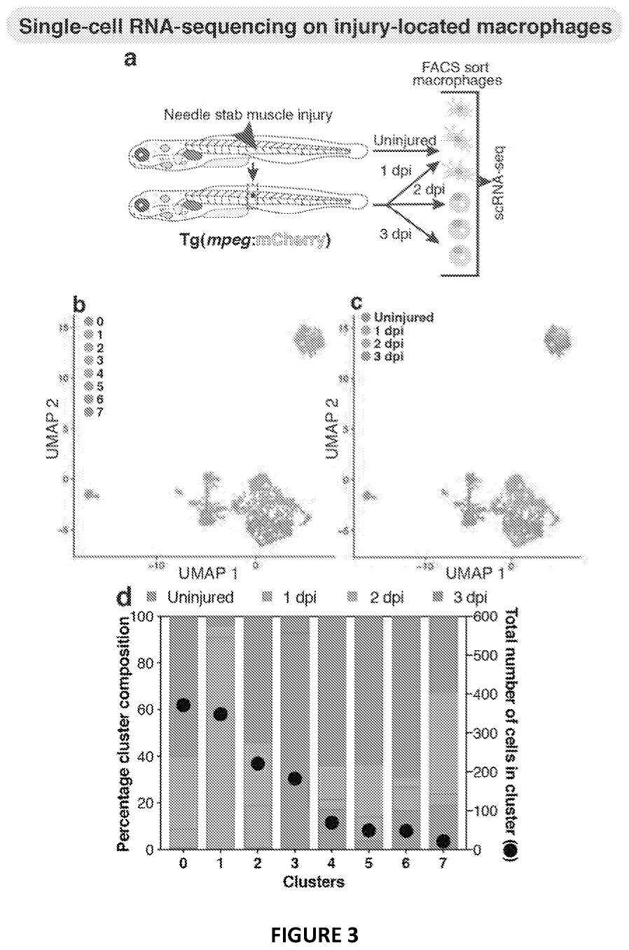

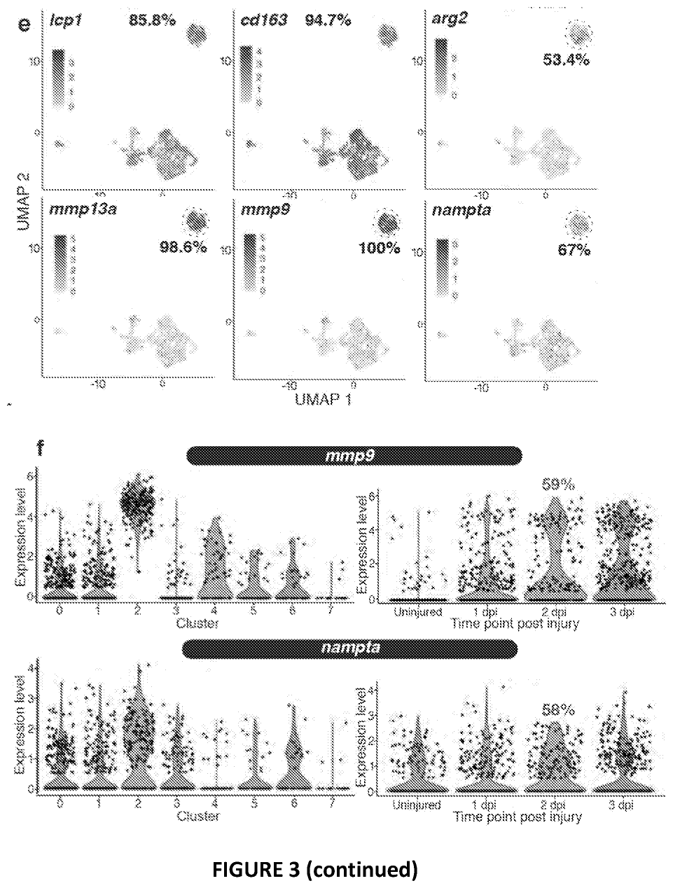

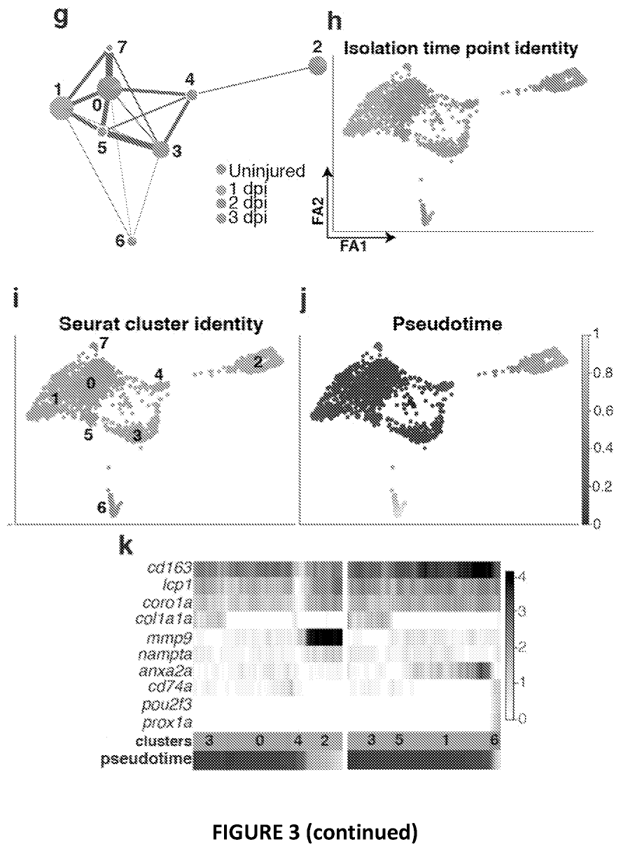

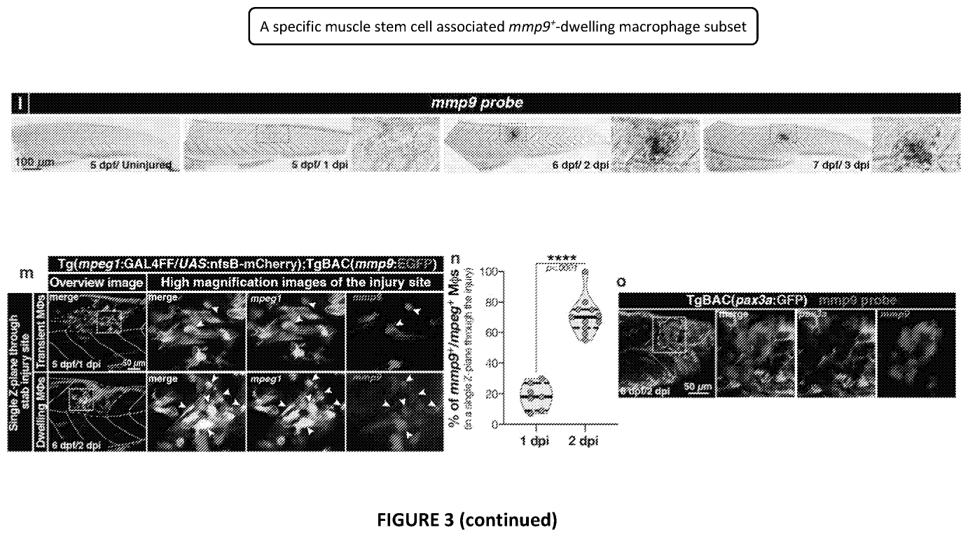

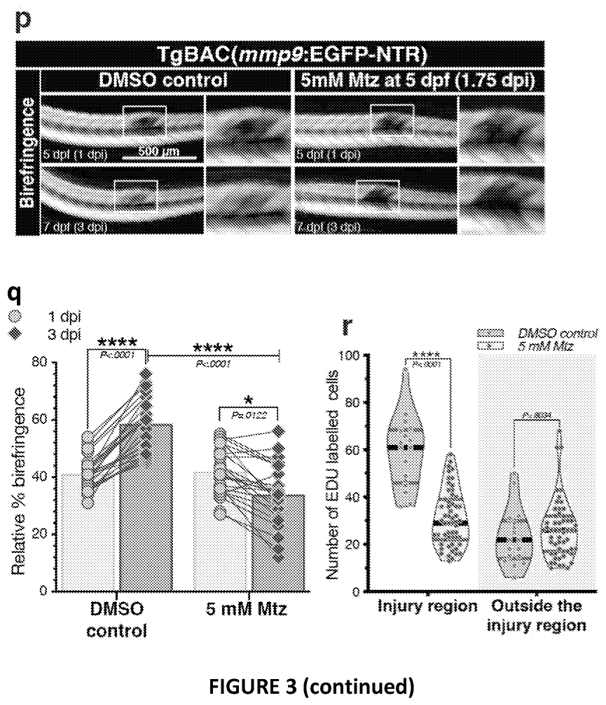

FIG. 10. NAMPT's intracellular enzymatic function does not govern its pro-proliferative function in skeletal muscle regeneration. a-b, mmp9 labels a subset of wound-dwelling macrophages. (a) Following laser ablation skeletal muscle injury (white dotted line), a subset of macrophages (Tg(mpeg1:GAL4FF/UAS:NfsB-mCherry), yellow) starts expressing mmp9 (TgBAC(mmp9:EGFP), magenta) upon dwelling (yellow/magenta double labelled cells). Following injury, mpeg⁺ macrophages respond to the wounding. At the same time, neutrophils-expressing mmp9 are also seen to be present in the injury (magenta only, green open arrowheads, exhibiting a cellular phenotype and wound site dynamics distinct from macrophages. These cells co-express the neutrophil-specific marker mpx (data not shown, n=16). These mmp9-expressing neutrophils exit the wound site by 5.21±1.27 hpi (n=6)). Once macrophages start to dwell, a subset of mpeg-expressing macrophages also start to express mmp9 (white closed arrowheads). Frames extracted from Supplementary Video, 10 (n=6). (b) Titrating the metronidazole (Mtz) dose enables specific ablation of mmp9-expressing immune cells in the TgBAC(mmp9:EGF-NTR) (magenta) transgenic line. Upon 5 mM Mtz treatment for 6 h (1.75 dpi (5 dpf) to 2 dpi (6 dpf)) uninjured larvae show ablation of mmp9-expressing immune cells (phenotypically distinct, stellate morphology, mmp9 high expressing cells), while skin cells, which express mnp9 at significantly lower levels (phenotypically distinct, hexagonal morphology) are resistant to ablation. Following needle stab skeletal muscle injury (4 dpf/0 dpi), larvae treated with Mtz at 5 dpf/1.75 dpi demonstrate an absence of mmp9 high expressing cells, specifically in the wound site (dotted white line)(n=12). c-d, The dose and duration of Mtz treatment used specifically ablates 77.94% of mmp9⁺/mpeg⁺ macrophages (mmp9⁻/mpeg⁺ macrophages: 6.65±1.50 in Mtz treated vs 5.47±2.75 in control. mmp9⁺/mpeg⁺ macrophages: 2.18±1.19 in Mtz treated vs 9.87±2.72 in control). (c) Representative images, with white arrowheads identifying mmp9⁺/mpeg⁺ (magenta and yellow, respectively) macrophages within the needle stab skeletal muscle injury zone (white dotted line). (d) Quantification. e-g, Ablation of mmp9⁺ macrophages leads to a significant reduction in wound site present muscle stem/progenitor cells. Following Mtz treatment there is a significant reduction in wound site (white dotted line) located Pax7⁺ muscle stem cells (e). (f) Quantification. This reduction is not due to a stem cell migration deficit, as the Mtz treatment is carried out following muscle stem cell migration (wound edge lining muscle stem cells in the Mtz treated larvae can be visualised in, e), but rather due to a muscle stem cell proliferation deficit within the wound site (yellow dotted line) as observed by assaying EdU incorporation (g, 3 independent examples of larval injuries for both control and Mtz treated larvae are presented. Images are representative of the quantification presented in FIG. 3r). d, f, Mean±S.D. Unpaired I-test. h, nampta mRNA expression is specifically up-regulated in the injury site from 2 dpi onwards (black arrowhead, n>15) in larval zebrafish. i-j, Increased NAMPT activity leads to elevated levels of intracellular NADH. (i) NADH auto-fluorescence (magenta) displays a localised up-regulation in dwelling macrophages (yellow) indicating these cells as the primary source of wound-present Nampt (n=15). (j) This was further was quantitatively confirmed by means of a bioluminescent-based assay, that demonstrated wound-located dwelling MΦs having a lower NAD⁺/NADH ratio when compared to transient MΦs. Mean±S.D. Unpaired t-test. k-o, Assessing Nampt expression in larval zebrafish. Nampt antibody staining carried out in a developmental series (1, 3 and 4 dpf) confirmed a match between the distribution pattern of protein and previously published RNA transcript³² and demonstrated ubiquitous expression during early development, with enrichment at the somite boundaries (white closed arrowheads) at 1-4 dpf and further enrichment at the intestine (white open arrowhead) from 3 dpf onwards (n=10) (k). (1-m) Following needle stab skeletal muscle injury, wound site Nampt protein expression is up-regulated starting at 6 dpf/2 dpi. Quantification (m). Mean±S.D. Unpaired t-test. (n-o) Nampt up-regulation in the wound site (white dotted line) is of macrophage origin, as selectively-ablating macrophages (5 mM Mtz added at point of injury (4 dpf/0 dpi) to Tg(mpeg1:GAL4FF/UAS:NfsB-mCherry) larvae to ablate all macrophages) leads to a significant reduction in wound site Nampt expression. This is further affirmed, by Nampt levels being unperturbed upon selective ablation of neutrophils (Tg(mpx:KALTA4/UAS:NfsB-mCherry) larvae utilised in neutrophil ablation). Quantification (o). Mean±S.D. Two-way ANOVA with Tukey's multiple comparison test. p-q, Inhibiting Nampt's enzymatic function does not affect muscle stem cell proliferation. Following needle stab skeletal muscle injury (white dotted line) TgBAC(pax3a:GFP) (cyan) larvae were treated with the small molecular competitive inhibitor, GMX1778 from 5 dpf/1.75 dpi till experimental end point (6 dpf/2.5-2.75 dpi). GMX1778 selectively inhibits Nampt's rate limiting enzymatic function in NAD⁺ biosynthesis and at the administered concentration results in a severe reduction in larval NAD⁺/NADH levels (see FIG. 10c). Inhibiting Nampt's enzymatic function had no effect on muscle stem cell proliferation in the injury zone (p), highlighting that Nampt's functionality during stem cell proliferation is distinct to its intracellular role in energy metabolism. Quantification (q). Mean±S.D. Two-way ANOVA with Tukey's multiple comparison test.

FIG. 11. NAMPT binds to the CCR5 receptor present on muscle stem cells and induces proliferation. a-b, NAMPT selectively binds to CCR5. (a) ELISA plates were coated with human recombinant CCR5 (hrCCR5) or BSA and further incubated with human recombinant NAMPT₍₁₎ (hrNAMPT₍₁₎) at increasing concentration (0 nM to 800 nM). NAMPT molecules bound to CCR5 were detected via a biotinylated antibody. NAMPT binds to CCR5 with a K_{D} of 172±18 nM (n=2 independent experiments in triplicate). The graph shows a representative binding curve where non-specific binding to BSA was deducted. Means±S.E.M. (b) ELISA plates were coated with mouse recombinant CCR5 (mrCCR5) or BSA and incubated with mouse recombinant CCL4 (mrCCL4) at increasing concentration (0 nM to 400 nM) along with 100 nM hrNAMPT₍₁₎. hrNAMPT₍₁₎ molecules bound to CCR5 were detected via a biotinylated antibody. mrCCL4 shows an IC₅₀ of 34.4±2.2 nM (n=2 independent experiments in triplicate). The graph shows a combined binding curve where non-specific binding to BSA was deducted. Data are means±SEM. c, Cultured Maf/DKO macrophages actively secrete NAMPT. ELISA-quantified NAMPT concentration in the supernatants of 16 h cultured Maf/DKO and Raw 264.7 macrophages (n=1 experiment in triplicate, stimulated with 10 ng/ml M-CSF). Mean±S.D. Unpaired t-test. d-e, Cultured Maf/DKO macrophages do not actively secrete CCR5's cognate ligands, CCL3, CCL4 and CCL5. (d) Representative array spots detecting CCR5 ligands expressed by Maf/DKO macrophages following 16 h stimulation with M-CSF (10 ng/ml). (e) Quantification. Mean±S.E.M. (n=1 array per group in duplicate). f, Exogenous NAMPT supplementation enhances myoblast proliferation. In vitro assay assessing the effects of exogenously introduced factors on C2C12 myoblast proliferation. Proliferation is identified by EdU incorporation. NAMPT administration (2 commercially available NAMPT sources tested, hrNAMPT₍₁₎ and hrNAMPT₍₂₎) leads to a dose dependent increase in myoblast proliferation. This effect is specifically mediated via the CCR5 receptor. Co-administration of NAMPT with the CCR2/CCR5 dual inhibitor cenicriviroc (CVC) and CCR5 specific inhibitor maraviroc (MVC) abolishes NAMPT's pro-proliferative response, while co-administration with the CCR2 inhibitor PF-4136309 (PF) does not hinder NAMPT's stimulatory effect on myoblast proliferation. In agreement with this finding, CCR5's endogenous ligands mrCCL8 and mrCCL4 functioned to enhance C2C12 proliferation while the CCR2-specific ligand mrCCL2 failed to increase proliferative rates beyond that of the control. NAMPT's pro-proliferative function is separate from its intracellular role in energy metabolism, as co-administering NAMPT with a NAMPT enzymatic inhibitor GMX1778 does not impact its effect on myoblast proliferation. Mean±S.D. Two-way ANOVA with Tukey's multiple comparison test. g, NAMPT stimulates proliferation of PAX7⁺ satellite cells in mouse primary myoblast monocultures. This pro-proliferative response is abolished upon administration of CVC. Co-culturing myoblasts with macrophages stimulates satellite cell proliferation, a response that is inhibited upon CVC administration. Co-culturing myoblasts with 3T3 cells that do not secrete NAMPT, does not stimulate satellite cell proliferation. Mean±S.D. Two-way ANOVA with Tukey's multiple comparison test. h-p, Inhibiting Ccr5 receptor activation does not affect injury-responsive immune cell dynamics in larval zebrafish. (h-i) The predicted orthologous Ccr5 gene in zebrafish was identified by determining the appropriate best hit for the human CCR5 amino acid sequence in the zebrafish genome using BLAST. A maximum-likelihood phylogenetic tree constructed using protein sequences of Ccr5 positioned the putative zebrafish Ccr5-like (Ccr5L) sequence as a homolog of mammalian CCR5 with strong bootstrap support. Bootstrap values with 500 replicates are documented below branches. CCR7 was used as an out-group. The accession numbers for genes included in our analysis are provided in the table (i). (j) pax3a⁺ muscle stem cells express zfCcr5 receptor at 2 dpi as detected by RT-PCR. (k-l) Ccr5 inhibition by the receptor antagonist CVC does not affect macrophage (Tg(mpeg1:GAL4FF/UAS:NfsB-mCherry), yellow) migration into the injury site (skeletal muscle labelled in Tg(actc1b:BFP), magenta) or the successful transition into a dwelling MΦ subtype. Quantification (1). (m-n) CVC treated macrophages appear morphologically indistinguishable from controls, with transient MΦs possessing lower sphericity values then their dwelling counterpart. Quantification (n). (1, n) Mean±S.D. Two-way ANOVA with Tukey's multiple comparison test. (o-p) CVC treatment does not alter the neutrophil (Tg(mpx:eGFP), magenta) response to needle stab muscle injury (white dotted line) (o). Quantification (p). Mean±S.D. Unpaired t-test. q, CVC treatment (pre-treatment for 2 h and maintained following laser ablation skeletal muscle injury (white dotted line) until experimental end point) does not interfere with the initiation and maintenance of dwelling macrophage (white arrows) muscle stem cell (white arrowheads) associations in the wound site. Frames extracted from Supplementary Video. 13. r-s, Larvae soaked in CVC or MVC displayed a marked regeneration deficit revealed by birefringence imaging (r) and quantified (s). t-u, pax3a⁺ myogenic stem cell (TgBAC(pax3a:GFP), cyan) proliferation is inhibited by CVC addition as demonstrated by decreased EdU incorporation (magenta) of these cells in the wound site (white dotted line) post-injury (t) and quantified (u). s, u, Mean±S.D. Two-way ANOVA with Tukey's multiple comparison test.

FIG. 12. Zebrafish germline nampta and ccr5 mutants present severe skeletal muscle regeneration deficits in response to acute muscle injury. a-j, CRISPR/Cas9 was utilised to target exon 2 of nampta, which resulted in a germline deletion-insertion mutation which produced an altered amino acid sequence and a subsequent premature stop codon (asterisks)((nampt p.Try61Profs*4, referred to as nampta^{pc41}). (a) Sanger trace of the genotyping PCR amplicon demonstrating the effect of Cas9/gRNA induced mutation at a DNA and amino acid sequence (AA) level in the target nampta locus. (b) Schematic representation of the nampta gene highlighting its exons, mutation site, transcript and primer binding sites for reverse transcriptase (RT) PCR. (c) RT-PCR for nampt cDNA demonstrates a reduction in the level of mutant transcript, encoding a truncated protein, highlighting that it is targeted for degradation by nonsense-mediated decay. actc1b transcript levels act as a loading control. (d-f) Macrophage and stem cell dynamics are unaffected in the nampta mutant. (d) Both nampta heterozygous and homozygous mutants present wound site (white dotted line) macrophage (yellow) dynamics comparable to wild-type siblings, with macrophages transition to a dwelling state at 2 dpi. (e) Quantification. (f) Furthermore, dwelling macrophages in the heterozygous and homozygous nampta mutants go onto interact with wound site present Pax7⁺ muscle stem cells. Representative of n=20 observations. Homozygous mutants present a severe regeneration deficit following needle stab muscle injury as observed by birefringence imaging (g) and quantified in (h). This repair deficit could be correlated to a significant proliferation deficit (EdU, white) observed within the injury site following needle stab skeletal muscle injury (Myosin Heavy Chain (MyHC) to visualise skeletal muscle, magenta) (i). Proliferation within the injury zone in mutant larvae decreased to homeostatic levels observed external to the wound site, highlighting that the mutants failed to elicit the additional proliferative response needed to sustain repair. Furthermore, these observations recapitulate what was seen following dwelling and mmp9-expressing macrophage ablations. Quantification (j). k-t, CRISPR/Cas9 was also used to target exon 2 of ccr5 (utilising two gRNAs) resulting in a deletion-insertion mutation which induced a frame shift and subsequent premature stop codon (ccr5 p.Pro24Leufs*28, referred to as ccr5^{pc42}) (k). (1) Schematic of the two exons of ccr5, along with site of mutation (513 bp deletion and 90 bp insertion) and mRNA transcript. The primer-binding sites for RT-PCR are also documented. (m) RT-PCR analysis for the ccr5 cDNA demonstrates a 234 bp product, corresponding to the mutant transcript (red arrowhead). No wild-type transcript corresponding to a 657 bp product (red arrow) is present in the mutant. Both PCR fragments have been sequence verified. The mutant transcript lacks the majority of the chemokine domain coded for by exon 2 of ccr5, and as such the mutant protein, if translated, would be non-functional as it lacks the ligand-binding site. actc1b transcript levels act as a loading control. As for the nampta mutant described above, the ccr5 mutant presented with macrophage dynamics (representative images, n and quantification, o) and macrophage-stem cell interactions (p, representative of n=20 observations) comparable to that of their wild-type siblings. Furthermore, this mutant mirrored the phenotypic defects described above for the nampta mutant and presented with a significant skeletal muscle repair deficit upon injury (q), quantification, (r), due to a wound site muscle stem cell proliferation defect (s, muscle labelled by phalloidin, quantification (t)). e, h, j, o, r, t, Two-way ANOVA with Tukey's multiple comparison test.

FIG. 13. Immune cell-specific gene editing of nampta and namptb in larval zebrafish. a-d, Validating the macrophage-specific nampta gene editing strategy. (a) Macrophages were isolated by FACS at 3 dpf from nampta gRNA injected mpeg1-Cas9 larvae. DNA isolated from these cells was used to generate a PCR amplicon of the region encompassing gRNA target site. Sanger sequencing of the amplicon confirmed the presence of sequence disruptions, starting from a few base pairs upstream of the PAM site. (b) Nampt protein expression in the wound site was assessed in the nampta gRNA injected mpeg1-Cas9 larvae following needle stab muscle injury (white dotted line). The gene-edited larvae presented observably reduced Nampt expression (magenta) within the injury zone (n=12). c, FACS isolated macrophages from gene-edited larvae were assayed for Nampt functionality by measuring NAD⁺/NADH levels using a luminescence-based assay. Macrophages isolated from control-uninjected larvae were used to measure the baseline NAD⁺/NADH levels for larval zebrafish macrophages. Macrophages from larvae treated with the Nampt enzymatic inhibitor GMX1778 were used to identify the NAD⁺/NADH levels present in macrophages in the absences of Nampt function. Furthermore, macrophages from larvae treated with NMN, the main product of Nampt's rate-limiting enzymatic reaction were used to establish the maximum threshold of the assays sensitivity. Macrophages isolated from nampta gRNA injected mpeg1-Cas9 larvae presented with a reduction in NAD⁺/NADH levels, reflective of a loss-of-function of Nampt activity within macrophages present in these larvae. This assay would also detect the residual enzymatic activity of Namptb, which would not be affected by this gene specific targeting approach. Mean±S.D. One-way ANOVA with Dunnett's post hoc test for multiple comparison test. d-f. Macrophage (yellow) dynamics of the nampta gene-edited larvae were assayed-post wounding and demonstrated no observable deviation from the control larvae (d, n=10). nampta gene-edited larval macrophages, located within the injury zone (white dotted line), transitioned to a dwelling state at 2 dpi (e, quantification, f). These dwellings macrophages go on to interact with wound-present pax3a⁺ muscle stem cells (g, representative of n=20 observations). h-j, Muscle stem cell-specific ccr5 gene editing does not impact on injury-responsive (white dotted line demarcates wound site boundaries) macrophage dynamics and the transition to a dwelling phenotype at 2 dpi (h, quantification, i). Furthermore, these ccr5 gene-edited Pax7b⁺ muscle stem cells (cyan) display phenotypically wild-type interaction with dwelling macrophages (yellow) located in the injury zone (j, representative of n=20 observations). k-n, In zebrafish, Nampta and not Namptb governs Nampt's regenerative role in muscle regeneration. (k) Lateral view of namptb expression by in situ hybridisation in the wound site demonstrates constitutive expression from 1-3 dpi in the injury site. mpeg1-Cas9 larvae injected with two gRNAs targeting namptb (schematic, l) demonstrated modest skeletal muscle regenerative abilities by birefringence imaging (m) following needle stab muscle injury. Quantification (n). o-q, Macrophages, not neutrophils, are the primary and functional source of Nampta in muscle regeneration. nampta was specifically knocked down in neutrophils (using the mpx-Cas9 line, schematic, o), the other key innate immune cell type present in the regenerate. Using this approach, no regeneration deficit was observed following needle stab muscle injury, as observed by birefringence imaging (p). Quantification (q). f, i, n, q, Mean±S.D. Two-way ANOVA with Tukey's multiple comparison test.

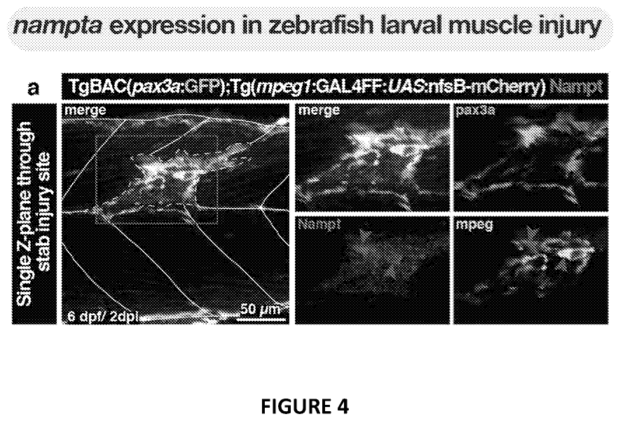

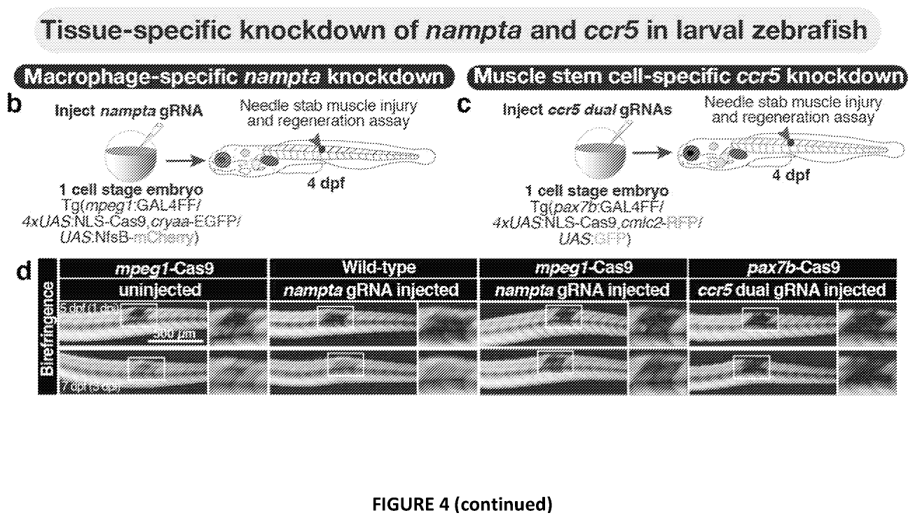

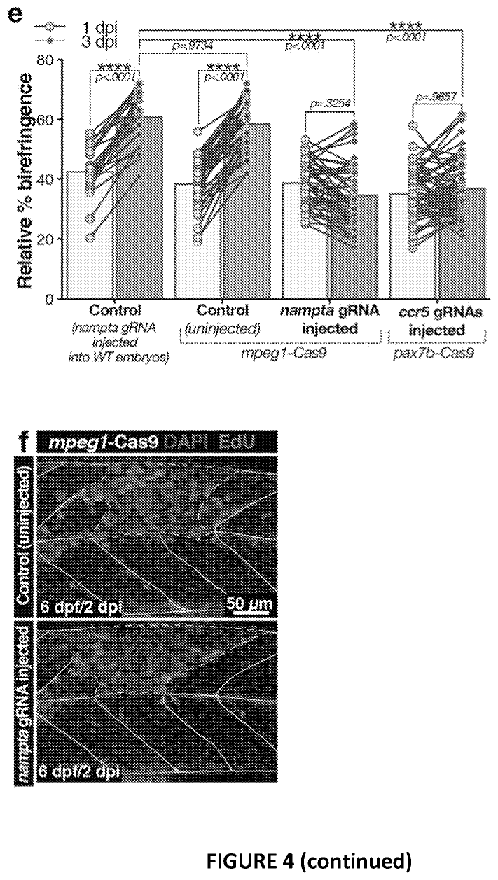

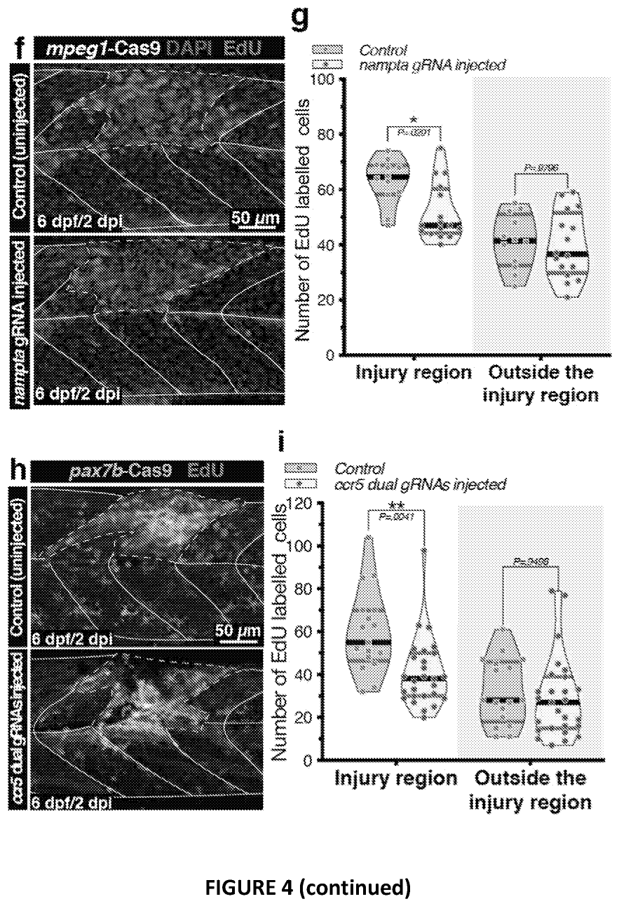

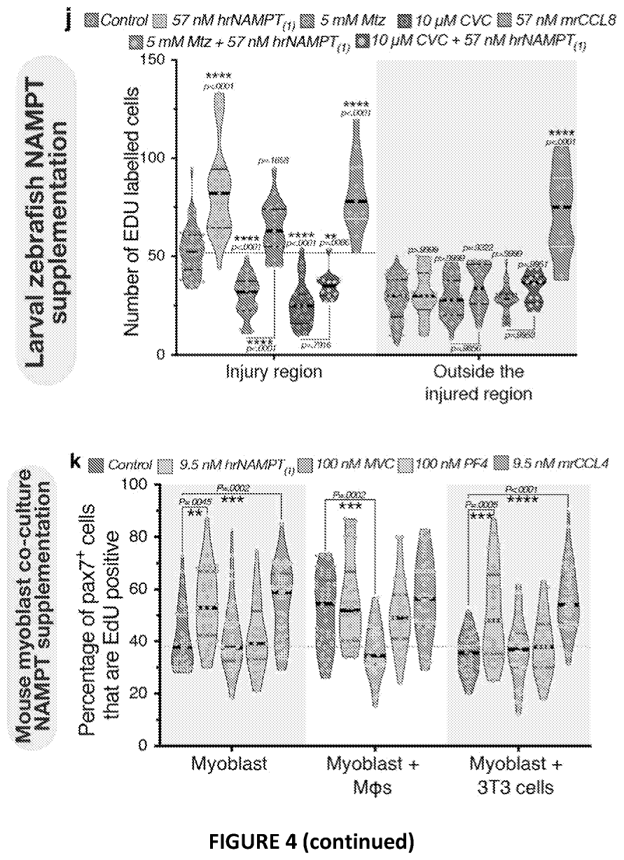

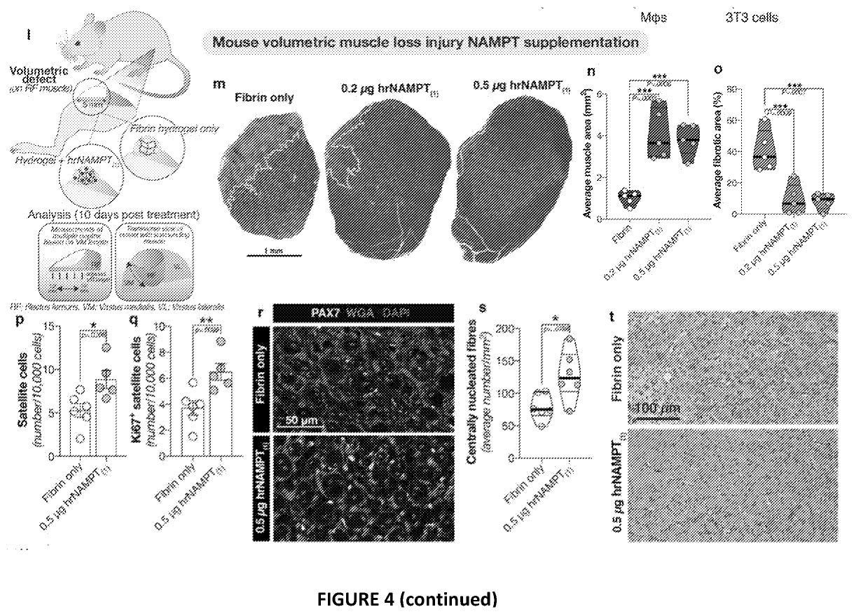

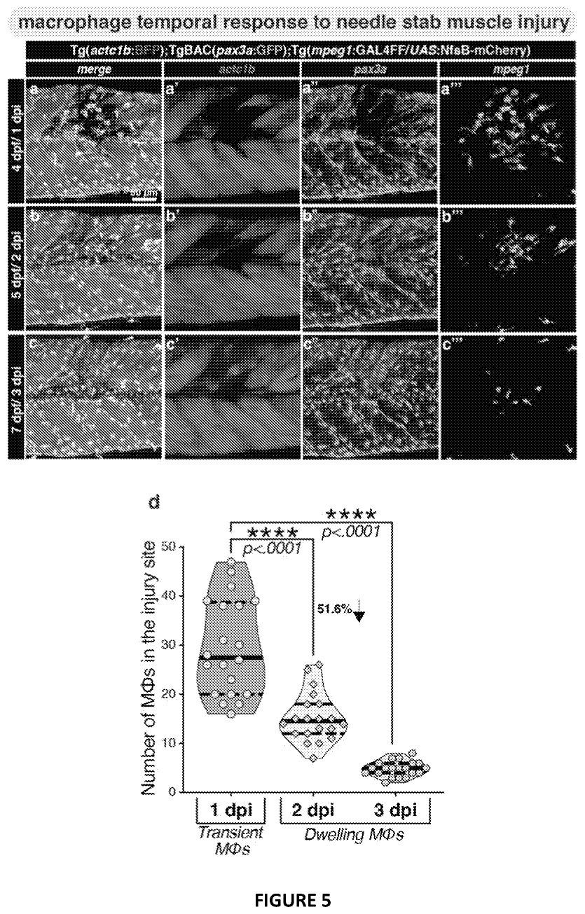

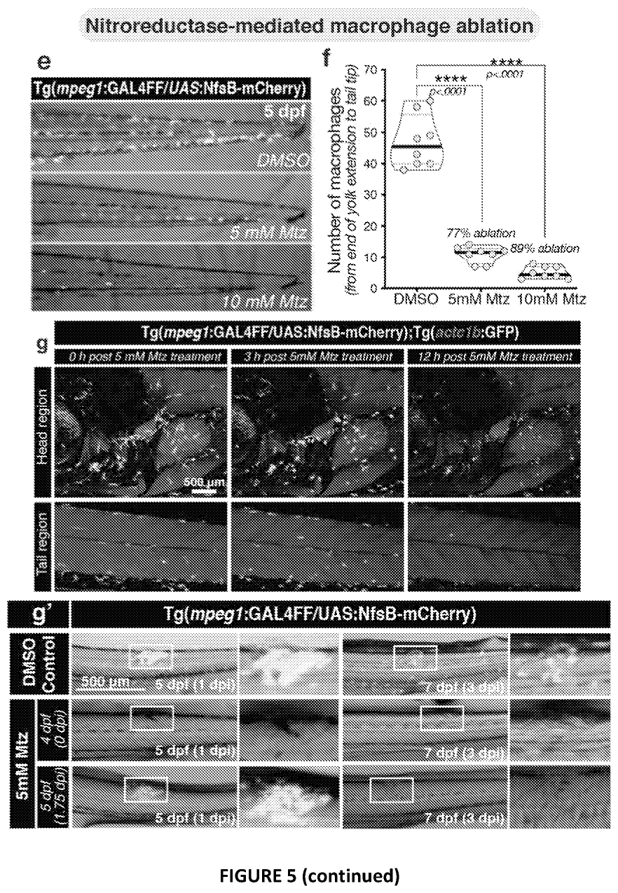

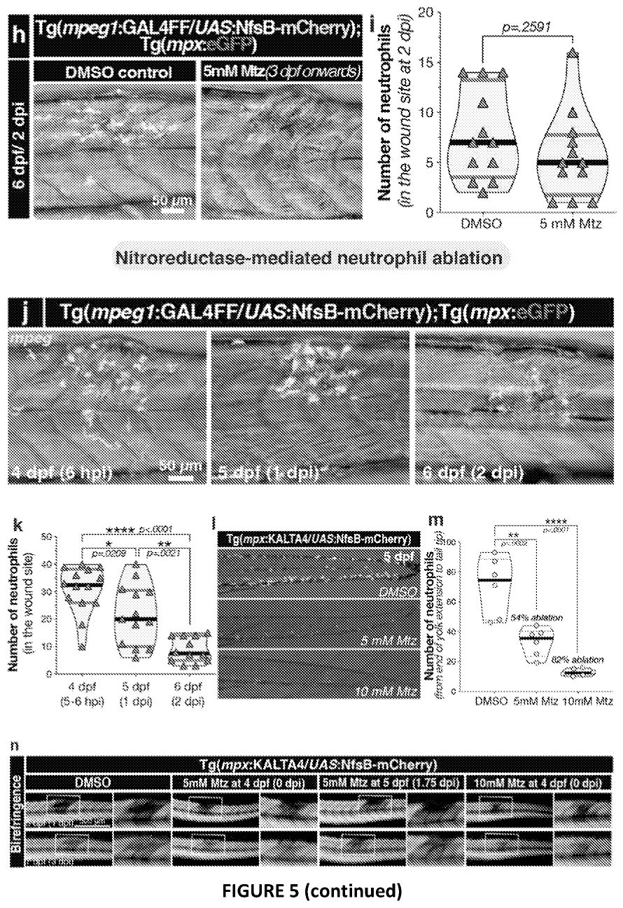

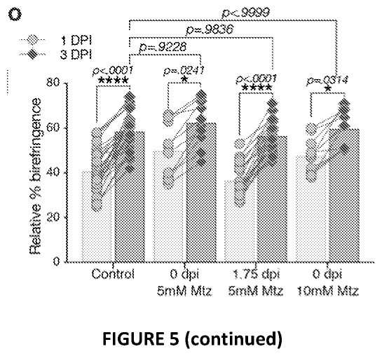

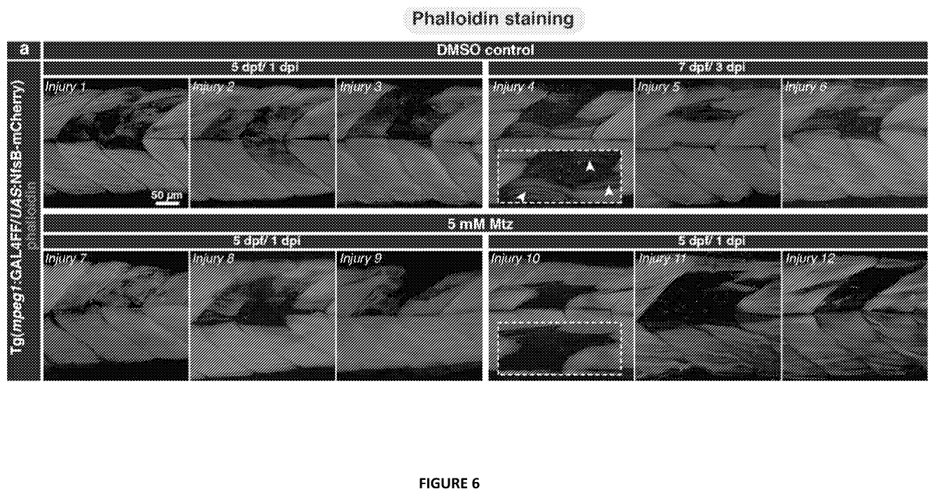

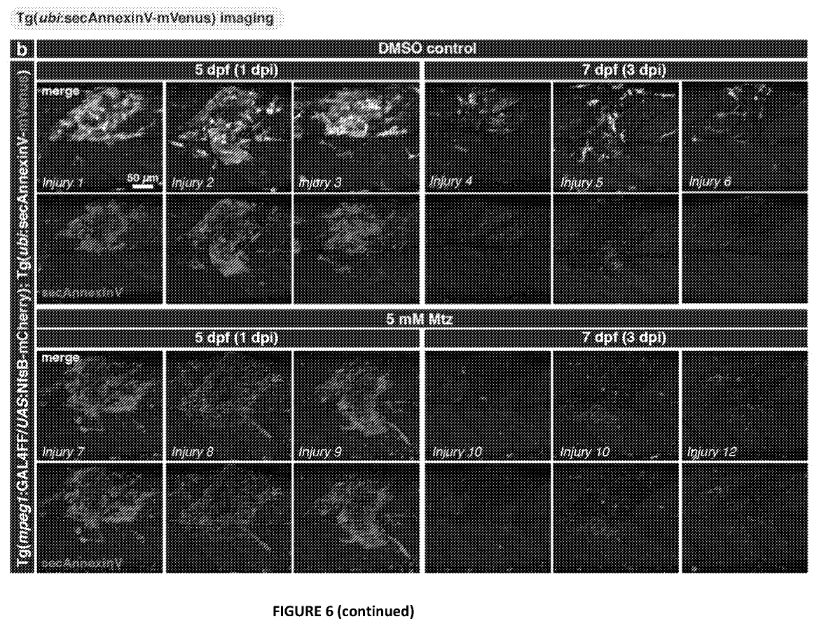

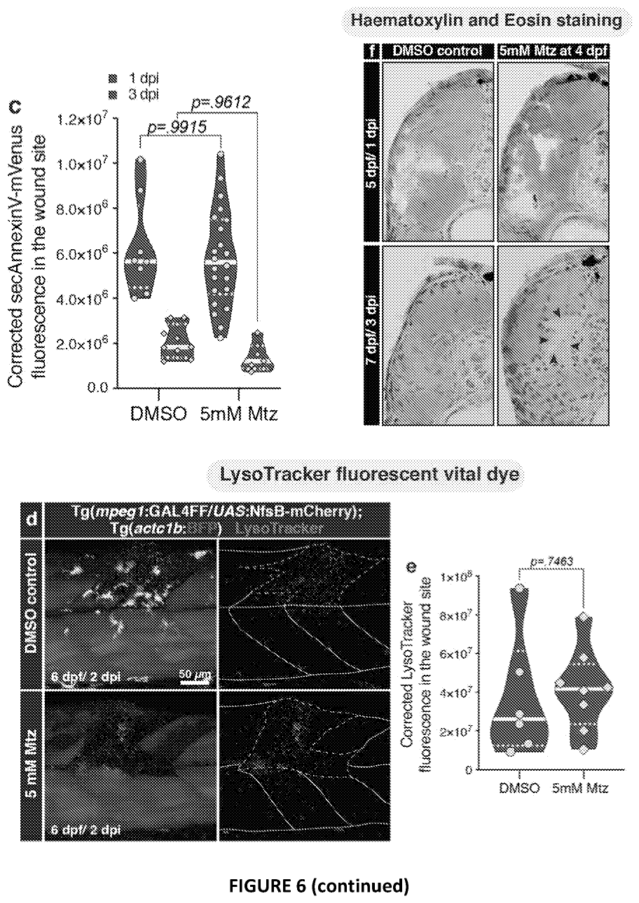

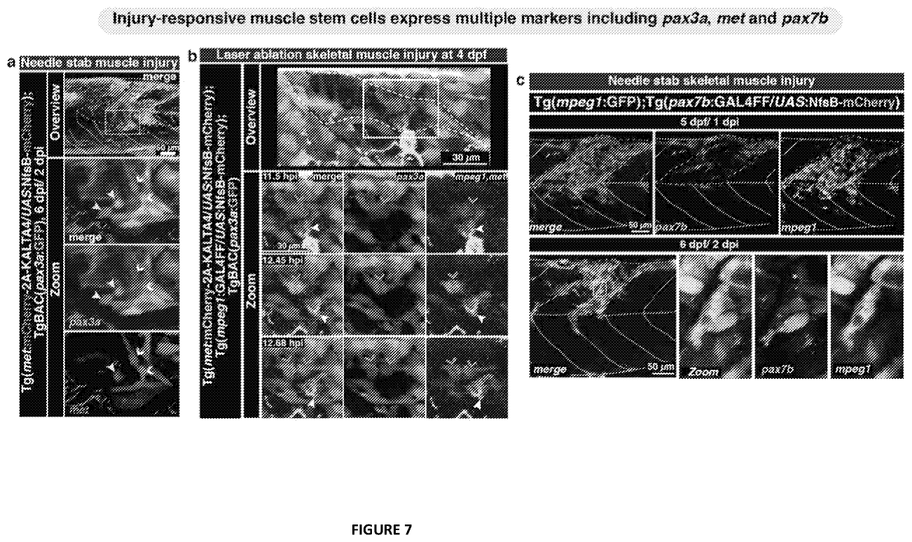

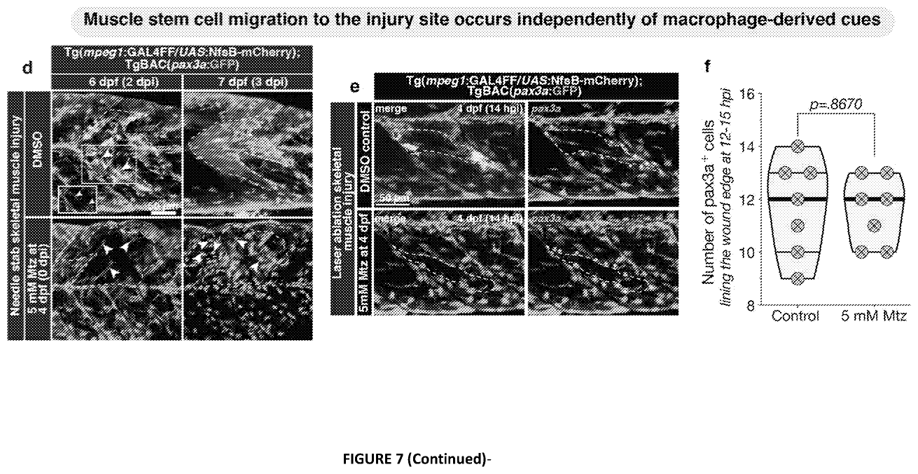

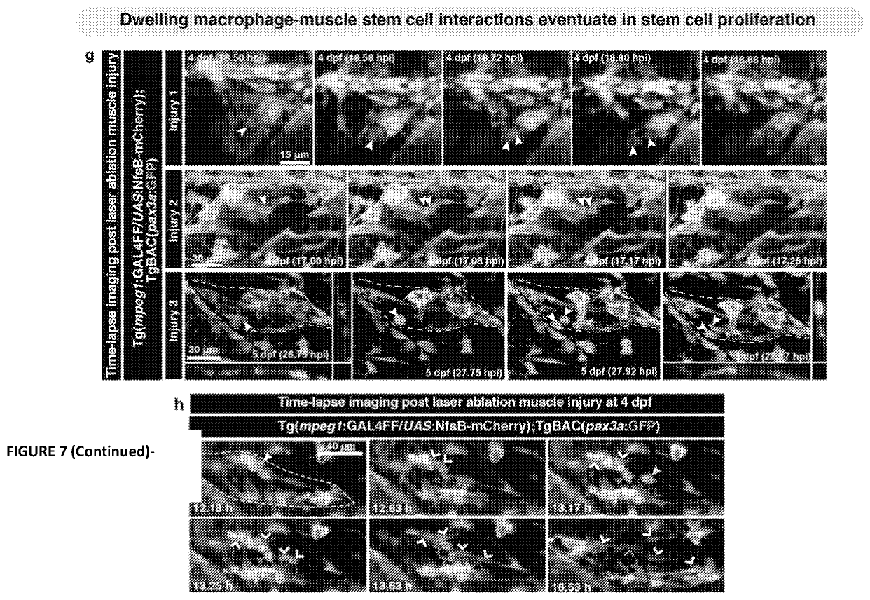

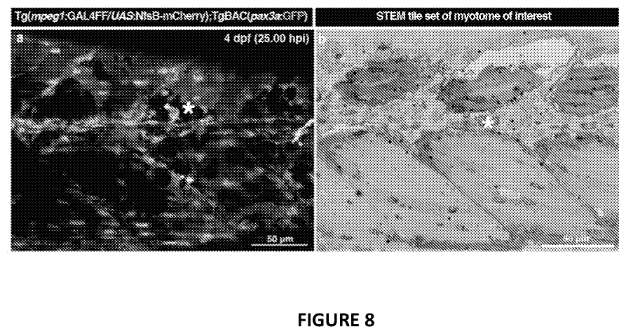

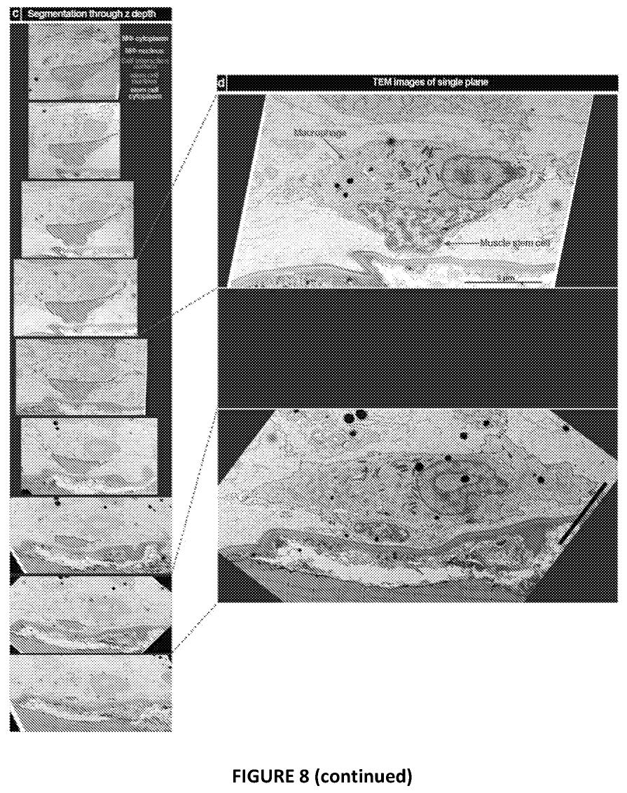

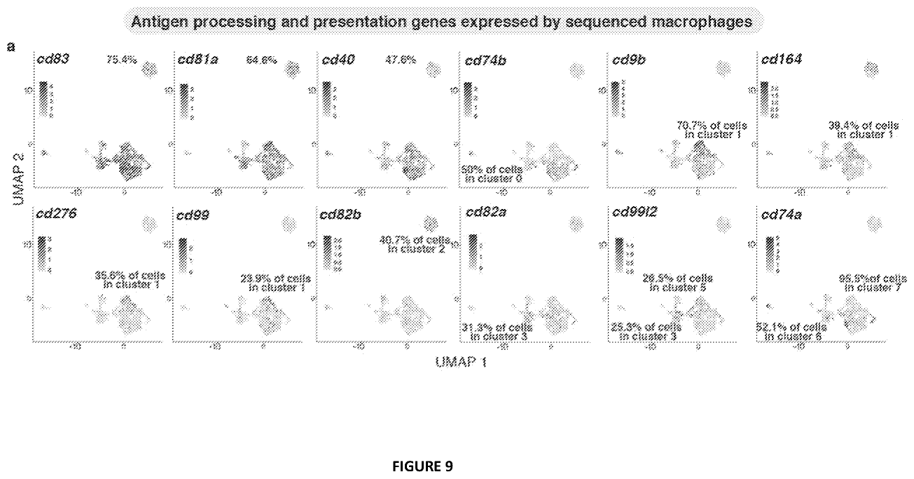

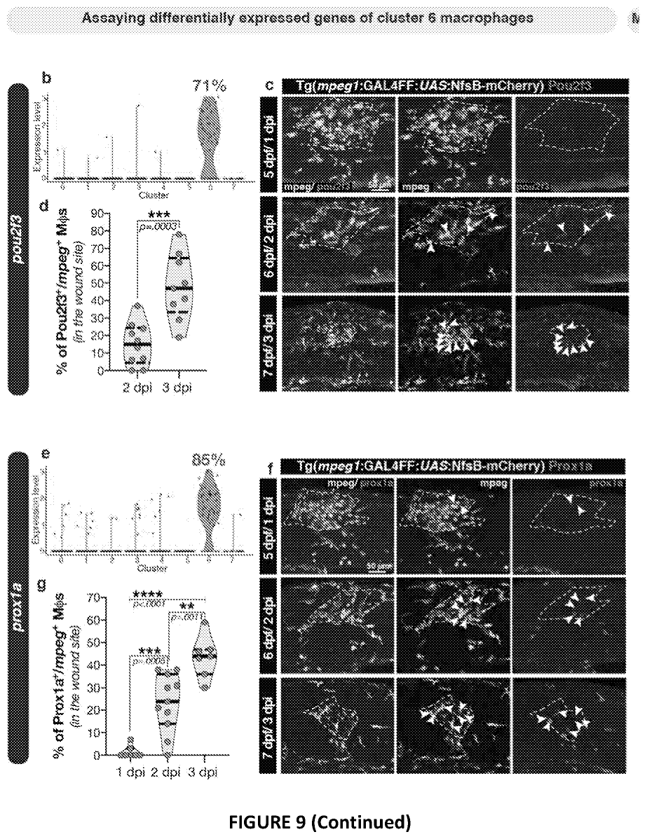

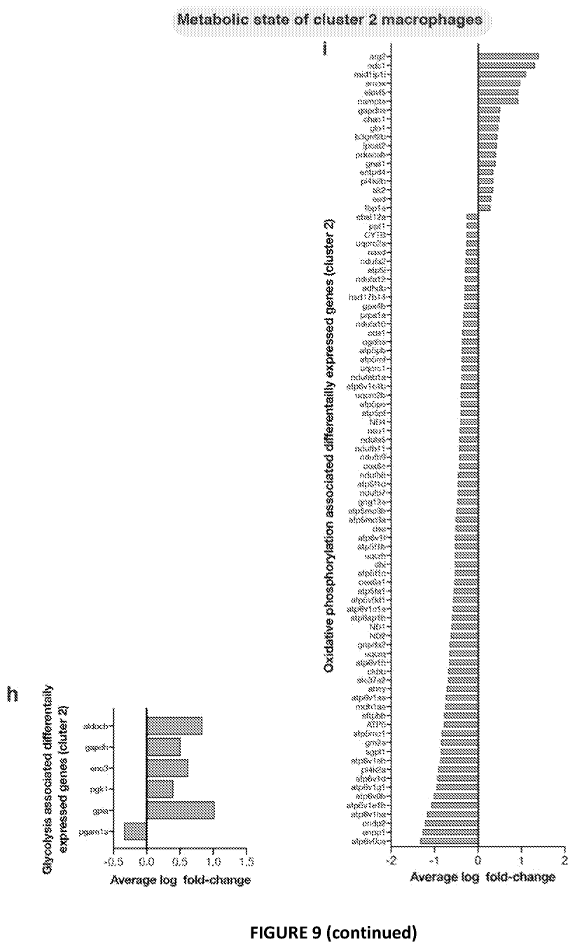

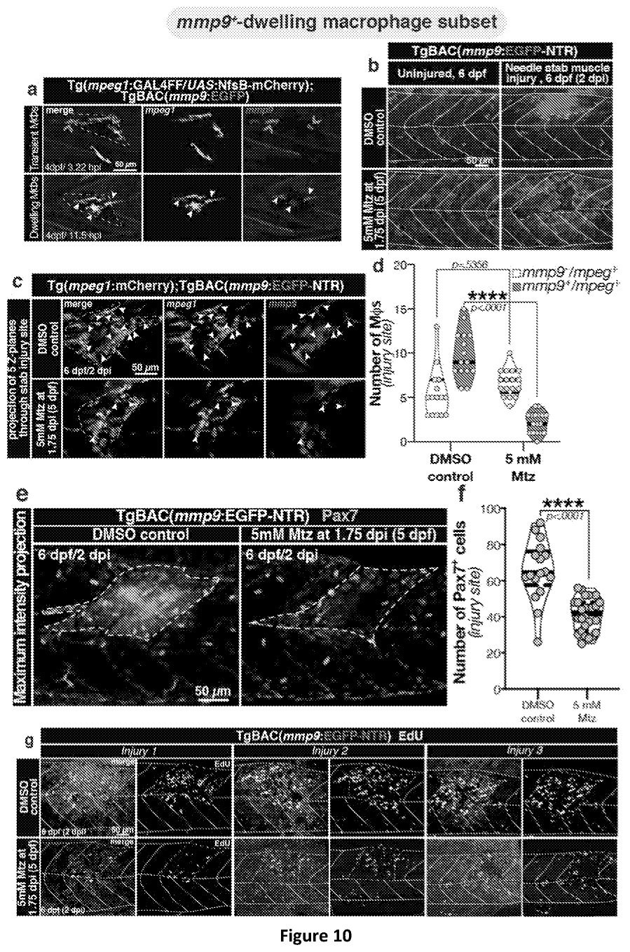

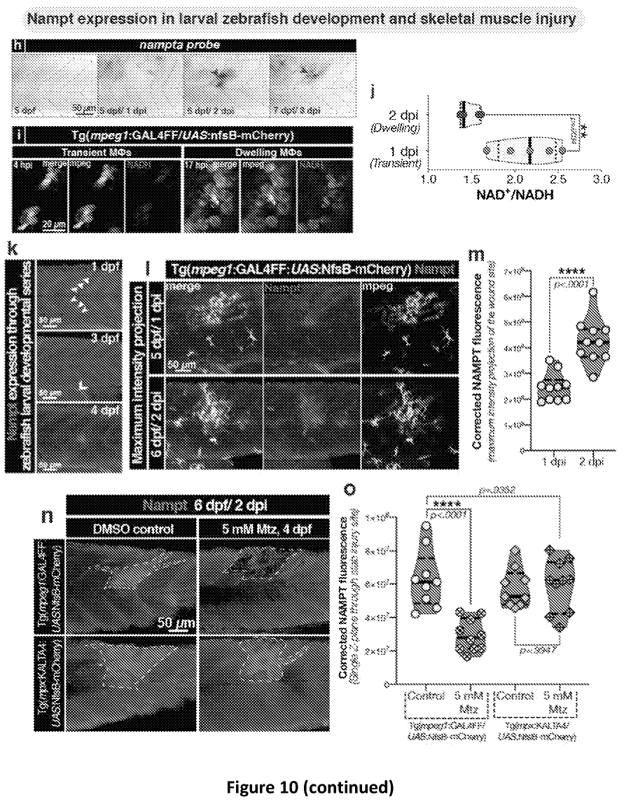

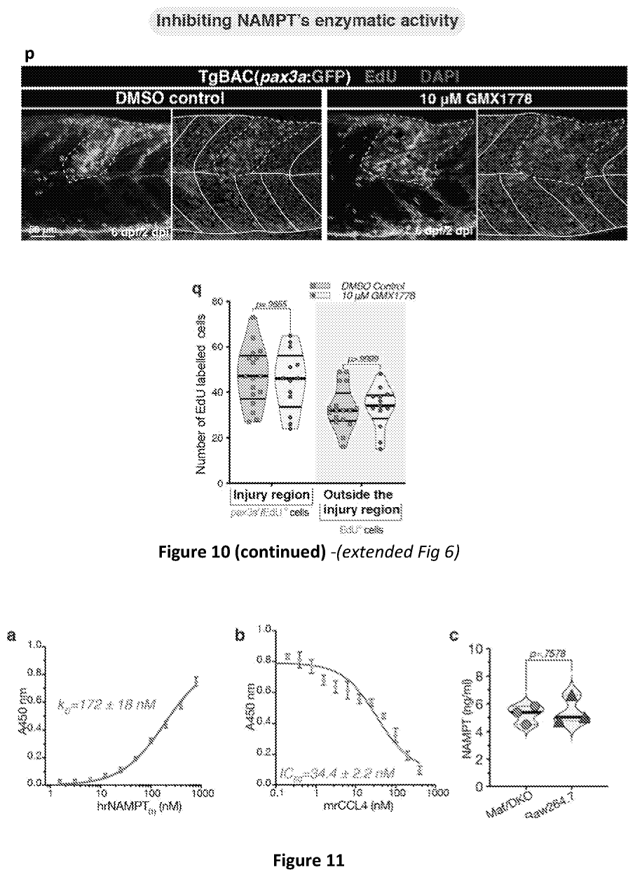

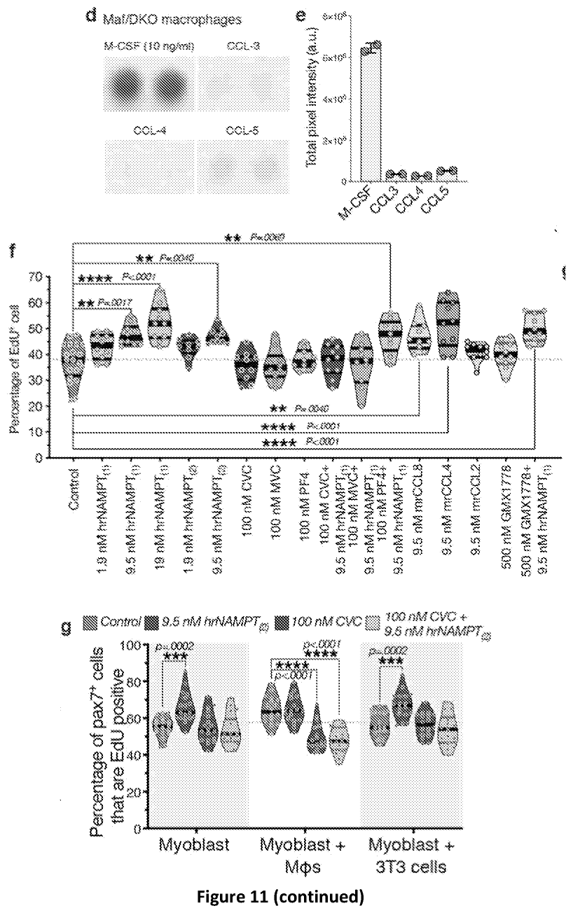

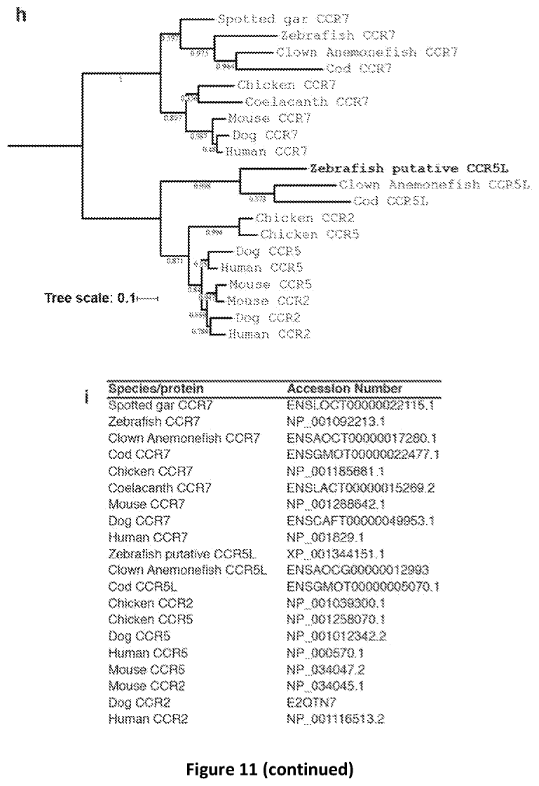

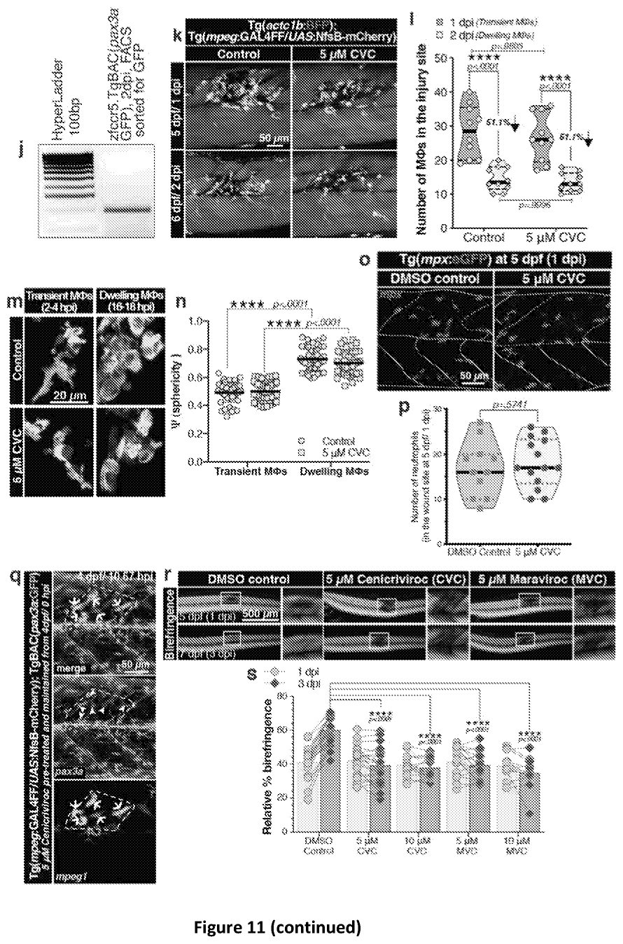

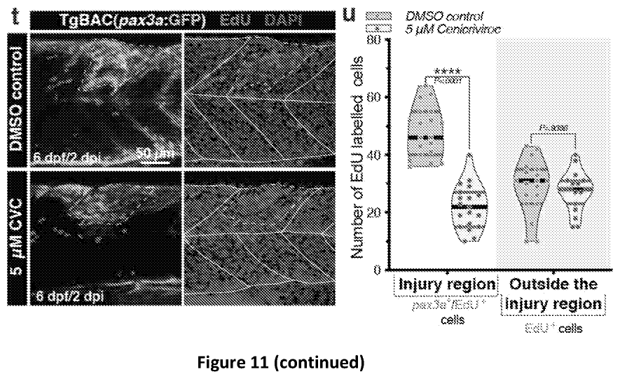

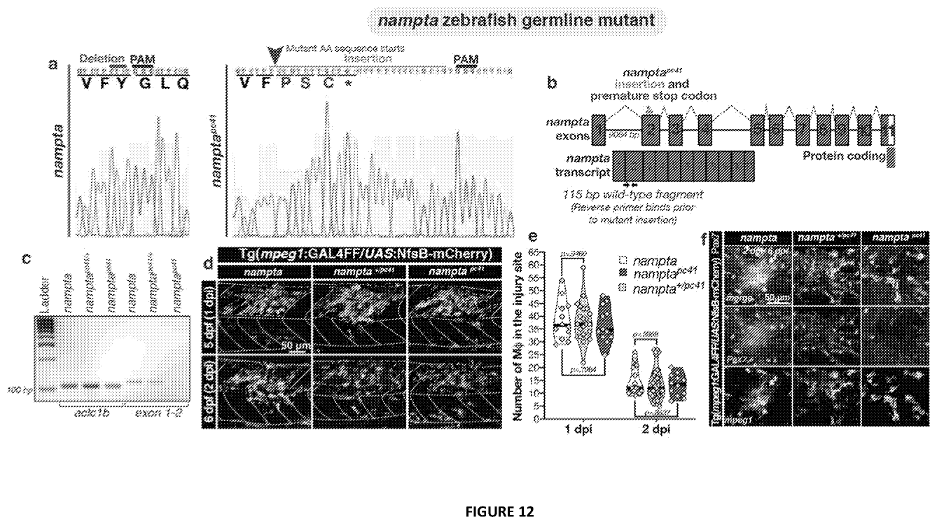

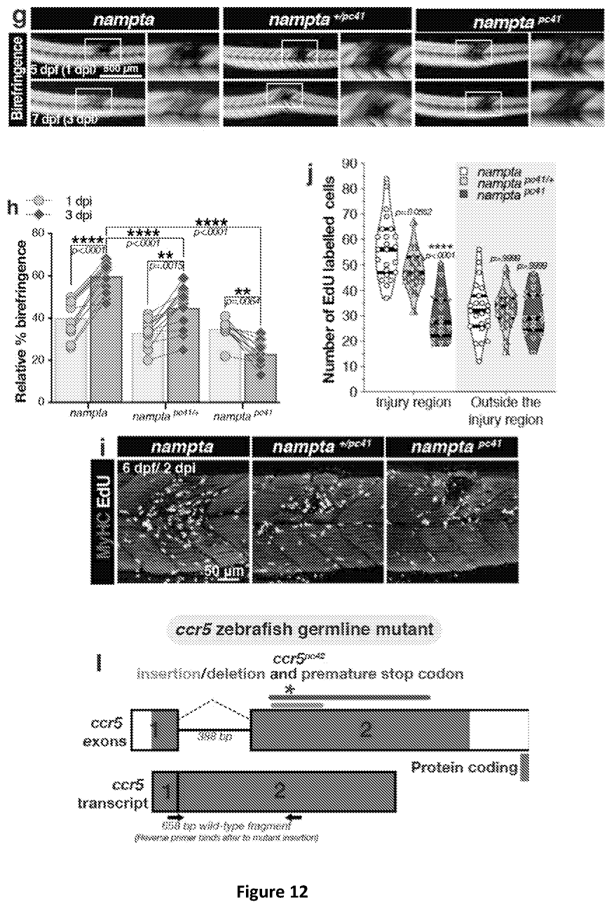

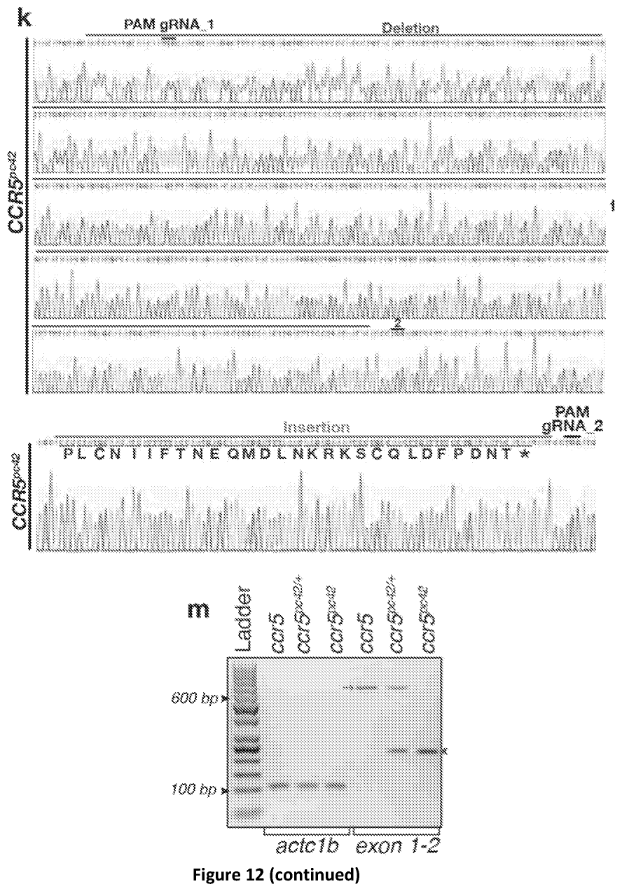

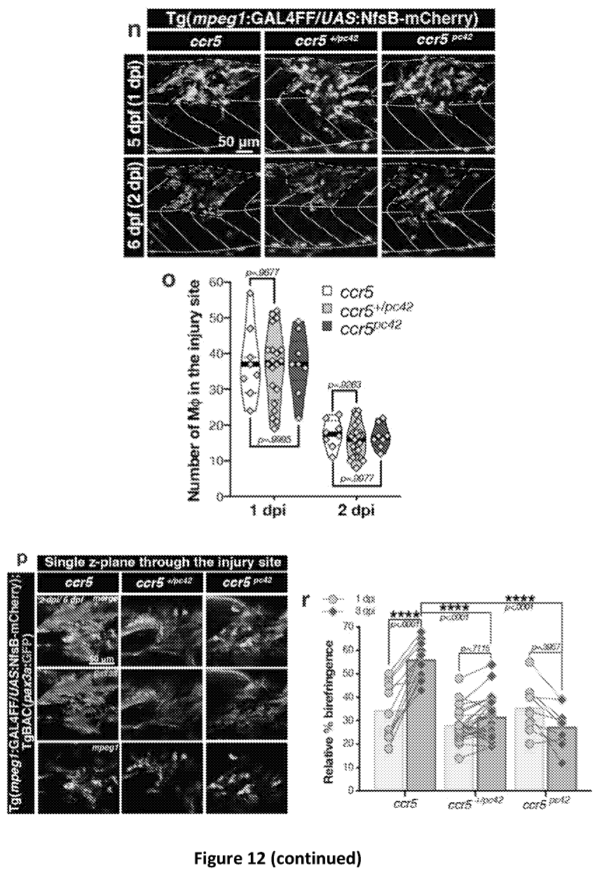

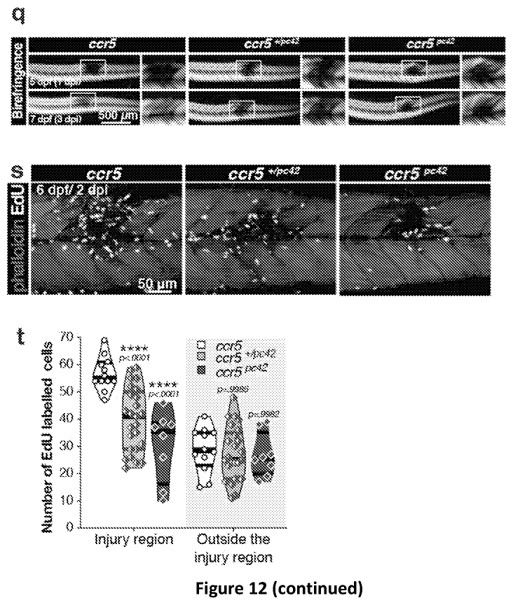

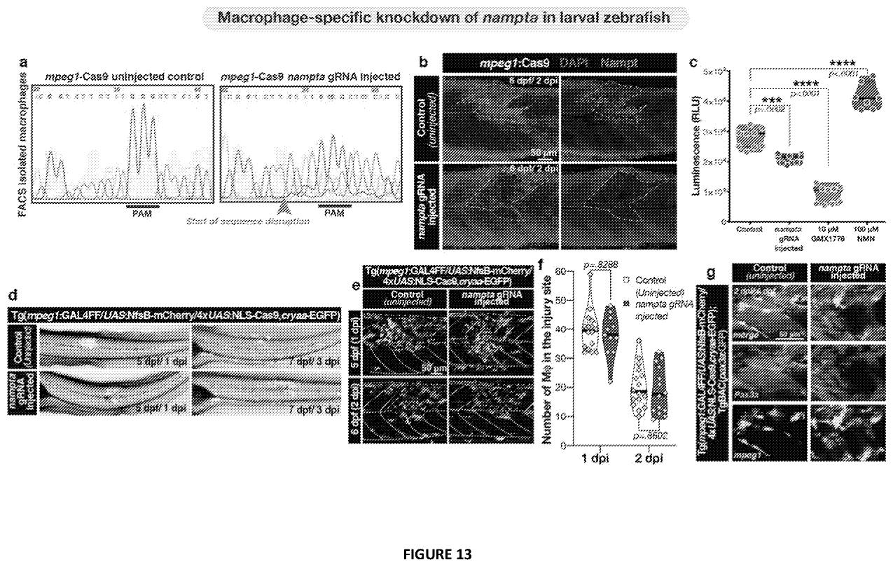

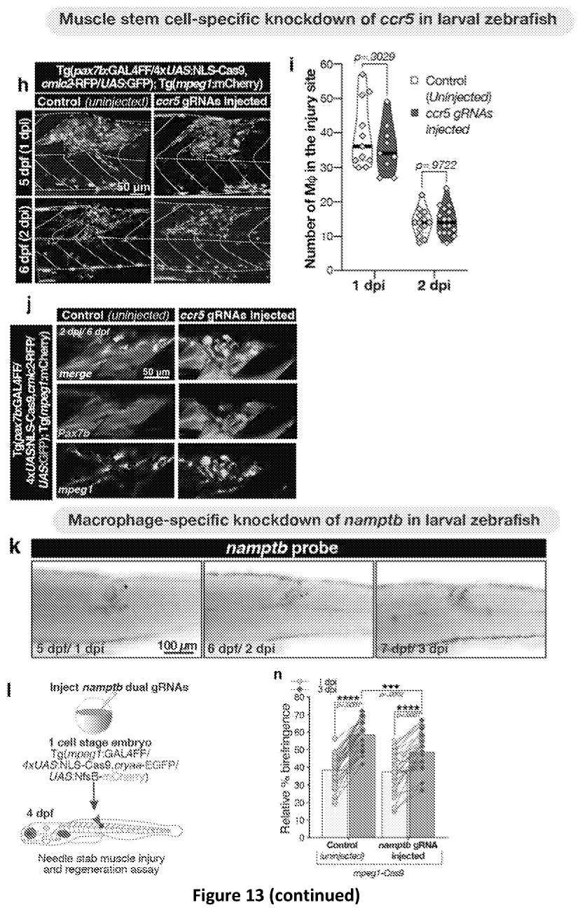

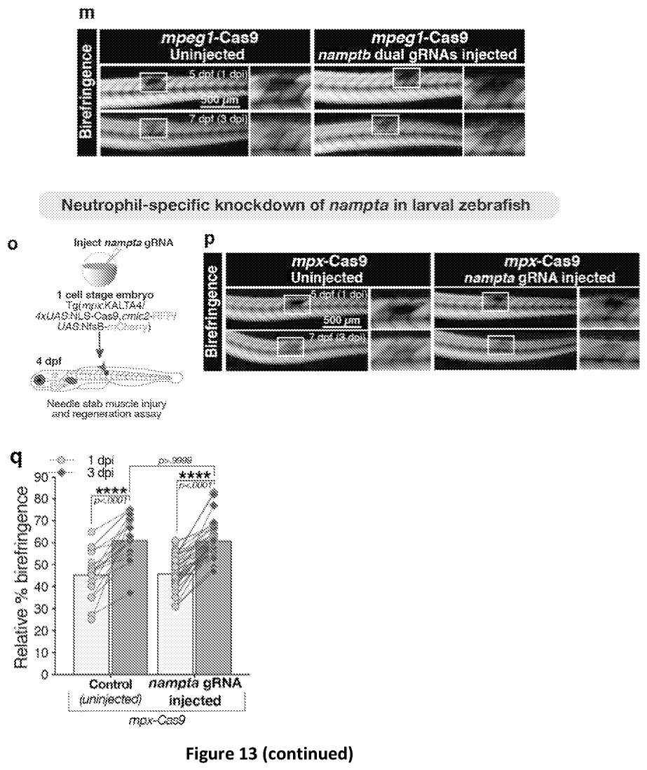

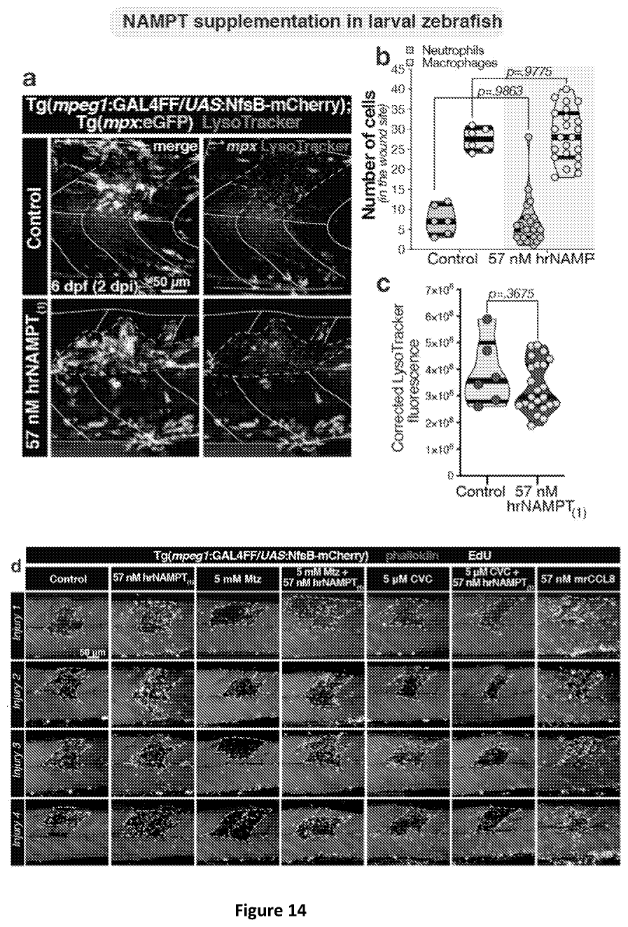

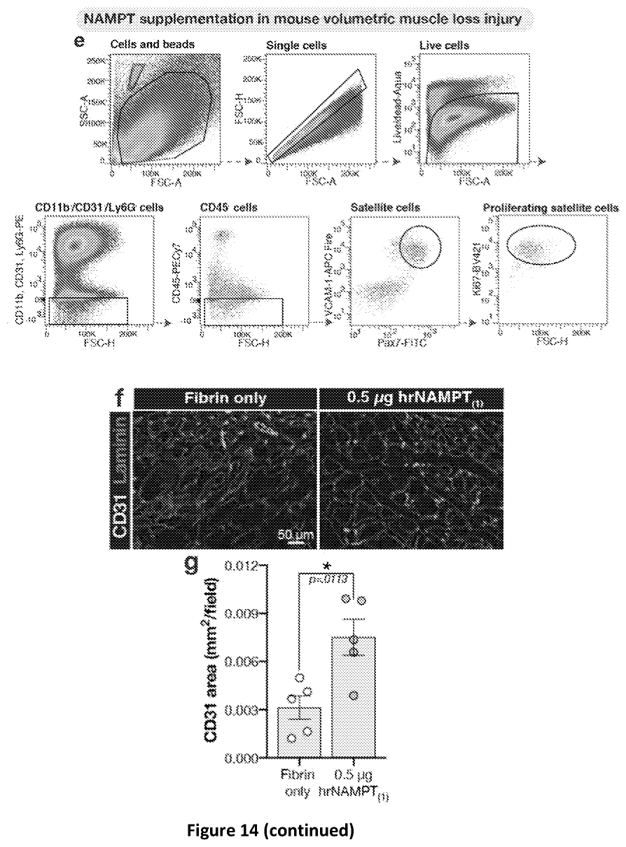

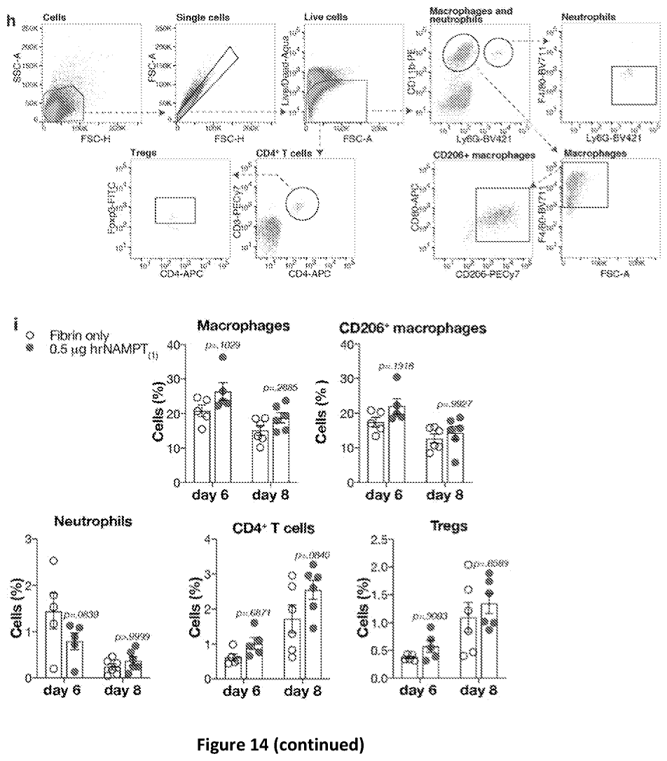

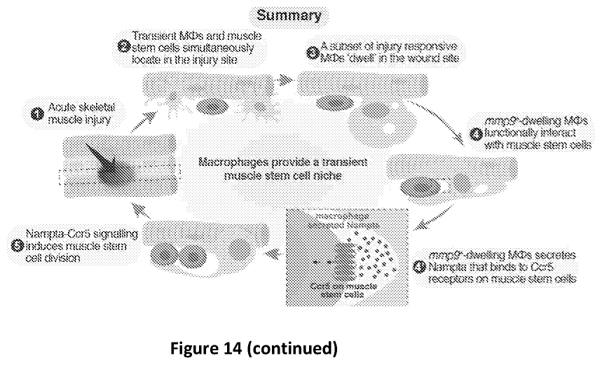

FIG. 14. NAMPT supplementation following acute skeletal muscle injury in larval zebrafish enhances proliferation, specifically in the injury zone. a-c, NAMPT supplementation does not alter the immune cell response to injury. Needle stab muscle injured larvae (white dotted line) were immediately supplemented with human recombinant NAMPT₍₁₎ (hrNAMPT₍₁₎) (4 dpf/0 dpi) and their immune cell dynamics assayed at 2 dpi. The number of macrophages (Tg(mpeg1:GAL4FF/UAS:NfsB-mCherry), yellow) and neutrophils (Tg(mpx:eGFP), cyan) were comparable to un-treated controls (a). Quantification (b). Mean±S.D. Two-way ANOVA with Tukey's multiple comparison test. Furthermore, the autophagic process within the injury zone was compared by LysoTracker (magenta) and found to be similar in control and hrNAMPT₍₁₎ supplemented larvae (a). Quantification (c). Mean±S.D. Unpaired t-test. d, The effect on wound site (white dotted line, skeletal muscle visualised by phalloidin staining, magenta) proliferation (EdU, white) was assayed following NAMPT and combination drug supplementation in the Tg(mpeg1:GAL4FF/UAS:NfsB-mCherry) line. Four individual examples are provided for each group and these images are representative of the quantification presented in FIG. 4m. NAMPT supplementation increases wound site proliferation in control settings as well as rescues the proliferative deficit that occurs upon macrophage ablation. However, as NAMPT acts on the Ccr5 receptor to elicit its proliferative response, inhibiting this receptor by CVC treatment resulted in a proliferation deficit that was resistant to NAMPT mediated rescue. Furthermore, while the Ccr5 ligand CCL8 functioned to increase proliferation in the wound site, it also increased proliferation external to the injury zone highlighting a lack of specificity that NAMPT is able to exert. e, Proliferating satellite cells in mouse muscle injuries supplemented with NAMPT. The gating strategy to isolate the proliferating satellite cell population is shown. Cells that are CD45⁻, CD11b⁻, Ly6G⁻, CD31⁻, VCAM-1⁺, and PAX7⁺ are considered as satellite cells. Proliferating satellite cells are additionally Ki67⁺. Quantification at 4 days post treatment is documented in FIG. 4 q-r. f-g, Angiogenesis following mouse VML injury treated with NAMPT. (f) Muscle injuries were treated with NAMPT (0.5 μg) delivered in fibrin or fibrin only control and tissues were harvested at 6 days post treatment. Representative regenerated muscle cryosections stained for CD31 (endothelial cells, yellow) and laminin (magenta). (g) Quantification of CD31 positive area (n=6 mice per group). h-i, Immune cell profile following mouse muscle injury supplemented with NAMPT. (h) Gating strategy to analyse wound immune cell subsets in muscle injury. The gating strategy is shown for the neutrophil, macrophage, and T cell subset panels. (i) Percentages were calculated over total live cells in the harvested tissue (n=5 mice for day 6, n=6 mice for day 8). Mean±S.E.M. Two-way ANOVA with Bonferroni post hoc test for pair-wise comparisons. j, Summary schematic of injury-responding MΦs role in modulating muscle stem cell proliferation. Following acute muscle trauma (1), macrophages and muscle stem cells migrate into the injury zone (2). About half of these injury-responsive macrophages dwell in the wound site for the duration of repair (3). A subset of mmp9⁺/mpeg1⁺-dwelling macrophages start to actively interact with wound-present pax3a⁺ muscle stem cells (4). These macrophages actively secrete Nampta that binds to Ccr5 on muscle stem cells (4′) and activates a signalling cascade that results in muscle stem cell proliferation (5).

### DETAILED DESCRIPTION OF PARTICULAR EMBODIMENTS

Unless defined otherwise, all technical and scientific terms used herein have the same meaning as commonly understood by those of ordinary skill in the art to which the invention belongs.

The term “isolated cell” as used herein refers to a cell that has been removed from an organism in which it was originally found or a descendant of such a cell. The cell may have been cultured in vitro, e.g., in the presence of other cells. Also, the cell may be destined to later be introduced into a second organism or re-introduced into the organism from which it (or the cell from which it is descended) was isolated.

The term “isolated population of cells” or the like, refers to a population of cells that has been removed and separated from a mixed or heterogeneous population of cells. In some embodiments, an isolated population is a substantially pure population of cells as compared to the heterogeneous population from which the cells were isolated or enriched.

In one embodiment, the application enables a pharmaceutical or physiologically active regenerative composition comprising one or two or three or four or five of:
(i) comprising or encoding a CCR5 interacting agent as defined herein,
(ii) a tissue stem cell (such as a satellite cell) or precursor therefore or progeny thereof
(iii) a macrophage or a precursor therefore or progeny thereof
(iv) a scaffold or retentive material
(v) a tissue delivery enhancing component.

In one embodiment, the present application provides cellular compositions comprising one or more of one or more stem cells, stromal cells, pre-satellite cells or satellite cells, pre-macrophages or macrophages or macrophage derived factors as described herein. In one embodiment, multipotent “tissue stem cell” include a pre-muscle cell or any pre-macrophage cells from which these cell may be produced in an essentially native form or modified to express heterologous or autologous factors. Similarly, the term multipotent “tissue stem cell” may include activated progeny of the tissue stem cell.

Tissue stem cells including muscle stem cells can be isolated (for ex vivo or in vitro or in vivo procedures) or induced.

A stem cell can be contacted with a media or composition comprising a CCR5 agonist for any amount of time. For example, a stem cell can be contacted with a CCR5 agonist for 12 hours, 1 day, 2 days, 3 days, 4 days, 5 days, 6 days, 1 week or more. The stem cell can be induced or stimulated to differentiate into a cell lineage selected from the group consisting of mesoderm, endoderm, ectoderm, neuronal, mesenchymal, and hematopoietic lineage.

In some embodiments, the stem cell is a human stem cell, a multipotent adult stem cell, a pluripotent adult stem cell or an embryonic stem cell.

Human adult stem cells are mitotic and typically one daughter cell remains a stem cell. Adult tissue comprises one or more resident committed progenitor or stem cells that occupy a specific niche in their tissue and actively sense and respond to their local environment. Each tissue typically has its own resident committed stem cell committed to producing progeny that differentiate into a specific range of cell types. Muscle tissue comprises satellite cells to are committed to producing myoblasts. Other well studied stem cells of this type are mesenchymal stem cells (MSC) that produce many different cell types inter alia muscle, cartilage, bone, fat, and haematopoietic stem cells (HSC) that produce all blood cells and the haematopoietic system, and neural stem cells (NSC). All tissue contains resident stem cell populations, including heart, gut and liver. Adult stem cells are typically multipotent which refers to a cell that is able to differentiate into some but not all of the cells derived from all three germ layers. Thus, a multipotent cell is a partially differentiated cell. MSC, for example can be obtained by a number of methods well known in the art. See U.S. Pat. Nos. 5,486,358; 6,387,367; and 7,592,174, and USPN 2003/0211602. MSC may be derived from bone, fat and other tissues where they reside. “Derived” from does not refer to direct derivation and merely indices where they were originally derived.

In one embodiment, the stem cell is a non-embryonic or adult multipotent stem cell.

In one embodiment the stem cell is a HSC or MSC.

Adult stem cell expressing CCR5 can be stimulated to undergo differentiation by exposure to the CCR5 interacting agent described herein. Cells are monitored to changes in expression of for example myogenic regulatory factors known in the art.

Cells may be cultured in standard media or specifically defined media.

Cell expression may be modified by techniques know in the art.

An induced or partially induced pluripotent stem cell is a convenient source of stem cells. These are derivable from a differentiated adult cell, such as human foreskin cells.

Human iPS cells can be generated by introducing specific sets of reprogramming factors into a non-pluripotent cell which can include, for example, Oct3/4, Sox family transcription factors (e.g., Sox1, Sox2, Sox3, Sox15), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. For example, the reprograming factors can be introduced into the cells using one or more plasmids, lentiviral vectors, or retroviral vectors. In some cases, the vectors integrate into the genome and can be removed after reprogramming is complete. In some cases, the vectors do not integrate (e.g., those based on a positive-strand, single-stranded RNA species derived from non-infectious (non-packaging) self-replicating Venezuelan equine encephalitis (VEE) virus, Simplicon RNA Reprogramming Kit, Millipore, SCR549 and SCR550). The Simplicon RNA replicon is a synthetic in vitro transcribed RNA expressing all four reprogramming factors (OKG-iG; Oct4, Klf4, Sox2, and Glis1) in a polycystronic transcript that is able to self-replicate for a limited number of cell divisions. Human induced pluripotent stem cells produced using the Simplicon kit are referred to as “integration-free” and “footprint-free.” Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells express alkaline phosphatase, SOX-2, OCT-4, Nanog and Tra-1-60 markers.

The terms “naive” and “primed” identify different pluripotency states of human iPS cells. Characteristics of naive and primed iPS cells are described in the art. Naive human iPS cells exhibit a pluripotency state similar to that of ES cells of the inner cell mass of a pre-implantation embryo. Such naive cells are not primed for lineage specification and commitment. Female naive iPS cells are characterized by two active X chromosomes. In culture, self-renewal of naive human iPS cells is dependent on leukemia inhibitory factor (LIF) and other inhibitors. Cultured naive human iPS cells display a clonal morphology characterized by rounded dome-shaped colonies and a lack of apico-basal polarity. Cultured naive cells can further display one or more pluripotency makers as described elsewhere herein. Under appropriate conditions, the doubling time of naive human iPS cells in culture can be between 16 and 24 hours.

Primed human iPSC express a pluripotency state similar to that of post-implantation epiblast cells. Such cells are primed for lineage specification and commitment. Female primed iPSCs are characterized by one active X chromosome and one inactive X chromosome. In culture, self-renewal of primed human iPSCs is dependent on factors such as fibroblast growth factor (FGF) and activin. Cultured primed human iPSCs display a clonal morphology characterized by an epithelial monolayer and display apico-basal polarity. Under appropriate conditions, the doubling time of primed human iPSCs in culture can be 24 hours or more depending upon the level from the adult cells from which they were derived.

Embryonic stem cells (ESC) are characteristically pluripotent i.e., they have the capacity, under different conditions, to differentiate to cell types characteristic of all three germ cell layers (endoderm, mesoderm and ectoderm). Pluripotent cells are characterized primarily by their ability to differentiate to all three germ layers. In some embodiments, a pluripotent cell is an undifferentiated cell. Pluripotent cells also have the potential to divide in vitro for more than one year or more than 30 passages.

ESC are typically the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see U.S. Pat. Nos. 5,945,577, 5,994,619, 6,235,970). Exemplary distinguishing embryonic stem cell characteristics include, without limitation, gene expression profile, proliferative capacity, differentiation capacity, karyotype, responsiveness to particular culture conditions.

In one embodiment the stem cell is adult.

In one embodiment the stem cells are autologous or heterologous to the subject.

In one embodiment, the stem cell is mammalian or human.

Macrophages and stem cells including satellite cells may be prepared using art recognised methods and as described herein and include the use of iPSC and optionally gene editing procedures.

One embodiment isolated macrophages or stem-cell derived macrophages are modified to express the truncated NAMPT peptides described herein. Generally, M2 type macrophages are selected or provided.

In one embodiment stem cells are contacted with a CCR5 interacting agent in vitro, ex vivo or in vivo as described herein to induce activation and proliferation. Stem cells treated in vitro or ex vivo may be introduced into a wound site to effect repair or administered systemically to effect regeneration of damaged tissue or to treat or improve muscle related conditions as described herein.

CCR5 agents or cells expressing same may be administered in the form of functionalized hydrogels either alone or together with cells for transplantation. Such hydrogels or similar biomaterials or scaffolds provide enhanced transplantation efficiency at the wound site.

Hydrogels may be ECM based such as fibrin based. Alternatively, hydrogels may be non-ECM based such as acrylamide based using RAFT technology (see Chiefari et al Macromol. 31:5559-5526, 1998 and Fairbanks et al Advanced Drug Delivery Reviews 91: 141-152, 2015). Suitable materials regulate release kinetics, and have desired mechanical and physical properties for tissue regeneration as known in the art.

In one embodiment, satellite cells are encapsulated in CCR5 functionalised hydrogels or other biomaterials.

Kits comprising the cellular compositions and/or agents described herein are also provided. Kits suitable for muscle repair or regeneration are specifically contemplated. The CCR5 agonist can be pre-formulated for administration or ingredients for formulation can be provided with the kit. The CCR5 agonist is for example formulated in a hydrogel or other supporting vehicle for topical application. The CCR5 agonist may be, for example, lyophilised or liquid.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones.

Proteins are said to have an “N-terminus’ be “N-terminal” and to have a “C-terminus” or be “C-terminal.” The term “N-terminus” relates to the start of a protein or polypeptide, terminated by an amino acid with a free amine group (—NH2). The term “C-terminus” relates to the end of an amino acid chain (protein or polypeptide), which is in nature terminated by a free carboxyl group (—COOH). In the present application reference to C-terminal and N-terminal fragments broadly describe the region of the full length molecule from which the elected part is derived and it excludes a full length or a native molecule. A C-terminal fragments does not have to but may include all the C-terminal amino acids and N-terminal fragments do not have to but may include all the N-terminal amino acids.

The application discloses and enables the use of a range of CCR5 interacting agents based upon the initial findings described in the examples. In particular peptide CCR5 interacting agents are provided in the form of NAMPT and functional C-terminal fragments thereof.

A number of peptide modifications are known in the art to stabilise peptides against serum proteases or to promote intracellular positioning and these are encompassed. Some such modified peptides can be expressed from nucleic acids in a cell, others are manufactured synthetically. Similarly, where it is desirable to target the CCR5 interacting agent to one or more specific cell types, this may be achieved either by ex vivo manipulation of target cells, or incorporation of targeting moieties able to bind specifically to target cells or tissues such as ECM binding moieties, as known in the art.

A “conservative amino acid substitution” is one in which the naturally or non-naturally occurring amino acid residue is replaced with a naturally or non-naturally occurring amino acid residue having a similar side chain. Families of amino acid residues having similar side chains have been defined in the art. These families include amino acids with basic side chains (e.g., Lys, Arg, His), acidic side chains (e.g., Asp, Glu), uncharged polar side chains (e.g., Gly, Asn, Gln, Ser, Thr, Tyr, Cys), nonpolar side chains (e.g., Ala, Val, Leu, Ile, Pro, Phe, Met, Trp), beta-branched side chains (e.g., Thr, Val, Ile) and aromatic side chains (e.g., Phe, Trp, His). Thus, a predicted nonessential amino acid residue in a CCR5, for example, may be replaced with another amino acid residue from the same side chain family. Other examples of acceptable substitutions are substitutions based on isosteric considerations (e.g. norleucine for methionine) or other properties (e.g. 2-thienylalanine for phenylalanine). A full amino acid sub-classification is set out in Table 2 and exemplary substitutions are set out in Table 3.

CCR5 interacting peptides may comprise modifications known to modify the pharmacokinetic features of peptides, such as by increasing protease resistance in vivo.

In one embodiment, the peptide comprises one or more of a linker or spacer such as GGS or repeats of GGS and variants known in the art), a modified or non-natural or non-proteogenic amino acid, a modified side-chain, a modified backbone, terminal modified groups or comprises a modified spatial constraint or is a D-retro-inverso peptide. In one embodiment, the peptide is a pseudopeptide, peptoid, azapeptide, cyclized, stapled, ether or lactam peptide or comprises a spatial constraint.

In one embodiment, the CCR5 interacting agent or peptide is conjugated or otherwise attached/bound/expressed with as appropriate to a lipid, carbohydrate, polymer, protein, nanoparticle, peptide, proteoglycan, antibody or fragment or antigen binding form thereof, aptamer, or nucleic acid.

In one embodiment, the CCR5 binding agent specifically binds to muscle cells or muscle cell tissue or associated structures eg, ECM.

In one embodiment, CCR5 interacting agents include physiologically or pharmaceutically acceptable salts, hydrates, sterioisomers, and pro-drugs.

In one embodiment, non-essential amino acids may be altered. Reference to “non-essential” amino acid residue means a residue that can be altered from the wild-type sequence of a polypeptide (e.g., secNAMPT) without abolishing or substantially altering its ability to bind to an endogenous or heterologous CCR5.

In one embodiment, the CCR5 interacting agent comprises or encodes a peptide comprising or consisting or consisting essentially of the amino acid sequence set out in one of SEQ ID NO: 1 to 4 or an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical thereto, optionally together with one or more tissue delivery enhancing or signalling enhancing moieties. In one embodiment, CCR5 interacting agent having a small number of substituted, added or deleted residues, retain the cif motif identified herein (e.g., 40 to about 100 residues within the C-terminal portion of NAMPT that mediate CCR5 binding) retain the ability to interact with and activate satellite cells.

In one embodiment, the CCR5 interacting agent comprises or encodes an amino acid sequence having 1, 2, 3, 4, 5 or 6 conservative or non-conservative amino acid substitution, deletion or addition to the above sequences but retains CCR5 interacting activity.

In one embodiment, the CCR5 interacting agent comprises a nucleic acid molecule from which the CCR5-interacting peptide is expressible.

Examples of suitable polynucleotide sequences are those encoding the peptide/polypeptide sequences set forth in SEQ ID NO: 6 to 9. In certain embodiments, the present disclosure provides polynucleotides comprising nucleic acid sequences comprising a coding sequence for an encodable CCR5 peptide or an encodable CCR5 interacting agent. For example, in some embodiments, nucleic acids of the disclosure comprise, consists essentially of, or consists of a nucleotide sequence that is at least 70%, 75%, 80%, 85%, 86%, 87%, 88%, 89%, 90%, 91%, 92%, 93%, 94%, 95%, 96%, 97%, 98%, 99%, or 100% identical to any one of SEQ ID NO: 6 to 10.

In one embodiment, the nucleic acid molecule is an RNA or DNA or RNA:DNA or a chemically modified form thereof.

In one embodiment, a proportion of at least one type of nucleotide (e.g, cysteine and/or uracil), is chemically modified to increase its stability in vivo.

In one embodiment, the nucleic acid is in the form of a viral or non-viral vector.

In one embodiment, the CCR5 interacting agent is administered to cells ex vivo. The present invention encompasses the use of genetically modified cell depots (e.g. CAR T-cells, TCRs, genetically modified macrophage, etc).

In one embodiment, the CCR5 interacting agent comprises an antibody or antibody fragment that targets the agent specifically to target cells, such as muscle stem cells.

In one embodiment, the present application provides a pharmaceutical or physiological composition comprising a CCR5 interacting agent as defined herein above.

The application enables a method of treating a muscle injury or a person with a diminished or suboptimal ability to repair or regenerate muscle, comprising administering to the subject an effective amount of a composition comprising a CCR5 interacting agent sufficient to stimulate muscle stem cell proliferation and muscle regeneration.

In another aspect, this disclosure is directed to an agent or composition comprising a CCR5-interacting agent as described herein.

Compositions include physiologically or pharmacologically or pharmaceutically acceptable vehicles that are not biologically or otherwise undesirable. Pharmacologically acceptable salts, esters, pro-drugs, or derivatives of a compound described here is a salt, ester, pro-drug, or derivative that is not biologically or otherwise undesirable.

In some embodiments, the agent is modified. Peptide and agent activity are tolerant to additional moieties, flanking residues and substitutions within the defined boundaries. Similarly backbone modifications and replacements, side-chain modifications and N and C-terminal modifications are conventional in the art. Generally, the modification is to enhance stability or pharmacological profile, targeting/delivery.

For example, peptide cyclisation or stapling is conventional for enhancing peptide stability. In another embodiment, peptides or agents are in the form of micro or nano-particles or bubbles, gels, liposomes, conjugates or fusion proteins comprising moieties adapted for stability, delivery or specificity to the target tissue.

In one embodiment, agents or their encoding nucleic acids where appropriate are assembled in liposomes, hydrogels, emulsions, viral vectors, viral-like particles or virosomes.

In one embodiment, specific binding moieties such as antibody or antibody fragments or mimics are used to target agents to the muscle environment.

In one embodiment, peptide agents are delivered through biological synthesis in vivo such as via delivery of mRNA, gene editing such as CRISPR components, or bacteria or cells.

Compositions generally comprise a CCR5-interacting peptide, peptidomimetic or an encoding nucleic acid where appropriate, and a pharmaceutically acceptable carrier and/or diluent. In one embodiment, the carrier may be a nanocarrier.

In one embodiment, the CCR5-interacting agents of the present disclosure are not naturally occurring molecules, but instead are modified forms of naturally occurring molecules which do not possess certain features or functions of the naturally occurring full length molecules. For example, NAMPT enzymatic activity may be absent.

In another embodiment, the CCR5-interacting peptide is constrained by means of a linker which is covalently bound to at least two amino acids in the peptide. Various cyclisation strategies are known in the art to increase stability and cellular permeability.

In some embodiments, the CCR5 interacting agent is delivered in the form of nucleic acid molecules encoding same or pro-drugs thereof or vectors comprising nucleic acid molecules encoding same or pro-drugs thereof. In one embodiment, the nucleic acid is mRNA. The CCR5 interacting agent may bind the surface of the muscle stem cell or function internally to stimulate signalling and proliferation.

In another embodiment, the CCR5 interacting agent is combined, in addition to other pharmaceutically acceptable carriers, with amphipathic agents such as lipids which exist in aggregates as micelles, insoluble monolayers, liquid crystals or lamellar layers in aqueous solution.

In one embodiment, the disclosure enables a composition comprising a CCR5-interacting agent as described herein which interacts with endogenous CCR5 proteins for use as a medicament or for use in therapy.

In another aspect, the present disclosure enables a composition for stimulating muscle stem cell proliferation comprising a CCR5-interacting peptide or a nucleic acid molecule from which the peptide is expressible.

In one embodiment, the subject composition is co-administered with a second physiologically active, therapeutic or prophylactic or regenerative agent. Illustrative cytokines include without limitation one or more of IGF-1, TGF-b, GDF-5, bFGF, PDGF-b3, IL-4.

In another aspect, the present disclosure provides for the use of the CCR5 interacting agent in the manufacture of a medicament for stimulating muscle regeneration or in stem cell therapy.

In one embodiment, the application provides screening assays for CCR5-interacting agents as described herein, comprising assessing the ability of agents to induce muscle stem cell proliferation and muscle generation or indicators thereof.

Peptide-based therapeutics provide useful molecules because they are known to be potent and selective against biological targets that are otherwise difficult to manipulate with small molecules. To improve the pharmacokinetic properties of linear peptides, modified peptides have been successfully developed.

The peptides of the present disclosure comprise amino acids. Reference to “amino acid” includes naturally occurring amino acids or non-naturally occurring amino acids.

Peptide compounds are generally and conventionally modifiable by addition of moieties, flanking peptide residues, and substitutions within understood parameters.

Peptides can furthermore comprise routine modified backbones, side chains, peptide bond replacements, and terminal modifications using standard peptide chemistries.

The amino acids incorporated into the amino acid sequence described herein may be L-amino acids, D-amino acids, L-β-homo amino acids, D-β-homo amino acids or N-methylated amino acids, sugar amino acids, and/or mixtures thereof. Non-natural amino acids may not be recognised by proteases and may therefore alter the half-life. In one embodiment, the D-retro inversion sequence is employed.

Non-naturally occurring amino acids include chemical analogues of a corresponding naturally occurring amino acid. Examples of unnatural amino acids and derivatives include, but are not limited to, 4-amino butyric acid, 6-aminohexanoic acid, 4-amino-3-hydroxy-5-phenylpentanoic acid, 4-amino-3-hydroxy-6-methylheptanoic acid, t-butylglycine, nor leucine, norvaline, phenylglycine, ornithine, sarcosine, 2-thienyl alanine and/or D-isomers of amino acids.

In one embodiment, peptides are modified to enhance their pharmacodynamics properties using art recognised modifications. Peptides may be substituted, such as alanine substituted, or substituted with cross linkable moieties and/or linked. Suitable residues may comprise additional alpha-carbon substitutions selected from hetero-lower alkyl, hetero-methyl, ethyl, propyl and butyl. Peptide bond replacements such as trifluoroethylamines are used to produce more stable and active peptidomimetics.

Accordingly, cyclic or stapled peptides, peptoids, peptomers, and peptidomimetic forms of peptides are encompassed.

Backbone constrained peptidomimetics and cyclic peptides are protected against exopeptidases. Peptides can be cyclised coupling N- to C-terminus after cleavage. This can be achieved by direct coupling or by introduction of specific functional groups that permit defined cyclization by a biorthogonal reaction. Illustrative modifications Cys-Cys disulphide bridges, inclusion of sidechain modifications to include linkers forming macro lactam peptides, thio ether peptides or stapled peptides etc. Click variants are particularly useful for peptide cyclization. Another approach uses 2-amino-d,l-dodecanoic acid (Laa) couples to the N-terminus and by replacing Asn with the lipoamine.

A more defined structure can be obtained by use of a more rigid back bone with heterocycles, N-methylated amine bonds or methylated alpha-carbon atoms.

Among the techniques used for peptide stapling, the two-component double Cu-catalysed azide-alkyne cycloaddition (CuAAC) strategy constrains the peptides in the bioactive conformation and simultaneously improves pharmacokinetic properties. Moreover, this strategy uses unnatural azido amino acids that can be easily synthesised and facilitates the functionalisation of the staple, fluorescent-labelled tags and photo-switchable linkers. The independent functionalisation of the staple can be particularly useful as the complex functionality is added to the staple rather than the N- or C-terminus of the peptide. In addition, this approach only requires one linear peptide to generate a variety of functionalised stapled peptides, facilitating the exploration of various functionalities on the linker and thus properties of the overall peptide.

Azapeptides are peptide analogs in which one or more of the amino residues is replaced by a semicarbazide. This substitution of a nitrogen for the α-carbon center results in conformational restrictions, which bend the peptide about the aza-amino acid residue away from a linear geometry. The resulting azapeptide turn conformations have been observed by x-ray crystallography and spectroscopy, as well as predicted based on computational models. In biologically active peptide analogs, the aza-substitution has led to enhanced activity and selectivity as well as improved properties, such as prolonged duration of action and metabolic stability.

Half-life may also be increased by acylating or amidating ends. Peptoids are produced with N-alklyated oligoglycines side chains. In some embodiments, peptides may be acetylated, acylated (e.g., lipopeptides), formylated, amidated, phosphorylated (on Ser, Thr and/or Tyr), sulphated or glycosylated.

The term “macrocyclization reagent” or “macrocycle-forming reagent” as used herein refers to any reagent which may be used to prepare a peptidomimetic macrocycle by mediating the reaction between two reactive groups. Reactive groups may be, for example, an azide and alkyne, in which case macrocyclization reagents include, without limitation, Cu reagents such as reagents which provide a reactive Cu(I) species, such as CuBr, Cul or CuOTf, as well as Cu(II) salts such as Cu(CO.sub.2CH.sub.3).sub.2, CuSO.sub.4, and CuCl.sub.2 that can be converted in situ to an active Cu(I) reagent by the addition of a reducing agent such as ascorbic acid or sodium ascorbate.

Macrocyclization reagents may additionally include, for example, Ru reagents known in the art such as Cp*RuCl(PPh.sub.3).sub.2, [Cp*RuCl].sub.4 or other Ru reagents which may provide a reactive Ru(II) species. In other cases, the reactive groups are terminal olefins. In such embodiments, the macrocyclization reagents or macrocycle-forming reagents are metathesis catalysts including, but not limited to, stabilized, late transition metal carbine complex catalysts such as Group VIII transition metal carbene catalysts. For example, such catalysts are Ru and Os metal centers having a +2 oxidation state, an electron count of 16 and pentacoordinated. Additional catalysts are disclosed in Grubbs et al., “Ring Closing Metathesis and Related Processes in Organic Synthesis” Acc. Chem. Res. 1995, 28, 446-452, and U.S. Pat. No. 5,811,515. In yet other cases, the reactive groups are thiol groups. In such embodiments, the macrocyclization reagent is, for example, a linker functionalized with two thiol-reactive groups such as halogen groups.

In one embodiment, a peptidomimetic macrocycle exhibits improved biological properties such as increased structural stability, increased affinity for a target, increased resistance to proteolytic degradation when compared to a corresponding non-macrocyclic polypeptide. In another embodiment, a peptidomimetic macrocycle comprises one or more α-helices in aqueous solutions and/or exhibits an increased degree of α-helicity in comparison to a corresponding non-macrocyclic polypeptide.

For example, the sequence of the peptide can be analyzed and azide-containing and alkyne-containing amino acid analogs of the invention can be substituted at the appropriate positions. The appropriate positions are determined by ascertaining which molecular surface(s) of the secondary structure is (are) required for biological activity and, therefore, across which other surface(s) the macrocycle forming linkers of the invention can form a macrocycle without sterically blocking the surface(s) required for biological activity. Such determinations are made using methods such as X-ray crystallography of complexes between the secondary structure and a natural binding partner to visualize residues (and surfaces) critical for activity; by sequential mutagenesis of residues in the secondary structure to functionally identify residues (and surfaces) critical for activity; or by other methods. By such determinations, the appropriate amino acids are substituted with the amino acids analogs and macrocycle-forming linkers of the invention. For example, for a helical secondary structure, one surface of the helix (e.g., a molecular surface extending longitudinally along the axis of the helix and radially 45-135° degree. about the axis of the helix) may be required to make contact with another biomolecule in vivo or in vitro for biological activity. In such a case, a macrocycle-forming linker is designed to link two carbons of the helix while extending longitudinally along the surface of the helix in the portion of that surface not directly required for activity.

The peptidomimetic macrocycle may comprise a helix in aqueous solution. For example, the peptidomimetic macrocycle may exhibit increased helical structure in aqueous solution compared to a corresponding non-macrocyclic polypeptide. In some embodiments, the peptidomimetic macrocycle exhibits increased thermal stability compared to a corresponding non-macrocyclic polypeptide. In other embodiments, the peptidomimetic macrocycle exhibits increased biological activity compared to a corresponding non-macrocyclic polypeptide. In still other embodiments, the peptidomimetic macrocycle exhibits increased resistance to proteolytic degradation compared to a corresponding non-macrocyclic polypeptide. In yet other embodiments, the peptidomimetic macrocycle exhibits increased ability to penetrate living cells compared to a corresponding non-macrocyclic polypeptide.

The term “amino acid analog” refers to a molecule which is structurally similar to a naturally occurring amino acid and which can be substituted for an amino acid in the formation of a peptidomimetic macrocycle. Amino acid analogs include, without limitation, compounds which are structurally identical to an amino acid, as defined herein, except for the inclusion of one or more additional methylene groups between the amino and carboxyl group or for the substitution of the amino or carboxy group by a similarly reactive group (e.g., substitution of the primary amine with a secondary or tertiary amine, or substitution or the carboxy group with an ester).

The peptide may comprise an N-terminal acetyl, formyl, myristoyl, palmitoyl, carboxyl, 2-furosyl and or a C-terminal hydroxyl, amide, ester or thioester group. In one embodiment, the peptide is acetylated at the N-terminus and amidated at the C-terminus. In one embodiment, chelators are introduced for example DOTA, DPTA. Peptides may be modified by, for example pegylation, lipidation, xtenylation, pasylation and other approaches to extend the half-life of the peptide in vivo or in vitro. In one embodiment, pegylation is used to increase peptide solubility and bioavailability. Various forms of peg are known in the art and include HiPeg, branched and forked Peg, releasable Peg, heterobifunctional Peg with end group NHS esters, malaimeide, vinyl sulphone, pyridyl disulphide, amines and carboxylic acids. Examples of therapeutic pegylated peptides include pegfilagrastin (Neulasta) made Amgen.

Linkers or spacers may be amino acids or nucleic acids or other atomic structures known in the art, typically between 2 and 10 amino acids or nucleotides in length. Spacers should be flexible enough to allow correct orientation of CCR5-interacting constructs as described herein, such as those including nanoparticles, antibody fragments, liposomes, cell penetrating and/or intracellular delivery moieties. One form of spacer is the hinge region from IgG suitable for use when the construct comprises an antigen binding moiety for cellular targeting.

Antigen-binding molecules include for example extracellular receptors, antibodies or antibody fragments (including molecules such as an ScFv). Signal peptides may be present at the N-terminal end. Bispecific antibodies capable of selectively binding to two or more epitopes are known in the art and could be used in the present CCR5 interacting agents to bind for example to the muscle environment or other substrate.

In one embodiment, the peptide is conjugated or otherwise associated (covalent or non-covalent attachment) with a delivery agent. In one embodiment the delivery agent delivers the peptide to tissue, a target cell or cell population.

Derivatives of CCR5 interacting agents include biologically active fragments thereof as described herein comprising the structures described or orthologs. Systematic shortening or alanine scanning or modelling around the conserved motif can be routinely conducted to identify minimal peptides with CCR5 agonist effect.

Derivatives also include molecules having a percent amino acid or polynucleotide sequence identity over a window of comparison after optimal alignment. In one embodiment the percentage identity is at least 80%-99% including any number in between 80 and 99.

Suitable assays for the biological activity of peptides or agents are known to the skilled addressee and are described in the specification.

In some embodiment, markers of peptide activity include upregulation of satellite cell signalling (eg, MAPK), stem cell and myoblast proliferation and differentiation.

In one embodiment, the CCR5-interacting agent is modified with a moiety which is not a naturally occurring amino acid residue. The moiety may be selected from the group consisting of a detectable label, a non-naturally occurring amino acid as described herein, a reactive group, a fatty acid, cholesterol, a lipid, a bioactive carbohydrate, a nanoparticle, a small molecule drug, and a polynucleotide. In one particular embodiment, the moiety is a detectable tag label. In one example the detectable label is selected from the group consisting of a fluorophore, a fluorogenic substrate, a luminogenic substrate, and a biotin. Art recognised tags or labels include affinity agents and moieties for detection include fluorescent and luminescent compounds, metals, dyes. Other useful moieties include affinity tags, biotin, lectins, chelators, lanthanides, fluorescent dyes, FRET acceptor/donors.

In one embodiment, the CCR5-interacting agent which may comprise a detectable label, is accompanied in a kit with a modified control version of the agent wherein the conserved residues of the CCR5 agent are substituted with for example alanine. Kits comprising the agents are proposed for sale and may be used for screening purposes or therapeutic purposes.

Peptides of this type may be obtained through the application of recombinant nucleic acid techniques as, for example, described in Sambrook et al. MOLECULAR CLONING. A LABORATORY MANUAL (Cold Spring Harbour Press, 1989), in particular Sections 16 and 17; Ausubel et al CURRENT PROTOCOLS IN MOLECULAR BIOLOGY (John Wiley & Sons, Inc. 1994-1998), in particular Chapters and 16; and Coligan et al. CURRENT PROTOCOLS IN PROTEIN SCIENCE (John Wiley & Sons, Inc. 1995-1997), in particular Chapters 1, 5 and 6.

Alternatively, peptides of this type may be synthesised using conventional liquid or increasingly solid phase synthesis techniques. For example, initial reference may be made to solution synthesis or solid phase synthesis as described, for example, by Atherton and Sheppard in SOLID PHASE PEPTIDE SYNTHESIS: A PRACTICAL APPROACH (IRL Press at Oxford University, Oxford, England, 1989), see particularly Chapter 9, or by Roberge et al. (1995 Science 269: 202).

Azapeptide synthesis was previously hampered by tedious solution-phase synthetic routes for selective hydrazine functionalization. Recently, the submonomer procedure for azapeptide synthesis, has enabled addition of diverse side chains onto a common semicarbazone intermediate, providing a means to construct azapeptide libraries by solution- and solid-phase chemistry. In brief, aza residues are introduced into the peptide chain using the submonomer strategy by semicarbazone incorporation, deprotonation, N-alkylation, and orthogonal deprotection. Amino acylation of the resulting semicarbazide and elongation gives the desired azapeptide. Furthermore, a number of chemical transformations have taken advantage of the orthogonal chemistry of semicarbazone residues (e.g., Michael additions and N-arylations). In addition, oxidation of aza-glycine residues has afforded azopeptides that react in pericyclic reactions (e.g., Diels-Alder and Alder-ene chemistry). The bulk of these transformations of aza-glycine residues have been developed by the Lubell laboratory, which has applied such chemistry in the synthesis of ligands with promising biological activity for treating diseases such as cancer and age-related macular degeneration. Azapeptide analogues of growth hormone-releasing peptide-6 (His-d-Trp-Ala-Trp-d-Phe-Lys-NH2, GHRP-6) have for example been pursued as ligands of the cluster of differentiation 36 receptor (CD36) and show promising activity for the development of treatments for angiogenesis-related diseases, such as age-related macular degeneration, as well as for atherosclerosis. Azapeptides have also been employed to make a series of conformationally constrained second mitochondria-derived activator of caspase (Smac) mimetics that exhibit promising apoptosis-inducing activity in cancer cells. The synthesis of cyclic azapeptide derivatives was used to make an aza scan to study the conformation-activity relationships of the anticancer agent cilengitide, cyclo(RGDf-N(Me)V), and its parent counterpart cyclo(RGDfV), which exhibit potency against human tumor metastasis and tumor-induced angiogenesis. Innovations in the synthesis and application of azapeptides are described in Acc Chem Res. 2017 Jul. 18; 50(7):1541-1556.

Alternatively, peptides can be produced by digestion of an adaptor polypeptide with proteinases such as endoLys-C, endoArg-C, endoGlu-C and staphylococcus V8-protease. The digested fragments can be purified by, for example, high performance liquid chromatographic (HPLC) techniques. Measures that may be taken to optimize pharmacodynamics parameters of peptides and peptide analogs are described by Werle M. et al (2006) Strategies to improve plasma half-life time of peptide and protein drugs amino Acids 30(4):351-367; and Di L (2014) Strategic approaches to optimising peptide ADME properties AAPS J 1-10.

The CCR5 interacting peptide may be stabilised for example via nanoparticles, liposomes, micelles or for example PEG as known in the art. Methods to form liposomes are described in: Prescott, Ed. Methods in Cell Biology, Volume XIV, Academic Press, New York, N.Y. (1976), p. 33 et seq., the contents of which is incorporated herein by reference. Polymer nanoparticles ideally use surfactants that are not toxic or physically adsorbed to the nanoparticle. In one aspect, biodegradable surfmers are used. For example, biodegradable, poly(ethylene glycol) (PEG)ylated N-(2-hydroxypropyl) methacrylamide (HPMA) based surfmers are synthesized and used to stabilize lipophilic NPs. In particular, the NP core is made from a macromonomer comprising a poly(lactic acid) (PLA) chain functionalized with HPMA double bond. The nanoparticle forming polymer chains are then constituted by a uniform poly(HPMA) backbone that is biocompatible and water soluble and hydrolysable PEG and PLA pendants assuring the complete degradability of the polymer. The stability provided by the synthesized surfmers is studied in the cases of both emulsion free radical polymerization and solution free radical polymerization followed by the flash nanoprecipitation of the obtained amphiphilic copolymers.

Other stabilising or heterologous moieties include NMEG, ECM binding, syndecan binding albumin, albumin binding proteins, immunoglobulin Fc domain.

Traditional Fc fusion proteins and antibodies are examples of unguided interaction pairs, whereas a variety of engineered Fc domains have been designed as asymmetric interaction pairs as described by Spiess et al. (2015) Molecular Immunology 67(2A): 95-106. Fc conjugates may comprise an amino acid sequence that is derived from an Fc domain of an IgG (IgG1, IgG2, IgG3, or IgG4), IgA (IgA1 or IgA2), IgE, or IgM immunoglobulin. Such immunoglobulin domains may comprise one or more amino acid modifications (e.g., deletions, additions, and/or substitutions) that promote hetero or homo dimeric or multimeric amyloid formation within the host cell.

In some embodiments, nanoparticles comprising the CCR5-interacting agents can be further modified by the conjugation of tissue type specific binding agents, antibodies or fragments thereof known in the art.

Other suitable binding agents are known in the art and include antigen binding constructs such as affimers, aptamers, or suitable ligands (receptors) or parts thereof.

Antibodies, such as monoclonal antibodies, or derivatives or analogs thereof, include without limitation: Fv fragments; single chain Fv (scFv) fragments; Fab′ fragments; F(ab′)2 fragments; humanized antibodies and antibody fragments: camelized antibodies and antibody fragments, and multivalent versions of the foregoing. Multivalent binding reagents also may be used, as appropriate, including without limitation: monospecific or bispecific antibodies; such as disulfide stabilized Fv fragments, scFv tandems (scFv) fragments, diabodies, tribodies or tetrabodies, which typically are covalently linked or otherwise stabilized (i.e. leucine zipper or helix stabilized) scFv fragments.

The term “antibody fragments”, as used herein, include any portion of an antibody that retains the ability to bind to the epitope recognized by the full length antibody. Examples of antibody fragments include, but are not limited to, Fab, Fab′ and F(ab′)₂, Fd, single-chain Fvs (scFv), disulfide-linked Fvs (dsFv), and fragments comprising either a V_{L} or V_{H} region. Antigen-binding fragments of antibodies can comprise the variable region(s) alone or in combination with a portion of the hinge region, CH1, CH2, CH3, or a combination thereof. Preferably, the antibody fragments contain all six CDRs of the whole antibody, although fragments containing fewer than all six CDRs may also be functional.

“Single-chain FVs” (“scFvs”) are antigen-binding fragments that contain the heavy chain variable region (V_{H}) of an antibody linked to the light chain variable region (V_{L}) of the antibody in a single polypeptide, but lack some or all of the constant domains of the antibody. The linkage between the V_{H} and V_{L} can be achieved through a short, flexible peptide selected to assure that the proper three-dimensional folding of the V_{L} and V_{H} regions occurs to maintain the target molecule binding-specificity of the whole antibody from which the scFv is derived. scFvs lack some or all of the constant domains of antibodies.

Methods of making receptor-specific binding agents generally, particularly based on natural ligands, but including antibodies and their derivatives and analogs and aptamers, are known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of target-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, New Jersey and the pSKAN Phagemid Display System, commercially available from MoBiTec, LLC of Marco Island, Florida. Aptamer technology is described for example and without limitation in U.S. Pat. Nos. 5,270,163; 5,475,096; 5,840,867 and 6,544,776.

Optionally, one or more modified amino acid residues are selected from the group consisting of: a glycosylated amino acid, a PEGylated amino acid, a farnesylated amino acid, an acetylated amino acid, a biotinylated amino acid, and an amino acid conjugated to a lipid moiety, and an amino acid conjugated to an organic derivatizing agent. CCR5 interacting peptides may comprise at least one N-linked sugar, and may include two, three or more N-linked sugars. Peptides may also comprise O-linked sugars. CCR5 interacting peptides or agents may be produced in a variety of cell lines that glycosylate the protein in a manner that is suitable for patient use, including engineered insect or yeast cells, and mammalian cells such as COS cells, CHO cells, HEK cells and NSO cells. In some embodiments the CCR5 peptide is glycosylated and has a glycosylation pattern obtainable from a Chinese hamster ovary cell line. In most embodiments the CCR5 interacting agent is synthesised and component parts added using techniques known in the art.

In some embodiments, the subject CCR5 interacting agents have a half-life of about 0.5, 1, 2, 3, 4, 6, 12, 24, 36, 48, or 72 hours in a mammal (e.g., a mouse or a human). Alternatively, they may exhibit a half-life of about 0.5, 1, 2, 3, 4, 5, 6, 8, 10, 12, 14, 20, 25, or 30 days in a mammal (e.g., a mouse or a human) depending upon conjugate and carrier features and the mode of administration. In some embodiments, peptides are modified to maximise retention in the muscle tissue and to avoid or minimise systemic circulation. Agents may be administered in a range of retention enhancing compositions known in the art, such as gels, foams, glues, hydrogels, patches, and films, and the like.

The size of peptide may be modified to alter its hydrodynamic radium and renal clearance. PEGylation and lipidation often with linkers are established modifications to increase serum half life of agents by reducing clearance and protection from proteases. Second-generation PEGylation processes introduced the use of branched structures as well as alternative chemistries for PEG attachment. In particular, PEGs with cysteine reactive groups such as maleimide or iodoacetamide allow the targeting of the PEGylation to a single residue within a peptide reducing the heterogeneity of the final product. Furthermore, biodegradable hydrophilic amino acid polymers that are functional analogs of PEG have been developed, including XTEN (see US 20190083577) and PAS that are homogeneous and readily produced. Chemical linkage of antibody to peptide as developed by ConX illustrate a range of hybrid peptide half life extension methods that promise to overcome may of the disadvantages of earlier methods.

Oral and injectable solution solubilizing excipients include water-soluble organic solvents (polyethylene glycol 300, polyethylene glycol 400, ethanol, propylene glycol, glycerin, N-methyl-2-pyrrolidone, dimethylacetamide, and dimethylsulfoxide), non-ionic surfactants (Cremophor EL, Cremophor RH 40, Cremophor RH 60, d-α-tocopherol polyethylene glycol 1000 succinate, polysorbate 20, polysorbate 80, Solutol HS 15, sorbitan monooleate, poloxamer 407, Labrafil M-1944CS, Labrafil M-2125CS, Labrasol, Gellucire 44/14, Softigen 767, and mono- and di-fatty acid esters of PEG 300, 400, or 1750), water-insoluble lipids (castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil and palm seed oil), organic liquids/semi-solids (beeswax, d-α-tocopherol, oleic acid, medium-chain mono- and diglycerides), various cyclodextrins (α-cyclodextrin, β-cyclodextrin, hydroxypropyl-β-cyclodextrin, and sulfobutylether-β-cyclodextrin), and phospholipids (hydrogenated soy phosphatidylcholine, distearoylphosphatidylglycerol, 1-α-dimyristoylphosphatidylcholine, 1-α-dimyristoylphosphatidylglycerol). The chemical techniques to solubilize agents for oral and injection administration include pH adjustment, cosolvents, complexation, microemulsions, self-emulsifying drug delivery systems, micelles, liposomes, and emulsions.

### Constructs/Vectors

A construct or vector for expressing a CCR5 binding agent from a recipient cell can comprise one or more DNA regions comprising a promoter operably linked to a nucleotide sequence encoding the peptide. The promoter can be inducible or constitutive. Examples of suitable constitutive promoters include, e.g., an immediate early cytomegalovirus (CMV) promoter, an Elongation Growth Factor-1a (EF-1a) gene promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Examples of inducible promoters include, but are not limited to a metallothionine promoter, a glucocorticoid promoter, a progesterone promoter, and a tetracycline promoter.

The expression constructs may be generated by any suitable method including recombinant or synthetic techniques, utilizing a range of vectors known and available in the art such as plasmids, bacteriophage, baculovirus, mammalian virus, artificial chromosomes, among others. The expression constructs can be circular or linear, and should be suitable for replication and integration into eukaryotes. Viruses, which are useful as vectors include, but are not limited to, retroviruses, adenoviruses, adeno-associated viruses, herpes viruses and lentiviruses. A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to the subject stem cells. A number of retroviral systems are known in the art.

In a specific embodiment of the present invention, where the peptide is provided as a nucleic acid encoding the peptide, the nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide or other intracellular targeting moiety. Alternatively, a nucleic acid can be introduced intracellularly and incorporated within host cell DNA for expression.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” may be used and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura el al. (2000) Nucleic Acids Research 28:292, herein incorporated by reference in its entirety for all purposes. Computer algorithms for codon optimization of a particular sequence for expression in a particular host are also available (see, e.g., Gene Forge).

A nucleic acid molecule as described herein may in any form such as DNA or RNA, including in vitro transcribed RNA or synthetic RNA. Nucleic acids include genomic DNA, cDNA, mRNA, recombinantly produced and chemically synthesized molecules and modified forms thereof. A nucleic acid molecule may be single stranded or double stranded and linear or closed covalently to form a circle. The RNA may be modified by stabilizing sequences, capping, and polyadenylation. RNA or DNA and may be delivered as plasmids to express the peptide. RNA-based approaches are routinely available.

The term “RNA” relates to a molecule which comprises ribonucleotide residues and preferably being entirely or substantially composed of ribonucleotide residues. “Ribonucleotide” relates to a nucleotide with a hydroxyl group at the 2-position of a β-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs or analogs of naturally-occurring RNA.

An optimised mRNA based composition could comprise a 5′ and 3′ non translated region (5′-UTR, 3′-UTR) that optimises translation efficiency and intracellular stability as known in the art. In one embodiment, removal of uncapped 5′-triphosphates can be achieved by treating RNA with a phosphatase. RNA may have modified ribonucleotides in order to increase its stability and/or decrease cytotoxicity. For example, in one embodiment, in the RNA, 5-methylcytidine is substituted partially or completely, for cytidine. In one embodiment, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In one embodiment, this guanosine is methylated at the 7-position. The term “conventional 5′-cap” refers to a naturally occurring RNA 5′-cap, preferably to the 7-methylguanosine cap. The term “5′-cap” includes a 5′-cap analog that resembles the RNA cap structure and is modified to possess the ability to stabilize RNA and/or enhance translation of RNA. Providing an RNA with a 5′-cap or 5′-cap analog may be achieved by in vitro transcription of a DNA template in the presence of said 5′-cap or 5′-cap analog, wherein said 5′-cap is co-transcriptionally incorporated into the generated RNA strand, or the RNA may be generated, for example, by in vitro transcription, and the 5′-cap may be attached to the RNA post-transcriptionally using capping enzymes, for example, capping enzymes of vaccinia virus.

A further modification of RNA may be an extension or truncation of the naturally occurring poly(A) tail or an alteration of the 5′- or 3′-untranslated regions (UTR) such as introduction of a UTR which is not related to the coding region of said RNA, for example, the exchange of the existing 3′-UTR with or the insertion of one or more, preferably two copies of a 3′-UTR derived from a globin gene, such as alpha2-globin, alpha1-globin, beta-globin. RNA having an unmasked poly-A sequence is translated more efficiently than RNA having a masked poly-A sequence. In order to increase stability and/or expression of the RNA it may be modified so as to be present in conjunction with a poly-A sequence, preferably having a length of 10 to 500, more preferably 30 to 300, even more preferably 65 to 200 and especially 100 to 150 adenosine residues. In order to increase expression of the RNA it may be modified within the coding region so as to increase the GC-content to increase mRNA stability and to perform a codon optimization and, thus, enhance translation in cells. Modified mRNA may be synthesised enzymatically and packaged into nanoparticles such as lipid nanoparticles and administered, for example intramuscularly.

The nucleic acid molecule can be entrapped in microcapsules prepared, for example, by coacervation techniques or by interfacial polymerization, in colloidal drug delivery systems (e.g., liposomes, microspheres, microemulsions, nanoparticles and nanocapsules), or in macroemulsions. Such techniques are known in the art and disclosed in Remington, the Science and Practice of Pharmacy, 20th Edition, Remington, J., ed. (2000). Targeted delivery of agents to particular cell subsets can enhance the therapeutic index. Antibody targeted agents that bind to cells comprising an antigen recognized by the antibody or binding fragments thereof. This include for example maleimide functionalized PEG-PLGA polymeric nanoparticles, or simply combining the CCR5 interacting peptide in a composition comprising a delivery moiety or shuttle agent.

Ex vivo approaches contemplate the administration of gene editing such as CRISPR components to modify cells to contain or express a CCR5 interacting agent as described herein.

### Small Molecule Agonists

SMAs are developed using the technology described herein or as known in the art. Small molecules are further selected as capable of activating a CCR5 receptor with an ED50 of less than 1000 nM, less than 500 nM, less than 250 nM, less than 200 nM, less than 100 nM, less than 50 nM, less than 25 nM, less than 20 nM, less than 10 nM, less than 1 nM, less than 0.1 nM, less than 0.01 nM, or less than 0.001 nM. In one embodiment CCR5 interacting agonists are selective for CCR5 or satellite cell CCR5.

### Administration

In accordance with this disclosure, the compositions or agents comprising or encoding CCR5 binding agents disclosed herein can be administered to patients for would healing or to delay, maintain, or regenerate muscle in various conditions associated with muscle loss or diminished ability to regenerate functionally.

The compositions may be delivered by injection, by topical or mucosal application, by inhalation or via oral route including modified release modes, over periods of time and in amounts which are effective to stimulate muscle regeneration levels in a subject. Administration may be topical or systemic (e.g., parenteral via for example intravenous, intraperitoneal, intradermal, sub cutaneous or intramuscular routes) or targeted. In one embodiment, administration of CCR5-interacting agent is systemic or directly to a wound. Sub cutaneous or intramuscular routes may be directly to an affected muscle tissue.

CCR5 interacting agents can be formulated in the form of ointments, creams, patches, powders, or other formulations suitable for topical formulations. Small molecular weight CCR5 agonist formulations can deliver the agent from skin to deeper muscle tissue. Accordingly, such formulations may comprise one or more agents that enhance penetration of active ingredient through skin. For topical applications, the CCR5 agents can be included in wound dressings and/or skin coating compositions.

The amount of the agent to be administered may be determined by standard clinical techniques by those of average skill within the art. In addition, in vitro assays may optionally be employed to help identify optimal dosage ranges. The precise dose to be employed will also depend on the nature of the agent and other clinical factors (such as the condition of the subject their weight, age, other conditions, the route of administration and type of composition (cellular, scaffolded, hydrogel baes or oral formulations). The precise dosage to be therapeutically or prophylactically effective and non-detrimental can be determined by those skilled in the art. Pharmaceutical compositions are conveniently prepared according to conventional pharmaceutical compounding techniques. See, for example, Remington, the Science and Practice of Pharmacy, 20th Edition, Remington, J., ed. (2000) and later editions.

Reference to an effective amount includes a therapeutically or physiologically or regeneratively effective amount. A “therapeutically-effective amount” as used herein means that amount of the composition comprising chemokine receptor agonist activity which is effective for producing some desired therapeutic effect in at least a sub-population of cells in an animal at a reasonable benefit/risk ratio applicable to any medical treatment. For example, an amount of a CCR5 agonist administered to a subject that is sufficient to produce a statistically significant, measurable muscle repair or regeneration. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

As used herein, the term “administer” refers to the placement of a composition into a subject by a method or route which results in at least partial localization of the composition at a desired site such that desired effect is produced. Routes of administration suitable for the instant compositions with vary depending upon its format and include both local and systemic administration. Generally, local administration results in more CCR5 agonist or cell treated with CCR5 agonist activity being delivered to a specific location as compared to the entire body of the subject, whereas, systemic administration results in delivery to essentially the entire body of the subject. One method of local administration is by intramuscular injection.

In accordance with the present invention, the term “administering” also include transplantation of a cell into a subject. As used herein, the term “transplantation” refers to the process of implanting or transferring at least one cell into a subject. The term “transplantation” includes, e.g., autotransplantation (removal and transfer of cell(s) from one location on a patient to the same or another location on the same patient), allotransplantation (transplantation between members of the same species), and xenotransplantation (transplantations between members of different species). Skilled artisan is well aware of methods for implanting or transplantation of stem cells for muscle repair and regeneration, which are amenable to the present invention. See for example, U.S. Pat. No. 7,592,174 and U.S. Pat. Pub. No. 2005/0249731, content of both of which is herein incorporated by reference.

As described herein regeneration of muscle tissue by the present methods is associated with minimal fibrosis. Specifically, the methods and agents described herein reduce and/or inhibit formation of scar-like tissue in the damaged or non-regenerating or atrophying muscle tissue. Accordingly, in some embodiments, formation of scar-like tissue formation in the damaged muscle tissue is reduced by at least 5%, at least 10%, at least 20%, at least 30%, at least 40%, at least 50%, at least 60%, at least 70%, at least 80%, at least 90%, at least 95%, or 100% relative to a control without the present agents. Adipose deposition may be similarly reduced.

However, suitable dosage ranges for intravenous administration of the peptide of the present invention are generally about 1.25-5 micrograms of active compound per kilogram (Kg) body weight. Suitable dosage ranges for intranasal administration are generally about 0.01 μg/kg body weight to 1 mg/kg body weight. Effective doses may be extrapolated from dose-response curves derived from in vitro or animal model test systems. Suppositories generally contain active ingredient in the range of 0.5% to 10% by weight; oral compositions preferably contain 10% to 95% active ingredient.

By “derivative” is meant an agent or active that has been derived from the basic sequence of NAMPT or comprises a cif motif by modification of the amino acid sequence, or, for example by conjugation or complexing or expression (eg, as a fusion protein) with other chemical moieties or by post-translational modification techniques as would be understood in the art. The term “derivative” also includes within its scope alterations that have been made to a parent sequence including additions, or deletions that provide for functionally equivalent or functionally enhanced molecules.

By “isolated” is meant material that is substantially or essentially free from components that normally accompany it in its native state.

The term “subject,” includes patient, and refers to any subject of medical or veterinary interest. Subjects may be a vertebrate subject, such as mammalian subject (e.g, bovines, pigs, dogs, cats, equine, lama, camelids, etc.), non-mammals, reptiles birds, fish. The subject includes a human, for whom prophylaxis or therapy is desired. The subject may be in need of prophylaxis or treatment for a cancer, wound care, sarcopenia or other pathology, disease, disorder or condition associated with tissue degeneration.

The term “polynucleotide” or “nucleic acid” as used herein designates mRNA, RNA, cRNA, cDNA or DNA. The term typically refers to oligonucleotides greater than nucleotides in length.

The term sequence “identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, lie, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” may be understood to mean the “match percentage” calculated by the DNASIS computer program (Version 2.5 for Windows; available from Hitachi Software Engineering Co., Ltd., South San Francisco, California, USA) using standard defaults as used in the reference manual accompanying the software. Amino acid sequence identity may also be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term sequence “similarity” refer to the percentage number of amino acids that are identical or constitute conservative amino acid substitutions as defined in Table 1 below. Similarity may be determined using sequence comparison programs such as GAP (Deveraux et al, 1984 Nucleic Acids Research 12: 387-395). In this way, sequences of a similar or substantially different length to those cited herein might be compared by insertion of gaps into the alignment, such gaps being determined, for example, by the comparison algorithm used by GAP. Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., (2001); and Current Protocols in Molecular Biology, ed. Ausubel el al., Greene Publishing and Wiley-Interscience, New York, (1992) (with periodic updates). Immunology techniques are generally known in the art and are described in detail in methodology treatises such as Current Protocols in Immunology, ed. Coligan et al., Greene Publishing and Wiley-Interscience, New York, (1992) (with periodic updates); Advances in Immunology, volume 93, ed. Frederick W. Alt, Academic Press, Burlington, Mass., (2007); Making and Using Antibodies: A Practical Handbook, eds. Gary C. Howard and Matthew R. Kaser, CRC Press, Boca Raton, Fl, (2006); Medical Immunology, 6th ed., edited by Gabriel Virella, Informa Healthcare Press, London, England, (2007); and Harlow and Lane ANTIBODIES: A Laboratory Manual, Second edition Cold Spring Harbor Laboratory Press, Cold Spring Harbor, N.Y., (2014). Conventional methods of gene transfer and gene therapy may also be adapted for use in the present invention. See, e.g., Gene Therapy: Principles and Applications, ed. T. Blankenstein, Springer Verlag, 1999; Gene Therapy Protocols (Methods in Molecular Medicine), ed. P. D. Robbins, Humana Press, 1997; Viral Vectors for Gene Therapy: Methods and Protocols, ed. Otto-Wilhelm Merten and Mohammed Al-Rubeai, Humana Press, 2011; and Nonviral Vectors for Gene Therapy: Methods and Protocols, ed. Mark A. Findeis, Humana Press, 2010. Amino Acids. 2018 January; 50(1):39-68. doi: 10.1007/s00726-017-2516-0. Epub 2017 Nov. 28. References providing further details of the methods employed herein are described below.
Berger, J., Sztal, T. & Currie, P. D. Quantification of birefringence readily measures the level of muscle damage in zebrafish. Biochemical and biophysical research communications 423, 785-788 (2012).
Seger, C. el al. Analysis of Pax7 expressing myogenic cells in zebrafish muscle development, injury, and models of disease. Developmental Dynamics 240, 2440-2451 (2011).
Ellett, F., Pase, L., Hayman, J. W., Andrianopoulos, A. & Lieschke, G. J. mpeg1 promoter transgenes direct macrophage-lineage expression in zebrafish. Blood 117, e49-e56 (2011).
Scott, E. K. et al. Targeting neural circuitry in zebrafish using GAL4 enhancer trapping. Nature methods 4, 323 (2007).
Pisharath, H., Rhee, J. M., Swanson, M. A., Leach, S. D. & Parsons, M. J. Targeted ablation of beta cells in the embryonic zebrafish pancreas using E. coli nitroreductase. Mechanisms of development 124, 218-229 (2007).
Okuda, K. S. et al. A zebrafish model of inflammatory lymphangiogenesis. Biology open, bio. 013540 (2015).
Evans, R. J. el al. 15-keto-prostaglandin E2 activates host peroxisome proliferator-activated receptor gamma (PPAR-γ) to promote Cryptococcus neoformans growth during infection. PLoS pathogens 15, e1007597 (2019).
Cole, N. J. et al. Development and evolution of the muscles of the pelvic fin. PLoS biology 9, e1001168 (2011).
Morsch, M. el al. In vivo characterization of microglial engulfment of dying neurons in the zebrafish spinal cord. Frontiers in cellular neuroscience 9, 321 (2015).
Higashijima, S.-i., Okamoto, H., Ueno, N., Hotta, Y. & Eguchi, G. High-frequency generation of transgenic zebrafish which reliably express GFP in whole muscles or the whole body by using promoters of zebrafish origin. Developmental biology 192, 289-299 (1997).
Pipalia, T. G. et al. Cellular dynamics of regeneration reveals role of two distinct Pax7 stem cell populations in larval zebrafish muscle repair. Disease Models and Mechanisms 9, 671-684 (2016).
Ando, K., Shibata, E., Hans, S., Brand, M. & Kawakami, A. Osteoblast production by reserved progenitor cells in zebrafish bone regeneration and maintenance. Developmental cell 43, 643-650. e643 (2017).
Westerfield, M. The zebrafish book: a guide for the laboratory use of zebrafish (Danio rerio). (University of Oregon press, 2007).
Schindelin, J. et al. Fiji: an open-source platform for biological-image analysis. Nature methods 9, 676 (2012).
Ollion, J., Cochennec, J., Loll, F., Escude, C. & Boudier, T. TANGO: a generic tool for high-throughput 3D image analysis for studying nuclear organization. Bioinformatics 29, 1840-1841 (2013).
Pisharath, H. & Parsons, M. J. in Zebrafish 133-143 (Springer, 2009).
Nguyen, P. D. et al. Muscle stem cells undergo extensive clonal drift during tissue growth via Meox1-mediated induction of G2 cell-cycle arrest. Cell Stem Cell 21, 107-119. e106 (2017).
Inoue, D. & Wittbrodt, J. One for all—a highly efficient and versatile method for fluorescent immunostaining in fish embryos. PloS one 6, e19713 (2011).
Figeac, N., Serralbo, O., Marcelle, C. & Zammit, P. S. ErbB3 binding protein-1 (Ebp1) controls proliferation and myogenic differentiation of muscle stem cells. Developmental biology 386, 135-151 (2014).
Thisse, C. & Thisse, B. High-resolution in situ hybridization to whole-mount zebrafish embryos. Nature protocols 3, 59 (2008).
de Vrieze, E., Sharif, F., Metz, J. R., Flik, G. & Richardson, M. K. Matrix metalloproteinases in osteoclasts of ontogenetic and regenerating zebrafish scales. Bone 48, 704-712 (2011).
Yaffe, D. & Saxel, O. Serial passaging and differentiation of myogenic cells isolated from dystrophic mouse muscle. Nature 270, 725 (1977).

The present application discloses the following methods useful in the practise of the described subject matter, without limitation.

### Zebrafish Strains and Maintenance

Existing transgenic lines used were, TgBAC(par3a:GFP)ⁱ¹⁵⁰ (referred to as TgBAC(par3a:GFP))⁴⁰, Tg(mpeg1:mCherry)^{gl23} (referred to as Tg(mpeg1:mCherry))⁴¹, Tg(mpeg1:GAL4FF)^{gl25} (referred to as Tg(mpeg1:GAL4FF))⁴¹, Tg(UAS-E1b:Kaede)^{s1999t} (referred to as Tg(UAS:Kaede))⁴², Tg(UAS-E1b:Eco.NfsB-mCherry)^{c264} (referred to as Tg(UAS:NfsB-mcherry)⁴³, Tg(−8mpx:KALTA4)^{gl28} (referred to as Tg(mpx:KALTA4))^{44,45}, Tg(actc1b:EBFP2)^{pc5} (referred to as Tg(actc1:BFP))⁴⁶, Tg(ubi:secAnnexinV-mVenus)^{mq8Tg} (referred to as Tg(ubi:secAnnexinV-mVenus))⁴⁷, Tg(actc1b:GFP)^{zf10} (referred to as Tg(actc1b:GFP))⁴⁸. All experiments were conducted in accordance with Monash University guidelines and approved by the local ethics committee. All procedures involving animals at the Hubrecht Institute were approved by the local animal experiments committees and performed in compliance with animal welfare laws, guidelines and policies, according to national and European law. Staging and husbandry were performed as previously described (Westerfield, M. The zebrafish book: a guide for the laboratory use of zebrafish (Danio rerio). (University of Oregon press, 2007)).⁵¹. All embryos were maintained in Ringer's solution at 28.5° C. and treated with 0.003% 1-phenyl-2-thiourea (PTU) (Sigma-Aldrich) from 8 hpf.

### Generation and Genotyping Zebrafish Mutant Lines

Mutations in nampta and ccr5 were generated using the CRISPR/Cas9 system. Synthetic guide RNAs (gRNAs) targeting genes of interest were generated as crRNA:tracrRNA duplexes (Alt-R® CRISPR-Cas9 system, IDT). Gene-specific crRNA sequences were selected using the Alt-R® CRISPR-Cas9 custom guide RNA design tool (IDT) (nampta crRNA: 5′-acgacaagacggtcttctatGGG-3′, ccr5 crRNA_1: 5′-gtagcacccccatgcaacaaTGG-3′, ccr5 crRNA_2: 5′-attttcctgataatacatccTGG-3′). Gene-specific crRNAs were heteroduplexed to universal tracrRNA according to manufacturer recommendations to generate bipartite gRNAs. Mutations were generated by injecting gRNA (nampta mutant generated using single gRNA, ccr5 mutant generated using dual gRNAs (ccr5 crRNA_1+cr5 crRNA_2)) and recombinant Cas9 protein (Alt-R® S.p Cas9 Nuclease, IDT) into the blastomere of one-cell stage wild-type embryos. Injected embryos were grown to adulthood, outcrossed into wild-type zebrafish and screened to identify founders containing germline mutations. Identified mutants of interest; nampta c.180_182delinsTCCGTCTTGCTGACCTTTCCCCAGCAG (p.Try61Profs*4) (referred to as nampta^{pc41}) and ccr5 c.66_578delins ACCCCTATGCAACATCATTTTTACCAATGAGCAAATGGATTTAAACAAGAG AAAATCCTGCCAACTTGATTTTCCTGATAATACATAATA (p.Pro24Leufs*28) (referred to as ccr5^{pc42}). For genotyping nampta mutants, DNA was isolated from clipped fins (adults) or whole embryos and used in a PCR reaction with the oligonucleotides Nampta_F (5′-tgccgtgagaagaagacaga-3′) and Nampta_R (5′-gcaatcaattgccttacctttt-3′) (PCR product size, nampta 117 bp and nampta⁴¹ 141 bp). For genotyping ccr5 mutant, a PCR was performed with the oligonucleotides Ccr5_F (5′-aacgaaactgggcatgtagc-3′) and Ccr5_R (5′-ccgggaataacaaaagctca-3′) (PCR product size, ccr5 618 bp and ccr5^{pc42} 173 bp).

### Larval Zebrafish Muscle Injury

4dpf larvae were anaesthetized in 0.01% tricaine (MS-222) (Sigma-Aldrich) in Ringer's solution. Mechanical injures were targeted either to the dorsal or ventral myotome above the cloaca when the larvae is oriented dorsal to the top, anterior to the left. Needle-stick injury was carried out as previously described (Gurevich, D. B. el al. Asymmetric division of clonal muscle stem cells co-ordinate muscle regeneration in vivo. Science 353 6295 aad9969 (2016)). Briefly, the myotome was subjected to a single 30-gauge needle puncture that generates an extensive injury with many damaged muscle fibres. For laser-induced injury, anaesthetised larvae were mounted in a thin layer of 1% low-melt agarose in Ringer's solution. Injuries were carried out using a UV-nitrogen laser pulsed through a coumarin 440 nm dye cell coupled to a Zeiss Axioplan microscope (MicroPoint Laser System, Andor Technology). On average a laser injury required pulses for 5-10 sec from laser beams focused through a 40× water immersion objective. For time-lapse analysis of the immediate response to injury, muscle fibre ablations were achieved using a SIM scanner (Olympus) at 790 nm and 200 msec dwell time at 100% laser power on an Olympus FVMPE-RS upright multi-photon microscope equipped with a 25×/1.05 water immersion objective. Injury-responding macrophages were tracked using the manual tracking plugin in Fiji.

### Mouse Volumetric Muscle Loss Injury and Repair Assessment

Injury: male C57BL/6J mice aged between 10-12 weeks were anesthetised and shaved on the hind left leg. A unilateral incision measuring approximately 1 cm was made exposing the underlying fascia. The left hind limb was extended and exteriorised via the incision site by retracting the surrounding tissue. A 3×4 mm full thickness segment of the rectus femoris muscle was removed. Directly after, the injury site was filled with fibrin hydrogel with or without 200 ng or 500 ng of hrNAMPT₍₁₎ (hydrogel components; 40 μl, 8 mg/ml human fibrinogen (FIB3, Enzyme Research Laboratories), 4 U/ml bovine thrombin (T4648, Sigma), 5 mM CaCl₂, 17 μg/ml of aprotinin (ab146286, Abcam)) which polymerized in the defect. Then, the soft tissue was closed with stitches.

Histology: 10 days after treatment, animals were sacrificed and the wounds were harvested for histological analysis. The defect site and associated proximal and distal segment of the quadriceps muscle (including the rectus femoris, vastus medialis and vastus lateralis) were excised and embedded. Histological analysis was performed on serial paraffin sections (4 μm sections collected passing the central portion of the wound). Multiple sections were stained with Masson's Trichrome (to detect collagen deposition) and the extent of fibrosis (represented by a blue stain) was measured by histomorphometric analysis using ImageJ software (version 1.51h, National Institutes of Health, USA). To maintain uniformity between samples, the length of the vastus medialis taken at multiple depths ranging from 1.0 mm-3.0 mm serves as a reference between tissue sections to determine the depth of sectioning. For fibrotic quantification, average muscle fibrosis area at each depth was scored and normalised with the area of the rectus femoris. Total area of muscle is determined by calculating the average area of rectus femoris at each depth.

Immune cell profiling and PAX7⁺ cell quantification with flow cytometry: 4, 6 or 8 days after treatment with either 0.5 μg of hrNAMPT₍₁₎ delivered by fibrin hydrogel or control fibrin hydrogel only, mice were euthanised via CO₂ asphyxiation. The defect site and associated proximal and distal segment of the quadricep muscles were isolated and placed into 890 μl of complete RPMI (with 10% FBS and 2 mM Glutamax, Life Technologies). The tissue was minced with surgical scissors and 100 μl of 10 mg/ml Collagenase II (Sigma-Aldrich) and 10 μl of 10 mg/ml DNAse I (Biolabs), while 100 μl of dispase II (10 mg/ml) was added into the digestion for PAX7 acquisition. The mixture was vortexed and incubated at 37° C. for 45 min. The collagenase was then inactivated with 500 μl ice-cold PBS, 5% FBS, 5 mM EDTA. The mixture was strained subsequently through 70 μm and 40 μm filters. The cell suspension was further diluted with 1 ml complete RPMI and centrifuged for 10 min at 300× g. The supernatant was discarded and the pellet was resuspended in 250 μl complete RPMI and aliquoted into wells of a 96-well U bottom plate for antibody staining. The cell solutions were centrifuged, supernatant discarded, and washed with PBS. The cell viability stain used was 100 μl of Zombie Aqua (Biolegend) Live-Dead dye diluted in PBS (1:400 dilution) and incubated for 30 min at 4° C. The cells were then blocked with FcX (anti-CD16/32 antibodies, Biolegend, 1 μg/ml) flow cytometry buffer (PBS, 5% FBS). The cells were kept for 20 min at 4° C., washed with flow cytometry buffer and centrifuged. Primary surface antibody staining was done in 2 separate stains with 100 μl of anti-mouse antibody cocktail (Biolegend) diluted in flow cytometry buffer: T cell stain with 2 μg/ml of anti-CD4 (clone RM4.5, #100516), anti-CD8 (clone 53-6.7, #100738), and anti-CD3 (clone 17A2, #100220. Neutrophil and macrophage stain with 2 μg/ml of anti-CD11b (clone M1/70, #101208), 1 μg/ml anti-Ly6G (clone 1A8, #127628), 4 μg/ml anti-F4/80 (clone BM8, #123147), 10 μg/ml anti-CD80 (clone 16-10A1, #104714), and 2.6 μg/ml anti-CD206 (clone C068C2, #141720). Cells were stained for 30 min on ice and washed as described above. For internal Foxp3 staining in the T cell panel, cells were fixed with 100 μl fixation/permeabilisation solution (42080, Biolegend) for 35 min. Then cells were washed and resuspended in 100 μl of flow cytometry buffer with 0.5% Saponin and 5 μg/ml anti-Foxp3 (clone 3G3, #35-5773-U100) for 45 min. Cells were then resuspended in flow cytometry buffer (100 μl) and acquired on the Fortessa x20 (Beckman Coulter). Satellite cell flow cytometry staining was performed with 200 μl of antibody cocktail (Biolegend) diluted in flow cytometry buffer: 5 μg/ml of anti-VCAM/CD106 biotin (clone 429 (MVCAM.A), #105703), 2.5 μg/ml of anti-streptavidin (#405250), 2 μg/ml of anti-CD45 (clone 30-F11, #103114), anti-CD11b (clone M1/70, #101208), anti-Ly6G (clone 1A8, #127607), 1 μg/ml anti-CD31 (clone MEC13.3, #102507). Cells were stained for 45 min on ice and washed as described above. Cells were also stained with 200 μl flow cytometry buffer with 0.5% saponin with intracellular antibody cocktail: Biolegend 1 μg/ml anti-Ki67 (clone 16A8, #652411), NovusBiologicals 10 μg/ml Anti-Pax7 (clone Pax7/497, #NBP2-34706AF488) for 1 h on ice. Cells were then resuspended in flow cytometry buffer (275 μl) with 25 μl of Invitrogen Count Bright Absolute Counting Beads (25,000 beads, #C36950) and acquired on the Fortessa x20 (Beckman Coulter). All events were acquired and the number of PAX7⁺ cell per 10,000 wound cells was calculated using the following formula: PAX7⁺ number in injury=10,000×PAX7⁺ cell count/[(1/25,000)×beads count×(live cell percentage/100)×total cell number count after digestion]. The same calculation was done to quantify the number of proliferative PAX7⁺ cells, utilizing cell count of double positive for PAX7 and Ki67.

Immunofluorescence for frozen sections: immunostaining was performed on 10 μm cryosections using standard protocol with antigen retrieval (10 mM Sodium Citrate, 0.05% Tween 20, pH 6.0). Sections were blocked with 2% BSA, 5% Normal Goat Serum in PBS with 0.3% Triton-X and AffiniPure Fab Fragment Goat Anti-Mouse IgG (H+L) (Jackson Immuno Research Laboratories) to minimise the unspecific binding of a mouse antibody on mouse tissue. Antibodies: mouse anti-mouse Pax7 (2 μg/ml, Developmental Studies Hybridoma Bank) and secondary Alexa Fluor-coupled antibodies (Thermo Fisher). Muscle sarcolemma were visualised by Rhodamine-labelled wheat germ agglutinin (WGA) (Vector Laboratories) and nuclei were visualised by staining with DAPI (Sigma-Aldrich).

Quantification of centrally nucleated muscle fibres: Haematoxylin and Eosin (H&E) staining was performed on 4 μm paraffin embedded sections. The number of nuclear centralisations within a muscle fibre was counted from five serial sections per sample by histomorphometric analysis using ImageJ software (version 1.51h, National Institutes of Health, USA). To maintain uniformity between samples, the length of the vastus medialis taken at multiple depths ranging from 1 to 3 mm serves as a reference between tissue sections to determine the depth of sectioning. For average number of centrally nucleated cell quantification, total nuclear count at each depth was normalised with the area of the rectus femoris.

### Microscopy and Image Analysis

Whole larva time-lapse imaging to track injury-responding macrophages were performed using a Zeiss Lightsheet Z.1 microscope with a 5×/0.16 air objective and environmental controls (28.5° C.). The XY resolution was 1.14 μm, the Z resolution was 5.5 μm, with a lightsheet thickness of 11.68 μm. Total imaging time per larvae was 25 h (1000 3D stacks acquired at 1.5 min intervals), and viability of larvae was confirmed at the end of the imaging session by assessing heart rate. For tracking, macrophage images were first filtered with 3D median filter and then segmented with hysteresis thresholding using algorithms from the 3D ImageJ Suite⁵³. Low and high threshold for hysteresis were chosen visually. The datasets were then time reversed, in order to track cells exiting the injury site. The margins of the wound were manually labelled within ImageJ. The tracking procedure was based on overlapping segmentation of cells between consecutive frames. Cases where two cells were too close and formed one object were designated “merging” as the segmentation algorithm was unable to separate them. Tracked macrophage images were reversed for the visualisation of results.

Line-scanning confocal microscopy for long-term time-lapse imaging and single Z-stack acquisition was performed using a Zeiss LSM 710 upright confocal equipped with a 20×/1.0 water immersion objective. Photoconversions were carried out using the bleaching tool with a 405 nm diode laser.

Time-lapse imaging at high temporal and spatial resolution was performed on an inverted LSM 880 fast AiryScan confocal equipped with a 40×/1.3 oil immersion objective and piezo Z-stage. The voxel size was kept constant at 0.2×0.2×1 μm and depending on the field of view frame rates of 3-18 frames per second were achieved. Photobleaching was assayed post imaging and determined to be minimal for imaging durations of up to 1h.

Fixed and Immunostained cell culture samples were imaged on a Leica DMi8 inverted widefield microscope with a 10× objective. Birefringence imaging was carried out as previously described (Berger, J., Sztal, T. & Currie, P. D. Quantification of birefringence readily measures the level of muscle damage in zebrafish. Biochemical and biophysical research communications 423, 785-788 (2012)), using a Leica DM IRB upright microscope integrated with the Abrio software (CRI Hinds instruments) using a 5× objective. Images were analysed using the software Fiji⁵², whereby the mean grey value of the injury site birefringence is normalised to the region before and after it to calculate relative birefringence of the wound site.

Microscopy images were processed in Adobe Creative Cloud 2018, Fiji⁵² and Imaris 9.2 (Bitplane). Counting macrophage numbers and further 3D analyses were performed by surface rendering their volumes using Imaris. Sphericity analysis assessed a cells shape deviation from a perfect sphere, which is assigned an arbitrary value of one. Sphericity values were generated as a summary statistic of the surface render. Proliferating stem cell counts were carried out on Fiji. The PAX7 and EdU acquisition channels were segmented using the threshold command. The image calculator function was used to generate a masked channel of only EDU positive PAX7 cells. The number of cells was counted using the Analyse Particles command.

### Chemical Treatments

Cell ablation was carried out as previously described (Pisharath, H. & Parsons, M. J. in Zebrafish 133-143 (Springer, 2009)) with minor modifications. Larvae at the appropriate stage were incubated in 5-10 mM metronidazole (Mtz) (Sigma-Aldrich) in Ringer's solution and daily refreshed until experimental end point. Drug treatments were carried out by incubating 4 dpf needle stick injured larvae in 5 and 10 μM cenicriviroc (CVC) (Med Chem Express) and 5 and 10 μM maraviroc (MVC) (Med Chem Express) in Ringer's solution immediately post injury and refreshed daily.

The effect of drug treatment on laser ablation muscle injury was assayed by treating larvae with 5 μM CVC for 2 h prior to injury and with larvae being maintained in the drug until experimental end point. For the NAMPT enzymatic inhibiting experiments, needle stab injured larvae at 5 dpf/1.75 dpi were transferred into Ringer's solution containing 10 μM GMX1778 (Sigma-Aldrich) and maintained as such until experimental end point. Chemokine supplementation experiments were carried out in plasticware coated with 0.1% bovine serum albumin (BSA) overnight (rinsed in Ringer's solution prior to use) to minimise protein adsorption. Larvae were treated with 57 nM recombinant human visfatin (hrNAMPT₍₁₎) (PeproTech), 57 nM recombinant murine CCL8/MCP-2 (mrCCL8) (PeproTech) and combinations with Mtz and CVC.

Cell culture drug supplementation was carried out by adding the following to the growth media of C2C12 cells for 6 h: 1.9, 9.5 and 19 nM hrNAMPT₍₁₎ (PeproTech), 1.9 and 9.5 nM recombinant human visfatin (hrNAMPT₍₂₎)(Enzo Life Sciences), 100 nM CVC (Med Chem Express), 100 nM MVC (Med Chem Express), 100 nM PF-4136309 (PF4) (Med Chem Express), 9.5 nM mrCCL8, 9.5 nM recombinant murine CCL4/MIP-1β (mrCCL4) (PeproTech), 9.5 nM recombinant murine CCL2/MCP-1 ((PeproTech), 500 nM GMX1778 (Sigma-Aldrich) and combinations with hrNAMPT₍₁₎. The following were added to the proliferation media of primary mouse myoblast co-cultures for 24 h; 9.5 nM hrNAMPT₍₂₎ (Enzo Life Sciences), 100 nM CVC (Med Chem Express), 100 nM MVC (Med Chem Express), 100 nM PF4 (Med Chem Express) and 9.5 nM mrCCL4 (PeproTech).

LysoTracker assay: Larvae were incubated in 10 μM LysoTracker™ Deep Red (Thermo Fisher) in Ringer's solution for 1 h in the dark and rinsed 5 times with fresh Ringer's before imaging.

5′-ethnyl-2′-deoxyuridine (EdU) Labelling

Larvae: Labelling and detection was carried out as previously described (Nguyen, P. D. et al. Muscle stem cells undergo extensive clonal drift during tissue growth via Meox1-mediated induction of G2 cell-cycle arrest. Cell Stem Cell 21, 107-119. e106 (2017))⁵⁵ with minor alteration. 6 dpf/2 dpi larvae were pulsed with 2.5 nM EdU (Thermo Fisher) for 1h and chased for a further 1.5 h prior to fixation. Cell culture: Cells were incubated for 1 h in media supplemented with 10 μM EdU. Following Edu pulse, C2C12 cells were fixed immediately, while primary mouse myoblast co-cultures were rinsed in PBS, replaced with media and incubated for a further 2 h following which the cells were fixed. Cells were processed using the Click-iT™ EdU Alexa Fluro™ 647 imaging Kit (Thermo Fisher) following the manufacturer's protocol.

### Immunohistochemistry and In Situ Hybridisation

Antibody staining on whole-mount larvae was carried out as previously described (Inoue, D. & Wittbrodt, J. One for all—a highly efficient and versatile method for fluorescent immunostaining in fish embryos. PoS one 6, e19713 (2011)) and on cultured myoblasts as previously described (Figeac, N., Serralbo, O., Marcelle, C. & Zammit, P. S. ErbB3 binding protein-1 (Ebp1) controls proliferation and myogenic differentiation of muscle stem cells. Developmental biology 386, 135-151 (2014)). Following in situ hybridisation, antibody staining was carried out using standard procedures. Antibodies: mouse anti-Pax7 (1:10, DSHB), chicken anti-GFP antibody (1:500, Thermo Fisher), mouse anti-mCherry antibody (1:500, Abcam), rat anti-mCherry antibody (1:500, Kerafast) (immunohistochemistry), rabbit anti-PBEF1 (anti-NAMPT) antibody (1:50, Sigma-Aldrich) and secondary Alexa Fluro-coupled antibodies (Thermo Fisher). Nuclei were visualised by staining with DAPI (Sigma-Aldrich). In situ hybridisation and probe generation was performed as previously described (Thisse, C. & Thisse, B. High-resolution in situ hybridization to whole-mount zebrafish embryos. Nature protocols 3, 59 (2008)). Antisense probe used: mmp9 (de Vrieze, E., Sharif, F., Metz, J. R., Flik, G. & Richardson, M. K. Matrix metalloproteinases in osteoclasts of ontogenetic and regenerating zebrafish scales. Bone 48, 704-712 (2011). nampt (ENSDARG00000030598) PCR probe containing a T7 RNA polymerase promoter at the 3′ for the antisense probe and an SP6 RNA polymerase promoter at the 5′ for the sense probe was generated using primers 5′-GAGtatttaggtgacactatagGGTTTCATCGCAAGAGACGG-3′ and 5′-GAGtaatacgactcactatagggGCGGAAGCACCTTATAGCCT-3′. Haematoxylin and Eosin staining was performed on 10 μm cryostat cross-sections of 5 and 7 dpf larvae according to standard methods.

### NAMPT Binding to CCR5 (E LISA)

hrNAMPT binding to hrCCR5: ELISA plates (Medium binding, Greiner Bio-One) were coated with 1% BSA or 20 nM of GST-fused recombinant human CCR5 (hrCCR5, Abcam) in PBS overnight at 4° C. Wells were blocked for 1 h at room temperature with 1% BSA in PBS containing 0.05% Tween-20 (PBS-T). Wells were washed 3 times with PBS-T and further incubated with hrNAMPT₍₁₎ (Peprotech) at increasing concentration (0 nM to 800 nM) for 1 h in PBS-T with 0.1% BSA. Bound NAMPT molecules were detected using a biotinylated antibody for NAMPT and HRP-streptavidin (Human PBEF/Visfatin DuoSet ELISA, R&D Systems). Signals obtained on BSA-coated wells were used to remove non-specific binding for each NAMPT concentrations to obtain specific binding values. Specific binding data were fitted by non-linear regression with Prism 7 to obtain the dissociation constant (K_{D}) using A₄₅₀ nm=Bmax*[NAMPT]/(K_{D}+[NAMPT]).

hrNAMPT competitive binding to mrCCR5: ELISA plates (Medium binding, Greiner Bio-One) were coated with 1% BSA or 20 nM of recombinant mouse CCR5 (MyBioSource) in PBS overnight at 4° C. Then, wells were blocked for 1 h at room temperature with 1% BSA in PBS containing 0.05% Tween-20 (PBS-T). Wells were washed 3 times with PBS-T and further incubated with murine CCL4 (Peprotech) at increasing concentration (0 nM to 400 nM) for 1 h in PBS-T with 0.1% BSA containing 100 nM hrNAMPT₍₁₎ (Peprotech). Bound hrNAMPT₍₁₎ molecules were detected using a biotinylated antibody for NAMPT and HRP-streptavidin (Human PBEF/Visfatin DuoSet ELISA, R&D Systems). Signals obtained on BSA-coated wells were used to remove non-specific binding for each hrNAMPT concentrations to obtain specific binding values. Specific binding data were fitted by non-linear regression with Prism 7 to obtain the half maximal inhibitory concentration (IC50) of CCL4 using A₄₅₀ nm=A₄₅₀ nmMin+(A₄₅₀ nmMax−A₄₅₀ nmMin)/(1+10\hat{ }(X−Log IC50)).

NAMPT in macrophage supernatants: Maf/DKO cells and the mouse macrophage cell line Raw 264.7 (ATCC) were cultured in growth media (DMEM+10% FBS+10 ng/ml M-CSF) for 16 h. Protein in collected supernatant was concentrated using Amicon® Ultra-15 centrifugal filter with a 10 kDa nominal molecular weight limit (Merck). NAMPT in supernatant was quantified using Human PBEF/Visfatin DuoSet ELISA (R&D Systems), according to manufacturer's instructions. Cell surface CCR5 receptor concentration: The mouse muscle cell line C2C12 was cultured as described above. Cells were dislodged at 70-80% confluence using a cell scraper and membrane proteins were isolated using an extraction kit (Plasma Membrane Protein Extraction Kit, Abcam). CCR5 concentration in the membrane extract was then measure by ELISA (Mouse Ccr5 ELISA Kit, Biorbyt) and the amount of CCR5 per cell was then calculated using [CCR5]_{(molecules/cell)}=[CCR5]_{(ng/cell)}/CCR5_{mw}*10\hat{ }−9*N₀, where N₀=Avogadro constant.

### Mouse Cytokine Array

Supernatant was collected from Maf/DKO cells cultured for 16 h in growth media (DMEM+10% FBS+10 ng/ml M-CSF). Cytokine array (Proteome profiler array, R&D systems, ARY006) was performed according to manufacturer's instructions. Briefly, nitrocellulose membranes were blocked for 1 h at room temperature on a rocking platform shaker. During the blocking step, 0.7 ml of sample were topped up to 1.5 ml with Array Buffer and incubated with Mouse Cytokine Array Panel A Antibody Cocktail for 1 h at room temperature. Samples were then incubated on membranes overnight at 4° C. on a rocking platform shaker. Membranes were washed prior to incubation with Streptavidin-HRP for 30 min at room temperature. Finally, membranes were developed by adding Chemi Reagent Mix, then imaged on a Biorad Chemidoc MP system and analysed using (Image Lab software, Bio Rad).

### Cell Culture

The mouse muscle cell line C2C12 (Yaffe, D. & Saxel, O. Serial passaging and differentiation of myogenic cells isolated from dystrophic mouse muscle. Nature 270, 725 (1977) were cultured in growth media (Dulbecco's Modified Eagle Medium (4.5 g/l D-Glucose, No L Glutamine, No Sodium Pyruvate (Gibco))+20% Fetal Bovine Solution-One Shot (Gibco)+1% Glut Max 100x (Gibco)). Cells were maintained at 37° C., 5% CO₂. Cells at 70% confluence, passage 8 were extracted from T75 flasks with 0.025% Tryspin EDTA (Gibco), neutralised in growth media, spun at 180×g for 5 min to pellet cells. The cells were then resuspended in 10 ml of fresh growth media. 500 μl of cells were plated on a 8-well on cover glass II (Sarstedt) chamber slide at a density of 1×10³ cells/ml. Cells were left 4 h at 37° C. to re-attach. For drug treatments, the media were supplemented with appropriate doses and cultured for 6 h.

For isolation of primary mouse myoblasts, limb skeletal muscle from E17.5 C57/BL6J mice were minced and digested in 0.125% Trypsin at 37° C. for 20 min. Fibroblasts were depleted by plating cells in 10 cm² tissue culture dishes (2 embryos per dish) in proliferation media (DMEM+20% FBS) for 1 h. Media with non-attached cells was re-plated in gelatin-coated 10 cm² tissue culture dishes in proliferation media for 24 h. Myoblasts were again depleted for fibroblasts prior to co-culturing on gelatin-coated 48 well plates in DMEM+20% FBS+10% L929-conditioned medium. 100,000 myoblasts were plated with either 7,500 MafB/c-Maf deficient (Maf-DKO) macrophages (Aziz, A., Soucie, E., Sarrazin, S. & Sieweke, M. H. MafB/c-Maf deficiency enables self-renewal of differentiated functional macrophages. Science 326, 867-871 (2009)) or 1,000 3T3 cells per well. For drug treatments, the media were supplemented with appropriate doses and cultured for 24 h.

### Cell Surface CCR5 Receptor Concentration

The mouse muscle cell line C2C12 were cultured as described earlier (see above). The mouse macrophage cell line Raw 264.7 (ATCC) were cultured in growth media (Dulbecco's Modified Eagle Medium+10/6 FBS). Cells were dislodged at 70-80% confluence using a cell scraper and membrane proteins were isolated using an extraction kit (Plasma Membrane Protein Extraction Kit, abcam). CCR5 concentration in the membrane extract was then measure by ELISA (Mouse Ccr5 ELISA Kit, Biorbyt) and the amount of CCR5 per cell was then calculated using [CCR5]_{(molecules/cell)}=[CCR5]_{(ng/cell)}/CCR5_{mw}*10\ddot{ }−9*N₀, where N₀=Avogadro constant.

### Fluorescence Activated Cell Sorting (FACS), Single-Cell RNA-Sequencing and Analyses

Injury-responding macrophages were isolated by FACS as previously described (Ratnayake, D. & Currie, P. D. in Myogenesis 245-254 (Springer, 2019)), with the following modifications. 4dpf Tg(mpeg1:mCherry) larvae were subject to needle-stick injury and the injured region dissected at 1, 2 and 3 dpi and tissue dissociated into a single cell suspension. Uninjured larval trunk tissue was also included in the analysis. Cells were sorted using a FACS Aria II (BD biosciences). Live individual macrophages (based on mCherry fluorescence, DAPI exclusion and forward and side scatter properties) were sorted into pre-prepared 384 well plates containing 100-200 nl of CEL-seq primers, dNTPs and synthetic mRNA Spike-Ins contained in 5 μl of Vapor-Lock (Qiagen). Immediately following sorting, plates were spun down and frozen at −80° C. until sequencing.

Single-cell RNA-sequencing libraries were prepared using the SORT-seq platform as previously described (Muraro, M. J. et al. A single-cell transcriptome atlas of the human pancreas. Cell systems 3, 385-394. e383 (2016). In this platform the Cel-Seq2 protocol (Hashimshony, T. et al. CEL-Seq2: sensitive highly-multiplexed single-cell RNA-Seq. Genome biology 17, 77 (2016)) is followed with the aid of robotic liquid handlers. This protocol results in each cell being barcoded, and generating single-cell transcriptomes of all isolated macrophages. Each time point is independently replicated and results in approximately 768 macrophages per time point (3072 macrophages in total) to be individually sequence. Next generation sequencing was carried out using an Illumina NextSeq platform. Paired reads were mapped against the zebrafish reference assembly version 10 (GRC10). FASTQ files were processed as previously described (Muraro, M. J. et al. (2016); Grin, D. et al. De novo prediction of stem cell identity using single-cell transcriptome data. Cell Stem Cell 19, 266-277 (2016).). Paired end read were aligned to the zebrafish transcriptome using bwa (Li, H. & Durbin, R. Fast and accurate long-read alignment with Burrows-Wheeler transform. Bioinformatics 26, 589-595 (2010)) with a transcriptome dataset with improved 3′ UTR annotations to increase the mapability of transcripts (Junker, J. P. et al. Genome-wide RNA tomography in the zebrafish embryo. Cell 159, 662-675 (2014)). Read 1 was used for assigning reads to correct cells and libraries while read 2 was mapped to gene models. Read counts were first corrected for UMI barcode by removing duplicate reads that had an identical combination of library, cellular and molecular barcodes that were mapped to the same gene. Transcript counts were then adjusted to the expected number of molecules based on counts, 256 possible UMI's and poissonian counting statistics. The scripts to generate the count files can be found here https://github.com/vertesy/TheCorvinas/tree/master/Python/MapAndGo2:https://github.com/vertesy/TheCorvinas/blob/master/Python/MapAndGo/Readme_MapAndGo.md. Spike-in RNAs were discarded and not included in further analysis. Transcript counts of all assayed plates (technical replicates of injury types) were combined into one matrix for downstream analysis. Downstream analysis of the combined samples transcript read counts was performed using Seurat (v2.3.4). Transcript counts matrix was imported using CreateSeuratObject function (min.cells=25, min.genes=250) and low quality cells were discarded with FilterCells with the following thresholds: a minimum of 500 and maximum of 3500 genes, a maximum of 10% of mitochondrial genes and a minimum of 1000 and maximum of 15000 UMIs). The filtered dataset was log-normalised using a scale factor 10000 and a set of ˜2800 genes were used for linear dimension reduction (FindVariableGenes, x.low.cutoff=0.05, x.high.cutoff=3, y.cutoff=0.5). Unwanted sources of variation were removed by regressing out UMI number per cell, percentage of mitochondrial reads per cell. Clustering analysis was performed with Seurat's FindClusters function (reduction.type=“pca”, dims. Use=1:50, resolution=0.5, save.SNN=TRUE) and a non-linear dimensional reduction using RunTSNE (reduction.use=“pca”, dims.use=1:50, perplexity=30, tsne.method=“Rtsne”). Seven cell clusters were identified. To identify cluster biomarkers either FindAllMarkers (logfc.threshold=0.25, test.use=“wilcox”) or FindMarkers (min.pct=0.20) functions were used to compare all clusters against each other or cluster specific comparisons. tSNE, feature plots and heatmaps were created using Seurat's TSNEPlot, FeaturePlot and DoHeatmap functions. Trajectory analysis was performed using partition-based graph abstraction (PAGA)⁸⁶, which is part of the scanpy package (v 1.4.5.2.dev6+gfa408dc)⁸⁷, In summary, a neighbourhood graph of data points (cells) was created using scanpy.pp.neighbors function⁸⁸ (starting from the top 50 principal components, as for PCA and UMAP and number of neighbours of 35) to then run PAGA using scanpy's scanpy.tl.paga function with the previously identified clusters as groups. Finally, PAGA cell embedding was represented using a force-directed layout (FDL) initialised with PAGA coordinates using scanpy's scanpy.tl.draw_graph function (Force Atlas 2 layout).

Pseudotime analysis was performed using diffusion pseudotime (DPT)⁸⁹. Scanpy's scanpy.tl.dpt function was run using the previously defined cluster 3 (composed of cells from the uninjured timepoint), as root. PAGA and FDL plots were generated using scanpy's sc.pl.paga sc.pl.paga_path (n_avg=25) and scanpy.pl.draw_graph functions. Metascape (http://metascape.org) was used to carry out biological process enrichment analysis using the differentially expressed genes of each cluster.

### Tissue Specific Loss-of-Function Zebrafish Mutations

The Tol2-flanked transgene Tg(4×UAS:NLS-Cas9, cryaa:EGFP)^{gl36Tg} (referred to as Tg(4×UAS.NLS-Cas9, cryaa:EGFP)) and Tg(4×UAS:NLS-Cas9, cmlc2:RFP)^{gl37Tg} (referred to as Tg(4×UAS NLS-Cas9, cmlc2:RFP)) were assembled by Gateway cloning. These constructs were microinjected with Tol2 mRNA into the required background. Tg(4×U1AS:NLS-Cas9, cryaa:EGFP) was introduced to the Tg(mpeg1:GAL4FF/UAS:NfsB-mCherry) background while Tg(4×UAS:NLS-Cas9, cmlc2:RFP) was introduced into both Tg(mpx:KALTA4/UAS:NfsB-mCherry) and Tg(pax7b:Gal4; UAS:GFP) backgrounds. Co-segregation of the three transgenes through F0 and F1 backcrosses onto this background was achieved by selecting embryos with the appropriate fluorescence (Tg(mpeg1:GAL4FF/UAS:NfsB-mCherry/4×UAS.NLS-Cas9,cryaa:EGFP): sorted for red macrophages confirming the presence of the mpeg1:GAL4FF transgene and green eyes confirming the presence of the cryaa:EGFP marker gene linked to the 4×UAS:NLS-Cas9 (this line is referred to as mpeg1-Cas9). Tg(mpx:KALTA4/UAS:NfsB-mCherry/4×UAS:NLS-Cas9,cmlc2:RFP): sorted for red neutrophils and a red heart (this line is referred to as mpx-Cas9). Tg(par7b:Gal4; UAS:GFP/4×UAS:NLS-Cas9, cmlc2:RFP): sorted for green muscle stem/progenitor cells and a red heart (this line is referred to as pax7b-Cas9)). For gene editing experiments, embryos in crossed from either F2 or F3 generation adults were used. For nampta and ccr5 targeting, the gRNA or dual gRNAs combination used to generate mutants (see above) were used. For namptb targeting a dual gRNAs combination was used. These gRNAs were generated using, namtb crRNA_1, 5′-tttctctgaccaaacacgcaAGG-3′ and namptb crRNA_2, 5′-gttgacctgtgaacgtgataGGG-3′. The nampta individual gRNA and ccr5 and namptb dual gRNAs efficiencies were tested in whole embryo gene editing and exhibited to have between 89-100% mutational efficiency. To induce tissue specific gene editing 3 nL of gRNA mix (3 μL of 3 μM gRNA (if dual gRNA used 1.5 μL of each gRNA)+0.5 μL of 2% phenol red+1.5 μL 0.1 M KCl mix) was injected into the cell of one-cell stage embryos.

### Visualisation and Quantification of NAD/NADH

In vivo two-photon excited fluorescence of larval zebrafish NADH was measured on an Olympus FVMPE-RS upright multi-photon microscope using a 25×/1.05 water immersion objective. A wavelength of 810 nm and a 450/70 bandpass filter was used. The same wavelength and a 610/70 bandpass filter were used to detect mCherry fluorescence. A galvo scanner was used to generate high-resolution data sets while an 8 kHz resonant scanner was used for time-lapse imaging to minimise phototoxic effects.

In vitro total NAD⁺ and NADH levels and their individual levels (to determine their ratio) were measured using the NAD/NADH-Glo™ assay (Promega) following supplier instructions. Total NAD/NADH measurement: The assay was carried out on macrophages sorted from macrophage specific nampt knockout larvae (mpeg1-Cas9+nampt gRNA injected) and control (mpeg1-Cas9) larvae. At 2 dpf control larvae were soaked in either the NAMPT enzymatic inhibitor GMX1778 (10 μM) (Sigma-Aldrich) or NAMPT's rate limiting enzyme catalyses product, nicotinamide mononucleotide (NMN) (100 μM) (Sigma-Aldrich). These were used as additional controls to identify the assay's detection range. At 3 dpf mpeg1⁺ macrophages from each group were sorted into white, flat bottom 96 well plates (Costar) containing 50 μL PBS at 2000 cells/well density. Cells were incubated in 50 μL of NAD/NADH Glo™ detection reagent. After 1 h incubation, luminescence was determined in a microplate reader (BMG PHERAstar; gain 3600, 1 s integration time). Each point of measurement represents the average luminescence reaction measured in relative luminescence units (RLU). Measuring NAD⁺ and NADH individually: The assay was carried out on FACS sorted mCherry⁺ macrophages isolated from Tg(mpeg:GAL4FF/UAS:NfsB-mCherry) 4 dpf uninjured larval trunks and dissected myotomal needle stick injured larval wound sites at 1 dpi (5 dpf) and 2 dpi (6dpf). cells were sorted at 5000 cells/well density into white, flat bottom 96 well plates (Costar) containing 50 μL PBS. Following manufactures instructions two separate reactions were carried out to eliminate one of either NAD⁺ or NADH and the remaining metabolite was detected using 100 μL of NAD/NADH Glo™ detection reagent. After 2 h incubation, luminescence was determined as before and the NAD⁺/NADH ratios were calculated based on levels of NAD⁺ and NADH.

### RT-PCR

Total RNA from FACS sorted pax3a⁺ cells were extracted using TRIzol™ Reagent (Thermo Fisher). cDNA was synthesized using the iScript™ Advanced cDNA Synthesis Kit (Bio-Rad) following manufacturer's instructions. For RT-PCR, 10 μl reactions were set up using GoTaq® Green Master Mix (Promega). Primers used to amplify zfccr5, 5′-TTATAACCAAGAGACATGTCGGCG-3′ and 5′-ACCCAGACGACCAGACCATT-3′. Primer pair designed to cross a 388 bp intron so that cDNA and genomic DNA (gDNA) templates would result in bands of 191 bp and 579 bp, respectively. The cycling protocol was performed as follows: Initial denaturation at 95° C. for 2 min followed by 25 cycles of denaturation at 95° C. for 30 sec, annealing at 58° C. for 1 min and extension at 72° C. for 45 sec followed by a final extension of 5 min at 72° C. Cycling protocol provided allowance to amplify both cDNA and gDNA templates enabling identification of genomic DNA contaminants.

Total RNA was extracted and cDNA synthesised from 4 dpf zebrafish larval tails of different genotypes (mutant, heterozygous and homozygous siblings were identified by genotyping larval heads from heterozygous in-crosses of nampta^{+/pc41} ccr5^{+/pc42}). RT-PCR was carried out using the following primer pairs, all of which were designed to cross an intron; zfnampta_mutant_RT, 5′-CCGACTCCTACAAGGTCACAC-3′ and 5′-TTGACTTTTCGGGGCTTGGT-3′ (wild type amplicon 115 bp); zfccr5_mutant_RT, 5′-TTGAGCTGTTATAACCAAGAGACA-3′ and 5′-GAGGGAAAATTAAGCTCAGAAGG-3′ (wild type amplicon 657 bp); zfactc1b_housekeeping_RT, 5′-TTGACAACGGCTCCGGTATG-3′ and 5′-GCCAACCATCACTCCCTGAT-3′ (amplicon 110 bp).

### Maximum-Likelihood Phylogenetic Tree Analysis

A putative Ccr5 orthologue in zebrafish was identified by BLAST and orthology assessed by phylogenetic analysis. Amino acid sequences were aligned by MUSCLE and trimmed using GBLOCKS. PHYML was used to generate a maximum likelihood tree using the JTT model for protein evolution (as inferred using ProTest v3.4.2). Trees were visualised using iTOL (v4.3)(⁷⁰⁻⁷⁶)

### Statistical Analysis

Embryos for each experimental treatment group were assigned randomly and the groups were blinded to the experimenter prior to analysis. All experiments were performed with a minimum of 3 independent biological replicates; exact numbers have been indicated in figures. Statistical analysis was carried out using Prism version 7.0c (GraphPad Software, Inc.). Data was analysed using Student's unpaired two-tailed t-test when comparing two conditions and Analysis of Variance (ANOVA) with Tukey's post-hoc analysis when comparing multiple conditions.

### NAMPT402-491 Production and Purification

Final protein concentration: 280 μg/mL

Expected size: 11 kDa

Observed size on gel: 14-15 kDa (data not shown)

12% SDS-PAGE:

Lane 1: NAMPT402-491

Lane 2: MW marker (kDa)

Amino Acid Sequence:

MSYVVTNGLG VNVFKDPVAD PNKRSKKGRL SLHRTPAGNF
VTLEEGKGDL EEYGHDLLHT VFKNGKVTKS YSFDEVRKNA
QLNIEQDVAP HHHHHHH

Number of amino acids: 97 (this example includes a septaHis C-terminal moiety) Molecular weight: 10977.26

Extinction coefficients (used to calculate:

This protein does not contain any Trp residues. Experience shows that this could result in more than 10% error in the computed extinction coefficient.

Extinction coefficients are in units of M-1 cm-1, at 280 nm measured in water.

Ext. coefficient 4470

Abs 0.1% (=1 g/1) 0.407 (value used to adjust Nanodrop reading)

Preparation Protocol

Plasmid preparation and bacterial transformation:
NAMPT402-491 DNA sequence was subcloned in pET22b(+) vector (plasmid map here)
Vector transformed in competent E. Coli (BL21DE3 strain) by heat shock at 42° C. for 30 s
Transformed bacteria were spread on an ampicillin/agar plate and a single colony was selected.

Protein Production:
5 mL starter culture in LB+100 μg/mL ampicillin incubated overnight
After incubation the starter culture was diluted 1:100 in LB+100 μg/mL ampicillin and expanded until OD600 reached 0.7 (˜4 h)
Induction for 4h with 1 mM IPTG
Bacterial culture spined at 4000 g for 20 min and pellet resuspended in lysis buffer (PBS+50 mg lysozyme+1% Triton X-100+2.5 U benzonase+10 mM MgCl2+1 tablet Complete EDTA-free protease inhibitor).
Sonication for 4×30 s and incubation at 4° C. for 30 min with gentle shaking
Lysate was spined at 14′000 g for 30 min and supernatant filtered before protein purification.

Protein Purification:
Lysate loaded on HisTap HP (GE healthcare)
LPS removed with Triton X-114 0.1% wash
Bacterial chaperones removed with 2 mM ATP+10 mM MgCl2 wash
Other contaminants removed with 20 mM imidazole wash
Protein eluted with imidazole gradient from 20 mM to 500 mM
Fractions containing the pure protein were pooled and dialyzed overnight in PBS
Protein concentrated using a 3 kDa amicon filter.

The present description is further illustrated by the following examples, which should not be construed as limiting in any way.

### EXAMPLES

Live imaging of the collective cellular response to tissue injury remains a long-term goal of the regenerative medicine field. In an attempt to attain this goal previously developed zebrafish models of tissue injury were used where the optical accessibility of the larvae allows the application of non-invasive techniques to assay repair in real time (Gurevich D. B. et al. Asymmetric division of clonal muscle stem cells co-ordinate muscle regeneration in vivo. Science (2016)) (3).

This approach has been applied herein to regenerating skeletal muscle in order to determine the cellular and molecular events that define regeneration in vivo. Transgenic zebrafish reporter lines fluorescently tagging wound-present cellular components were subject to acute injury, enabling the location and response dynamics of individual wound-occupying cells to be correlated to the stem cell compartment during muscle repair.

Two injury paradigms were used: a focal laser ablation model that severs 2-6 fibres per injury and a needle stab model that consistently injures 2 adjacent myotomes. Systematic characterisation of different cellular behaviours within the regenerating niche in both models highlighted the directed and obligate association between specific macrophage populations and muscle stem cells during regeneration. Distinct modes of high-resolution imaging coupled with in vivo cell tracking defined these interactions with high temporal and spatial resolution.

### Specific Macrophage Subsets are Primed to Respond to Trauma

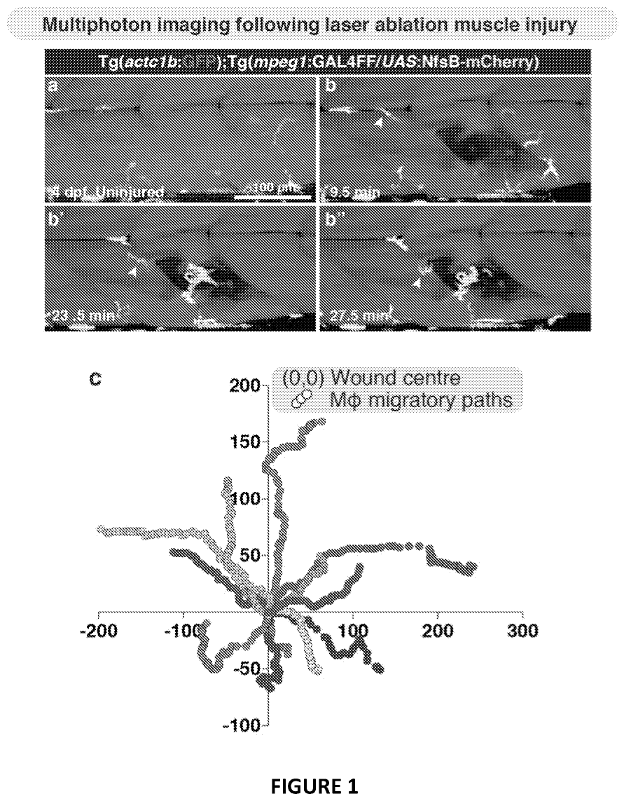

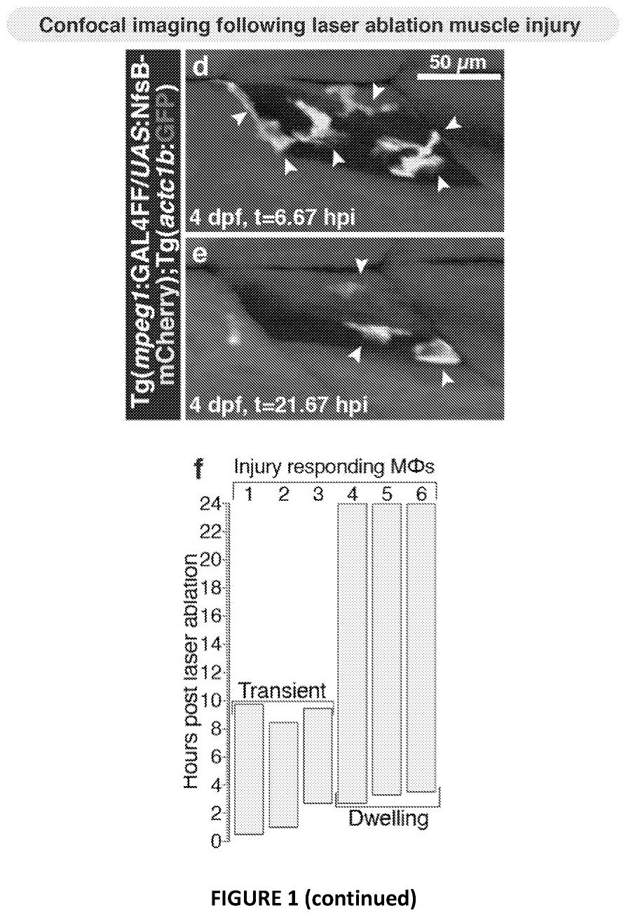

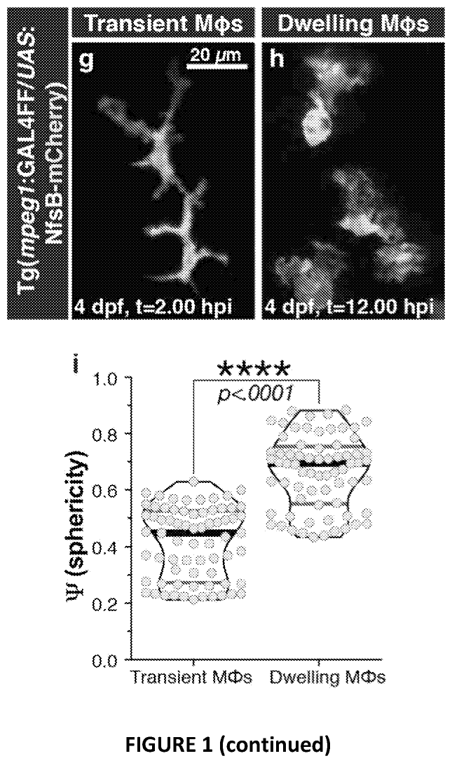

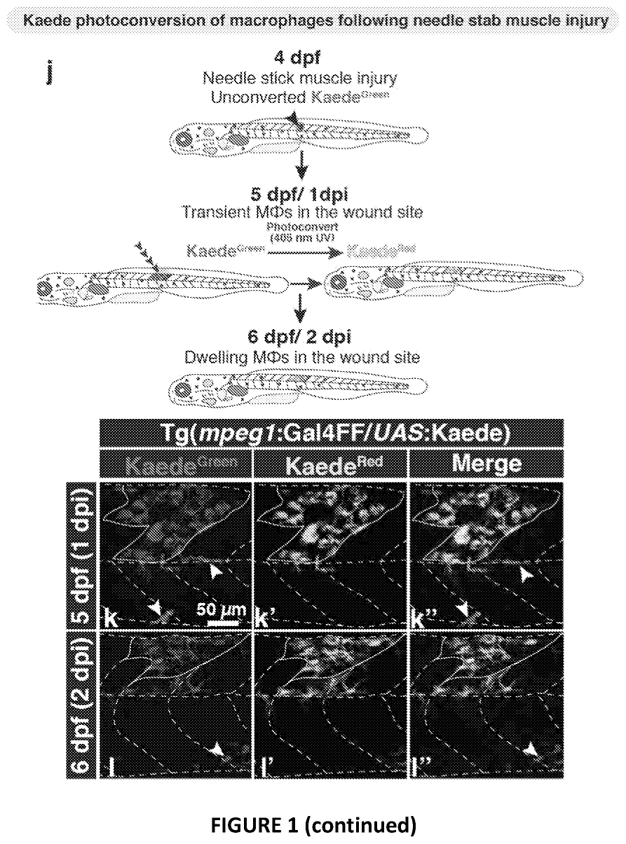

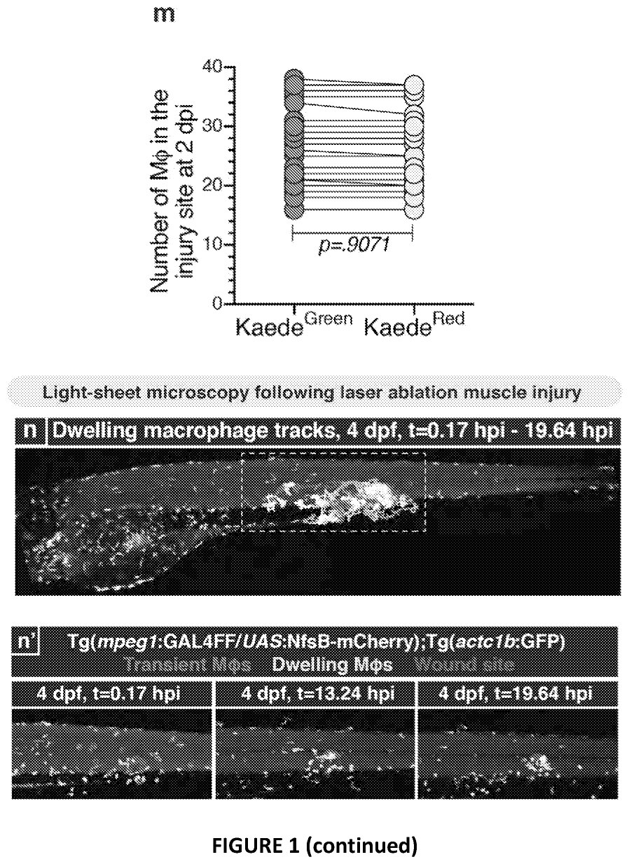

Initially, multiphoton imaging was utilised to capture the response prior to, and immediately following, laser ablation muscle injury. The compound transgenic line Tg(actc1b:GFP); Tg(mpeg1:GAL4FF/UAS:NfsB-mCherry), in which differentiated muscle expresses green fluorescent protein (GFP) and macrophages are labelled by mCherry fluorescence, allowed the wound region to be defined while observing the kinetics of injury-responsive macrophages (FIG. 1a-c, Supplementary video. 1 (not provided)). Following muscle fibre laser ablation injury, 34±2% of macrophages (MΦs) in the vicinity of the wound (MΦs within a 260 μm radius from the centre of the laser ablation, a region encompassing 2 myotomes on either side of the injury, n=4 injuries) displayed rapid, active and directed migration towards the wound site (average distance travelled: 128.31±68.03 μm, average velocity: 0.149±0.040 μmsec-1, n=30 MΦs assayed in n=4 larvae, FIG. 1c, Supplementary Video 1 (not provided)). That not all the injury-proximate macrophages responded to the lesion suggested the potential existence of specific macrophage subsets that are primed to respond to trauma.

Next, injury-migrated macrophage dynamics within the boundaries of the wound site were examined at a cellular resolution following muscle fibre laser ablation. High-resolution continuous confocal imaging demonstrated that peak macrophage numbers within the wound site were reached at 2.50±0.42 hours post injury (hpi) (n=8 injuries), following which, no additional macrophages migrated into the wound (FIG. 1d-f, Supplementary Video. 2 (not provided)).

Of the total injury-responding macrophages, 51.11±1.83% (n=8 injuries) remained within the wound site up to 24 hpi (FIG. 1e-f). These long-term injury-located macrophages are referred to as ‘dwelling’. In contrast, macrophages that exit the wound site do so prior to 10.48±1.19 hpi and were consequently designated ‘transient’ (FIG. 1d, f).

High-resolution confocal imaging revealed that these two macrophage populations display morphological differences, with the transient cells exhibiting an obvious stellate appearance while the dwelling cells adopt a more spherical form (dwelling MΦs exhibited a 0.248±0.022 higher sphericity value when compared to transient MΦs, FIG. 1g-i, Supplementary Video. 3 (not provided)). This transient to dwelling macrophage transition occurred irrespective of the magnitude of injury, but scaled temporally with wound size (51.6% of needle stab injury-responsive MΦs went on to dwell by 2 days post injury (dpi), n=20, FIG. 5 a-d).

The lineage relationship between distinct subsets of wound-active macrophages remains an issue of considerable debate within the field. To establish whether dwelling macrophages are a subset of the original pool of injury-responsive macrophages or resultant from an alternative macrophage migratory wave, a Tg(mpeg1:Gal4FF/UAS:Kaede) transgenic line in which macrophages are labelled with the photoactivatable fluorescent protein Kaede was utilised. One-day post needle stick muscle injury, transient macrophages present within the wound site were photoconverted to distinguish them from macrophages external to the injury (n=20, FIG. 1j-k″). On re-imaging the injury site one day later, all the dwelling macrophages within the wound site exhibited the photoconverted form of Kaede (FIG. 1l-m). This indicates that dwelling macrophages are a subset derived from the initial transient injury-responding macrophage population, and not the result of another, temporally distinct, migration.

The respective contributions of tissue resident and recruited macrophages during muscle repair is a further area of active investigation. Coupling light-sheet technology with the larval zebrafish model provides a unique opportunity to examine this area, as it allows the capture of individual macrophage migratory events at a whole organism vantage during the repair process. As such individual macrophages were tracked throughout the entire zebrafish larvae from 10 min to 25 hpi following laser ablation muscle fibre injury in the Tg(actc1b:GFP) and Tg(mpeg1:GAL4FF/UAS:NfsB-mCherry) transgenic line by light-sheet microscopy (Supplementary Video. 4 (not provided)). Retrospective tracking of injury-located macrophages revealed that all macrophages that proceeded to assume a dwelling phenotype migrated from the vicinity of the wound site, and as such are tissue resident rather than migratory in nature (n=4 injuries, FIG. 1n-n′). These observations suggested that there is regionalised anatomical constraint to the activation of wound-responsive macrophages in the zebrafish larvae. This localised response is reminiscent of the previously described pro-homeostatic “cloaking” function of tissue-resident macrophages, whereby they segregate micro-lesions of various tissues, including muscle, to prevent superfluous neutrophil migration. However, in the context of the muscle injuries, why some wound-proximate macrophages refrain from migrating into the injury site remains an open question.

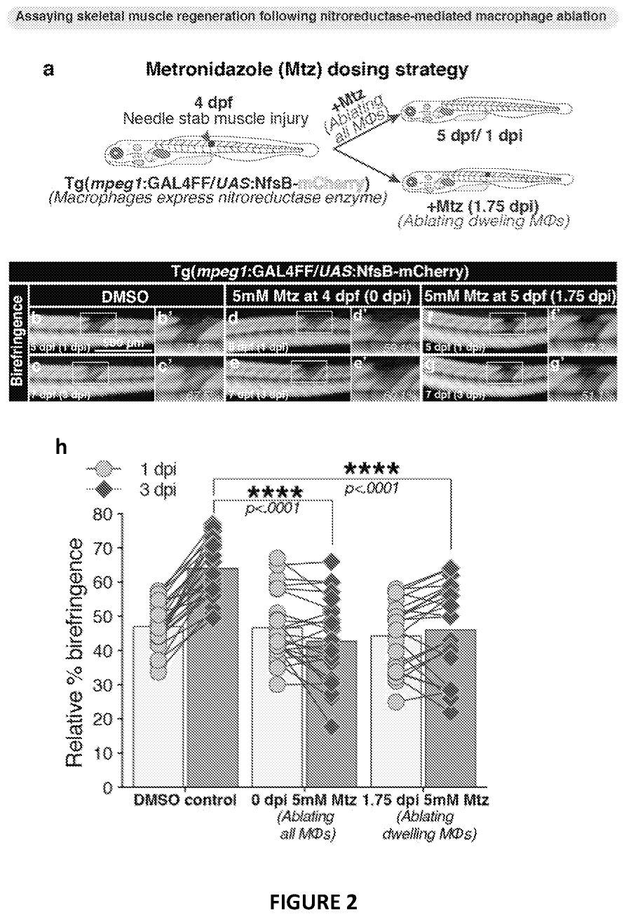

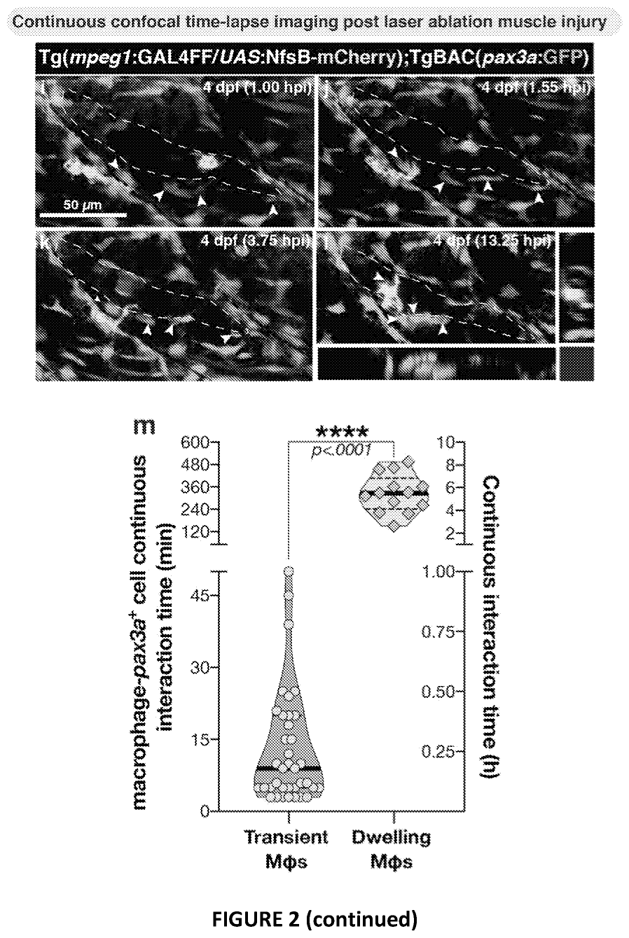

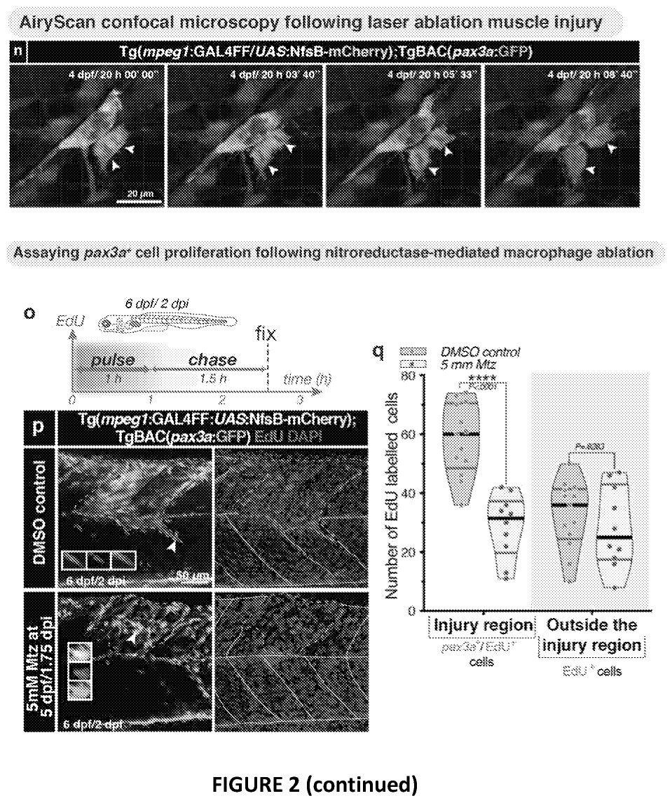

Macrophages indispensable for efficient skeletal muscle regeneration, but there is also an explicit requirement for the dwelling macrophage subpopulation for injury repair As the residency of dwelling macrophages occurred in the same temporal window as stem cell mediated muscle repair, it was next examined if they were required for this process by comparing muscle regeneration in the presence and absence of the transient and dwelling macrophage populations. The Tg(mpeg1:GAL4FF/UAS:NfsB-mCherry) transgenic line allows timely, temporally-controlled, nitroreductase-mediated genetic ablation of macrophages by the addition of metronidazole (Mtz) at discrete time points during muscle regeneration (FIG. 2a. FIG. 5e-f, Supplementary Video 5 (data not provided)). Mtz addition in this transgenic line leads to efficient cell death, with macrophage ablation visible following 3 h of treatment. After 10-13 h, the majority of trunk present macrophages have been ablated and their cell corpses cleared from the larvae (FIG. 5g, Supplementary Video. 5). Furthermore, this ablation strategy did not alter other innate immune cell responses to muscle wounding at 2 dpi (FIG. 5 h-i). Regeneration following needle stick muscle injury was assessed by birefringence imaging of the wound site at key milestones over the course of the repair process (n=24 per group, FIG. 2b-h). The pseudo-crystalline array of muscle sarcomeres confers it an intrinsic birefringence, making uninjured muscle appear bright against a black background when observed using polarised light, enabling non-invasive visualisation of muscle integrity (Berger, et al. (2012)). Addition of Mtz at the time of injury (to ablate all injury responding macrophages) and Mtz treatment at 1.75 dpi (to preferentially ablate dwelling macrophages) both resulted in a significant regeneration deficit (FIG. 2b-h, FIG. 5 g′). In order to establish that the observed repair deficit was exclusive to macrophages and not a result of Mtz induced toxicity or a response to a general innate immune deficit, a previously characterised Tg(mpx:KALTA4/UAS:NfsB-mCherry) transgenic line was used which allows nitroreductase-mediated genetic ablation of neutrophils via an identical strategy used to ablate macrophages. Neutrophils are the other key cellular component of the innate immune response mounted by larval zebrafish to wounding and Mtz treatment at the time of injury, targeted at ablating all injury responsive neutrophils failed to disrupt muscle regeneration (FIG. 5 j-o). Hence, not only are macrophages indispensable for efficient skeletal muscle regeneration, but there is also an explicit requirement for the dwelling macrophage subpopulation for injury repair.

Failure of repair evident in macrophage-deficient settings does not result from ineffective debris clearance, but rather via the lack of an as-yet uncharacterised pro-myogenic function. Wound debris clearance is mediated by a number of cell types, but largely attributed to phagocytic macrophages. Absent or ineffective clearance might be one explanation for the lack of regeneration observed when macrophage populations are ablated early in the regenerative process. Four separate approaches were undertaken to assay wound debris clearance when all injury responding macrophages were ablated. Firstly, larvae were stained with fluorescently conjugated phalloidin to detect the actin cytoskeleton of remnant necrotic skeletal muscle fibres in the injury site (FIG. 6a). Secondly, the Tg(ubi:secAnnexinV-mVenus) transgenic line was utilised to identify apoptotic cells within the injury zone (FIG. 6 b-c). Thirdly, LysoTracker fluorescent vital dye was used to visualise acidic membranous organelles and the autophagic process (FIG. 6 d-e) and lastly Haematoxylin and Eosin staining was performed to monitor removal of dead and dying fibres in the wound site (FIG. 6 f). Accumulation of cellular debris in the injury site of macrophage-ablated larvae compared to un-ablated controls was not observed in any of these independent approaches (FIG. 6 a-f). These data suggest that the failure of repair evident in macrophage-deficient settings does not result from ineffective debris clearance, but rather via the lack of an as-yet uncharacterised pro-myogenic function.

Macrophages maintained highly dynamic membrane contacts with the stem cells, enveloping them with continuous and repetitive membrane extensions—macrophage-stem cell associations maybe essential for stem cell proliferation. Given that the position that dwelling macrophages occupy within the repair niche was topographically similar to that which had been identified as containing dividing muscle stem and progenitors cells (FIG. 1e), next, the spatial and temporal relationship between these two populations of cells was examined during muscle regeneration. Long-term confocal time-lapse imaging following laser ablation muscle injury was performed in the compound Tg(mpeg1:GAL4FF/UAS:NfsB-mCherry); TgBAC(par3a:GFP) transgenic line which labels both macrophages and pax3a expressing muscle stem/progenitor cells (FIG. 2i-l, Supplementary Video. 6 (data not provided)). The pax3a:GFP reporter is particularly useful in this context as it is expressed in both the muscle stem cell compartment as well as the dividing progenitor population. Following injury, transient macrophages and pax3a⁺ cells both responded and migrated simultaneously to the wound. However, par3a⁺ cells migrated independently of transient macrophages, displaying distinct migration kinetics, taking residence in their niche at the edge of the wound by 10.25±1.99 hpi (n=5 injuries, FIG. 2i-l, FIG. 7d-f, Supplementary Video. 6 not provided). Following their transition from a transient to dwelling macrophage phenotype, dwelling macrophages associated with the pax3a cells lining the wound edge at 11.17±1.13 hpi and displayed continuous interactions with adjacent stem cells over the course of 5.38±1.79 h (FIG. 2l-m, Supplementary Video. 6 (data not provided)). These interactions are distinct in their nature and duration when compared to the short-lived interactions between transient macrophages and pax3a⁺ cells that are maintained on average for 12.86±11.95 min (n=37 interactions, FIG. 2 i-k, m). This set of interactions was also recapitulated in analyses using the Tg(me1:mCherry-2A-KALTA4/UAS:NfsB-mCherry) muscle stem cell marking line³ and the myogenic stem/progenitor marker gene trap line in which GAL4FF is integrated into the par7b gene (referred to as, Tg(par7b:GAL4FF))¹¹ and used to drive Tg(UAS:NfsB-mCherry) (FIG. 7a-c, Supplementary video. 9).

### A Direct Role for a Specific Macrophage Subset in Controlling Muscle Regeneration.

In order to better visualise these cellular interactions, high-resolution live imaging using AiryScan confocal microscopy was carried out on co-located dwelling macrophages and pax3a+ muscle stem/progenitor cells (FIG. 2n, Supplementary Video. 7 (data not provided)). Sustained associations were observed between the co-resident macrophages and stem cells. Macrophages maintained highly dynamic membrane contacts with the stem cells, enveloping them with continuous and repetitive membrane extensions (n=10 injuries, FIG. 2n, FIG. 7g, Supplementary Video 7, 8 (data not provided)). Following these protracted physical interactions, the associated pax3a+ muscle stem cell invariably underwent cell division (FIG. 2n, Supplementary Video 7, 8 (data not provided)). These macrophage-stem cell interactions were further validated by correlative light and electron microscopy (CLEM), which confirmed that the two cell types displayed tight membrane appositions in three-dimensional XYZ planes (FIG. 8a-d). Following these protracted physical interactions, the associated pax3a⁺ muscle stem cell invariably underwent cell division (FIG. 2n, FIG. 7g, Supplementary Video. 7, 8). On average, dwelling macrophages interacted with a specific stem/progenitor cell for 5.42±1.72 h (n=10 interactions) prior to the associated stem cell undergoing division. This is comparable to the time a subset of dwelling macrophages maintains uninterrupted interactions with wound-responsive muscle stem cells (5.38±1.79 h, FIG. 2l-m), highlighting that once a dwelling macrophage initiates stem cell interactions it ceases upon stem cell division. Crucially, no muscle stem cell divisions were observed in the absence of prolonged interactions with co-resident dwelling macrophages (n=26 wound associated stem cell divisions imaged in n=10 independent larvae). 71% of proliferation-inducing macrophages maintained long-term associations with a single muscle stem cell, 29% simultaneously associate with two stem cells and no macrophages were identified to maintain more than two concurrent long-term stem cell interactions prior to cell division (n=9 stem cell divisions). Following stem cell proliferation, the macrophage and daughter myoblasts migrate away from each other, with the macrophage taking on a myoseptal localisation 1.94±0.94 h following stem cell division. During this relocation, 44% of macrophages display associations over 1.45±0.69 h with one or both daughter myoblasts, while the remainder avert such associations (n=9 stem cell divisions, a FIG. 7h, Supplementary Video. 10). Intriguingly, the nature of these interactions between muscle stem cells and wound-dwelling macrophages are phenotypically reminiscent of dendritic cell-T cell immunological synapses within the lymph nodes that lead to T cell activation and proliferation. Collectively, these data suggest that macrophage-stem cell associations maybe essential for stem cell proliferation.

To assay the requirement of dwelling macrophages to stimulate stem cell proliferation EdU labelling in myotome needle stick injured larvae was performed where dwelling macrophages had been ablated (FIG. 2o-q). In control larval wound sites at 2 dpi, all EdU⁺ proliferative cells corresponded to pax3a⁺ myogenic stem/progenitor cells. Ablation of dwelling macrophages severely and significantly reduced the number of proliferating pax3a+ muscle stem cells within the injury site, reducing the number of EdU positive cells to homeostatic levels present within uninjured regions (51% decrease in proliferating pax3a+ muscle stem cells, n=13, FIG. 2p-q). Collectively, these results reveal a surprisingly direct role for a specific macrophage subset in controlling muscle regeneration, demonstrating that a proportion of wound-attracted macrophages form a transient stem cell niche. Ablation of this niche-specific macrophage subset leads to a severe reduction in the number of myogenic progenitors present within the injury site, and a consequent muscle regeneration deficit (FIG. 2f-h).

Unsupervised clustering identified a uniform population (cluster 3) from uninjured tissue, cluster 2 in transitory macrophages and many discrete clusters of injury specific dwelling macrophage subtypes. To establish the nature of injury-responsive macrophage populations, single-cell RNA-sequencing (scRNA-seq) was carried out on injury-located macrophages isolated from discrete phases of the regenerative process. Following needle stick skeletal muscle injury of Tg(mpeg1:mCherry) larvae, the wound region was dissected out at 1, 2 and 3 dpi and, mCherry expressing macrophages were FACS sorted (FIG. 3a). Macrophages sorted from uninjured larvae were also included in the analyses. Unsupervised clustering identified seven discrete clusters of macrophage subtypes (FIG. 3b). Of note, cells in all clusters expressed the pan-leukocyte marker L-plastin (lymphocyte cytosolic protein 1, Icp1) and the pan-macrophage marker cd163, validating their macrophage identity (FIG. 3e, FIG. 9a, p Table 1). Unsupervised clustering identified eight discrete clusters (cluster 0-7) of macrophage subtypes (FIG. 3b). This analysis revealed greater macrophage heterogeneity than previously described during muscle, or any other regenerative scenario¹⁴⁻¹⁹ (FIG. 3b). We next compared the time-point at which the injury-responsive macrophages were isolated, versus the identified clusters overlaid on a UMAP scatter plot, to explore if injury times corresponded to specific individual clusters (FIG. 3c). Macrophages from the uninjured larvae formed a temporally uniform cluster (Cluster 3, FIG. 3d, Supplementary Table. 2). Furthermore, unsupervised clustering of macrophages specifically from uninjured larvae revealed no further complexity, re-affirming their homogenous nature. This lack of predetermination in the uninjured macrophage population is intriguing given that only a proportion of muscle resident macrophages respond by migrating to the injury. It suggests that the ability to respond to wound-derived cues is an acquired state, at least at the level of gene expression. The majority of transient macrophages (1 dpi) also clustered together (Cluster 1, FIG. 3d, Supplementary Table. 2), suggesting a systematic initial activation of the macrophages that migrate into the injury. The remaining 6 clusters were composed of dwelling macrophages (2-3 dpi), highlighting their heterogeneous nature (Cluster 0, 2, 4, 5, 6 and 7, FIG. 3d, Supplementary Table. 2). We next undertook transcriptomic trajectory and pseudotime analyses to better understand the potential lineage relationship between the identified dwelling macrophage clusters. These analyses revealed that there are two ‘mature-dwelling’ macrophage subtypes represented by cluster 2 and 6. All other clusters have trajectories that suggest they are transitory subtypes from which these mature subsets arise (FIG. 3g-k).

In mammals, activated macrophages have classically been defined through in vitro studies as a simple dichotomy between pro-inflammatory, or M1, macrophages and an alternatively activated, anti-inflammatory, or M2²⁰, macrophage pool. Although recent analyses clearly illustrate that this simple dichotomy of macrophage phenotypes does not represent in vivo macrophage diversity, specific markers can be used to identify pro-inflammatory and anti-inflammatory subtypes. An analysis of genes differentially expressed between each of the clusters revealed that cluster 2 macrophages were specifically enriched for mammalian anti-inflammatory subtype markers, including arginase 2 (arg2), matrix metalloproteinase 9 (mmp9) and matrix metalloproteinase 13 (mmp13)²¹⁻²³ (53.4%, 100% and 98.6% of Cluster 2 present cells expressing arg2, mmp9 and mmp13, respectively, FIG. 3e, Supplementary Table. 3).

Since all Cluster 2 cells expressed mmp9 (FIG. 3e, k), we assessed its spatiotemporal expression following injury both by in situ hybridisation to the endogenous mRNA (FIG. 3l, o) and via the use of a transgenic line that expresses EGFP under the control of the mmp9 promoter (TgBAC(mmp9:EGFP)²⁴, FIG. 3m-n, FIG. 10a and Supplementary Video. 11). These analyses revealed that mmp9 expression was up-regulated from 2 dpi onwards in the muscle stem cell-associated dwelling macrophage population present in the wound site (FIG. 3l-m, o). Quantitative analysis identified that at 2 dpi, 71.09±12.05% (n=11) of mpeg⁺-dwelling macrophages in the wound site were mmp9⁺ (FIG. 3m-o), a value range that is in line with the scRNA-seq analysis which identified 59% of all 2 dpi macrophages express mmp9 (FIG. 3f). The number of mmp9⁺ macrophages is also consistent with the number of wound site-present, dwelling, macrophages that go on to actively interact with muscle stem cells (72.92±20.83%). Furthermore, the majority of the remaining dwelling macrophages in the wound site at 2 dpi, expressed markers specific to cluster 6 (15.10±12.13% of cells are Pou2f3⁺/mpeg⁺ and 23.36±12.85% of cells are Prox1a⁺/mpeg⁺. FIG. 9b-g). This observation strongly correlates with the scRNA-seq trajectory and pseudo-time analyses described above (FIG. 3g-k), which define cluster 2 and 6 as the two ‘mature-dwelling’ macrophage subtypes present during late stage wound regeneration. Furthermore, In contrast to cluster 2 cells, the vast majority of cluster 6 cells were not located adjacent to pax3a⁺ muscle stem/progenitor cells (at 2 dpi, 12±10% of Pou2f3⁺/mpeg⁺ cells (n=8) and 9±7% of Prox1a⁺/mpeg⁺ cells (n=10) were located adjacent to wound site-present pax3a⁺ cells), highlighting, that this subset does not actively interact with muscle stem cells during their proliferative phase in wound repair.

In order to more directly implicate Cluster 2 macrophages in the regulation of regenerative muscle stem cell proliferation, we specifically ablated these cells during the repair process, using an alternative mmp9 promoter-driven transgenic line that expressed the nitroreductase enzyme fused to EGFP (TgBAC(mmp9:EGFP-NTR)²⁴). These larvae were subjected to a needle stab muscle injury at 4 dpf (0 dpi). At 1.75 dpi larvae were treated with 5 mM Mtz, a dosing that specifically ablated 77.94% of mmp9⁺/mpeg⁺-macrophages present in the injury zone (FIG. 10b-d). Specific ablation of these cells led to a significant skeletal muscle repair deficit (FIG. 3p-q) and a severe reduction in muscle stem cell proliferation to levels similar to those evident upon mpeg⁺-dwelling macrophage ablation, documented above (FIG. 2f-h, FIG. 3r, FIG. 10e-g). Collectively, these analyses indicate that Cluster 2 contains a specific mmp9⁺ muscle stem cell-associated, dwelling macrophage subset required for muscle stem cell proliferation during muscle regeneration

Cluster 4 (re-classified as Cluster 2) macrophages express arg2, mmp9 and mmp13 and are specific muscle stem cell-associated dwelling macrophages. Cells in Cluster 4 (re-classified as Cluster 2) were enriched for mammalian M2 markers including arginase 2 (arg2), matrix metalloproteinase 9 (mmp9) and matrix metalloproteinase 13 (mmp13)) suggesting a dwelling macrophage-specific anti-inflammatory subtype (53.5%, 100% and 98.6% of Cluster 4 present cells express arg2, mmp9 and mmp13, respectively). Since all Cluster 4 (reclassified as cluster 2) cells expressed mmp9, its spatiotemporal expression was determined following injury and spatial restriction was observed to the wound site and up-regulation from 2 dpi onwards in the muscle stem cell-associated dwelling macrophage population (FIG. 3f-h). These analyses indicate that Cluster 4 (re-classified as cluster 2) contains a specific muscle stem cell-associated dwelling macrophage subset.

It is to be noted that the Cluster numbers for the macrophage clusters identified have been re-named as follows: previous version Cluster 3 (uninjured)--> current version Cluster 3; previous version Cluster 4 (mmp9+)--> current version Cluster 2; previous version Cluster 6 (pou2f3+/prox1a+)--> current version Cluster 6. The characterisation of these macrophage clusters (Clusters 1-8) as illustrated in the supplementary Tables allows for the selection and/or enrichment of macrophage clusters based on their differential gene marker expression profile/s, functional characterisation based, for example, on their differential gene expression profiles, and behaviour in vitro and in vivo, eg anti-inflammatory, differentiation promoting activity.

NAMPT-CCR5 signalling axis induces muscle stem cell proliferation in vivo. The list of genes encoding secreted pro-mitogenic molecules specifically up-regulated within Cluster 4 (re-classified as Cluster 2) compared to all other identified clusters (Extended Data Table. 1 (data not included) was assessed. This was matched with data sets comprising receptors known to be expressed on muscle stem/progenitor cells (Yahiaoui, L., Gvozdic, D., Danialou, G., Mack, M. & Petrof, B. J. CC family chemokines directly regulate myoblast responses to skeletal muscle injury. The Journal of Physiology 586, 3991-4004 (2008))(21), leading to the identification of a pairing between nicotinamide phosphoribosyltransferse (NAMPT/Visfatin/PBEF) and the chemokine receptor CCR5 (FIG. 3e). NAMPT is a multi-functional protein, which carries out a well-characterised role in cellular metabolism and NAD regeneration when localised intracellularly and a very poorly characterised role as a secreted factor. Its secreted form (secreted NAMPT (secNAMPT)) has been documented to function as a cytokine with reports of both physiological and pathological functions (Grolla, A. A., Travelli, C., Genazzani, A. A. & Sethi, J. K. Extracellular nicotinamide phosphoribosyltransferase, a new cancer metabokine. British journal of pharmacology 173, 2182-2194 (2016)(25)). In contrast to its intracellular function, information concerning the role of the secreted protein is contradictory and not well understood, with respect to both its mode of secretion as well as mechanism of action. Despite this lack of mechanistic insight, secNAMPT has been demonstrated to be pro-mitogenic, and is also documented as a regulator of a broad variety of regenerative capacities in distinct tissues (Grolla et al. (2016)). CCR5 has been suggested as a putative cell surface receptor for NAMPT (Van den Bergh, R. et al. Monocytes contribute to differential immune pressure on R5 versus X4 HIV through the adipocytokine visfatin/NAMPT. PloS One, e35074 (2012)(26)). Additionally, NAMPT has been demonstrated to alter the expression of myogenic regulatory factors in myoblasts in vitro. Consequently, we hypothesised that the NAMPT-CCR5 signalling axis promotes muscle stem cell proliferation in vivo.

To examine if the NAMPT/CCR5 signalling axis could regulate muscle stem cell-mediated repair in vivo, the spatiotemporal expression of the two nampt genes encoded in the zebrafish genome was determined, nampta (the homolog of the single mammalian Nampt-encoding gene and the orthologue specifically up-regulated within cluster 2) and namptb (a gene only present in fish, possessing a much lower homology to mammalian Nampt)³², within wound-dwelling macrophages in vivo, using a number of independent approaches. Firstly, we undertook in situ hybridisation analyses subsequent to larval muscle injury and observed that nampta was specifically up-regulated within the wound site at 2 and 3 dpi (FIG. 10h), whereas namptb was expressed at a constant low-level within the injury zone from 1-3 dpi (FIG. 13k). These observations are consistent with the scRNA-seq data, which identified only nampta as the gene up-regulated in cluster 2 (FIG. 3e, Supplementary Table. 3). Secondly, we identified a NAMPT antibody that recapitulated the endogenous developmental expression profile of nampta³² (FIG. 10k) and antibody staining was consequently performed in the compound Tg(mpeg1:GAL4FF/UAS.NfsB-mCherry); TgBAC(pax3a:GFP) transgenic line. These analyses confirmed that Nampt expression in the injury site is temporally up-regulated at 2 dpi (FIG. 10l-o) and that Nampt expression in the wound site is localised specifically to the stem cell-associated dwelling macrophages as well as the extracellular space of the injury site (FIG. 4a). Of the dwelling macrophages present in the injury site at 2 dpi, 72±16% (n=8 injuries) were Nampt⁺/mpeg1⁺ (a value range in line with the scRNA-seq analysis that identified 58% of 2 dpi macrophages expressing nampta, FIG. 3f). This quantification is similar to both the percentage of dwelling macrophages that go on to interact with muscle stem cells (72.92±20.83%) and the percentage of mpeg⁺ macrophages that also express mmp9⁺ in the wound site at 2 dpi (71.09±12.05%). All Nampt⁺/mpeg1⁺ double positive cells were associated with pax3a⁺ muscle stem/progenitor cells (FIG. 4a). Furthermore, whole mount immunohistochemistry analyses on regenerating larvae revealed a reduction in Nampt levels following macrophage, but not neutrophil-specific ablation, again reinforcing that the source of secreted Nampt within the muscle injury is macrophage-derived (FIG. 10n-o).

To monitor the levels and source of increased NAMPT activity during the regeneration of muscle in vivo, we made use of the key physiological role of intracellular NAMPT in catalysing the rate-limiting process in the NAD salvage pathway. Increased NAMPT activity has been documented to elevate levels of intracellular NADH. NADH exhibits endogenous fluorescence with an excitation peak at 365 nm that is amenable to examination by two-photon excitation fluorescence microscopy. We therefore developed an assay to examine NADH levels in vivo, and used this as a proxy to estimate cellular Nampt levels in real time within the wound milieu (Hong, S. M. et al. Increased nicotinamide adenine dinucleotide pool promotes colon cancer progression by suppressing reactive oxygen species level. Cancer science 110, 629-638 (2019). At 2 dpi, dwelling macrophages alone specifically expressed high levels of NADH within the wound site (FIG. 10i, Supplementary Video. 12) and quantifying the levels of NAD⁺ and NADH within isolated wound-located macrophages, using a luminescence-based assay, confirmed the in vivo based observations (dwelling MΦs have a 0.6781±0.1650 lower NAD⁺/NADH ratio compared to transient MΦs, FIG. 10j). These findings confirm that the increased Nampt production observed in the wound site during regeneration occurs specifically within the dwelling macrophages and highlights their role as the primary source of Nampt in the wound site. Furthermore, the high levels of NADH and consequent reduced NAD⁺/NADH ratio in dwelling macrophages may result from a metabolic shift, specifically in the cluster 2/mmp9⁺-dwelling macrophage subtype. There is mounting evidence that a macrophage's metabolic state is linked with its activation (Watanabe, R. et at. Glucose metabolism controls disease-specific signatures of macrophage effector functions. JCI insight 3 (2018), and an analysis of our scRNA-seq dataset reveals a potential glycolytic shift in the mnp9⁺ subset (FIG. 9h-i). Alternatively, high Nampta expression levels may arise as a consequence of Nampta secretion occurring at levels high enough to deplete intracellular stores, which in turn triggers an induction of high levels of nampta expression within dwelling macrophages.

Selective signalling of NAMPT via the CCR5 receptor is required to induce myoblast proliferation. To better dissect the molecular basis of NAMPT function on muscle stem cells in vitro assays were done in mammalian cell culture. Molecular reagents and recombinant proteins have been generated in mammalian systems that allows rigorous evaluation of the model of stem cell activation we have generated from our in vivo zebrafish-based observations. We first sought to establish if secNAMPT acted to regulate muscle stem cell proliferation through its putative receptor, CCR5. Since there is only a single report of a direct NAMPT-CCR5 interaction, we set out to independently evaluate NAMPT binding to CCR5. The binding of human recombinant NAMPT (hrNAMPT source 1 (hrNAMPT₍₁₎)) to the human recombinant CCR5 receptor (hrCCR5) was evaluated via an enzyme-linked immunosorbent assay (ELISA) approach. We found that hrNAMPT₍₁₎ binds hrCCR5 with a dissociation constant (K_{D}) of 172±18 nM (FIG. 11a), confirming previous findings³⁰. NAMPT is a highly evolutionary conserved protein, with human NAMPT sharing 96% and 88.24% identity with mouse NAMPT and zebrafish Nampta proteins, respectively. Since we utilise hrNAMPT in our mouse cell lines assays described below, we also assayed hrNAMPT's affinity for mouse recombinant CCR5 (mrCCR5) by means of a competitive ligand-binding assay with the cognate ligand, CCL4. Here, mouse recombinant CCL4 (mrCCL4) demonstrated a half maximal inhibitory concentration (IC₅₀) of 34.4±2.2 nM, revealing both hrNAMPT's strong affinity for mrCCR5 and its binding to the receptor via the same sites utilised by CCL4 (FIG. 11b).

Next, in vitro murine cell culture models were used to determine if NAMPT initiates a pro-proliferative signalling cascade on muscle stem cells and assess if the NAMPT-CCR5 interaction yields functional modulation of this process. For this we utilised mouse C2C12 myoblasts. These myoblasts do not appear to actively secrete NAMPT, as C2C12 secretome data either fails to detect or very lowly detects this protein. However, we determined that these cells do express the CCR5 receptor at a density (2,470±441 molecules/cell (n=6)) that is in line with previously documented physiologically relevant levels of CCR5 (Gilliam, B. L., Riedel, D. J. & Redfield, R. R. Clinical use of CCR5 inhibitors in HIV and beyond. Journal of translational medicine 9, S9 (2011). Having validated the suitability of the C2C12 system for our assays, two human recombinant NAMPT protein sources (hrNAMPT₍₁₎ and hrNAMPT₍₂₎) were applied to C2C12 myoblasts, and proliferation assayed by means of EdU incorporation. Both sources of NAMPT resulted in comparable and significant dose dependent increases in myoblast proliferation (FIG. 11f). In order to uncouple the intracellular and extracellular roles of NAMPT during proliferation, these myoblasts were treated with GMX1778, a highly specific and potent inhibitor of NAMPT's enzymatic function. Drug treatment had no negative effect on the basal level of C2C12 proliferation in culture and also did not affect the increased myoblast proliferation produced following exogenous NAMPT supplementation (FIG. 11f), highlighting that NAMPT's pro-proliferative role is not reliant on its intracellular enzymatic function. The enhanced proliferative response observed following NAMPT supplementation could be recapitulated in C2C12 cells by the addition of the canonical CCR5 ligands, CCL8/MCP-2 and CCL4/MIP-1β, but not by the CCR2 ligand CCL2/MCP-1 (FIG. 11f). In addition, this proliferative response was blocked in the presence of the dual CCR2/CCR5-antagonist, cenicriviroc (CVC) and CCR5 selective-antagonist maraviroc (MVC), but not in the presence of the CCR2-selective antagonist PF-4136309 (PF) (FIG. 11f). Collectively, this data highlights that selective signalling of NAMPT via the CCR5 receptor is required to induce myoblast proliferation. Next, we turned to an in vitro multi-cell co-culture system to examine the relationship between macrophages and NAMPT-mediated proliferation. We assessed the proliferation of isolated embryonic mouse PAX7⁺ muscle stem cells in three culture conditions: embryonic mouse primary myoblast only (MB), and co-cultures of mouse myoblast with either mouse macrophages (MB+MΦ) or 3T3 mouse fibroblast cells (MB+3T3). The mouse macrophage cell line utilised is known to express high levels of NAMPT, while 3T3 fibroblast cells do not naturally secrete NAMPT in culture. In these experiments, the presence of macrophages promoted satellite cell proliferation and a response was not elicited when macrophages were replaced in the co-culture by 3T3 cells Furthermore, addition of hrNAMPT was able to increase the proliferation of satellite cells in MB and MB+3T3 culture conditions to a comparable level to that evident in MB+MΦ cultures), an effect that was abolished in the presence of CVC. Addition of CVC also reduced satellite cell proliferation in MB+MΦ culture conditions to levels evident in MB alone or MB+3T3 cultures. These results highlight that the macrophage driven increase in satellite cell proliferation was primarily mediated by secNAMPT, acting through the CCR5 receptor. Collectively, these observations show that macrophage secreted NAMPT is a critical pro-proliferative signal that activates the CCR5 receptor present on satellite cells to stimulate muscle regeneration.

Subsequently, these findings were validated in vivo. The zebrafish ccr5 orthologue was found to be expressed on 2 dpi FACS sorted pax3a⁺ myogenic stem/progenitor cells by RT-PCR), and administered CVC and MVC to larval zebrafish to block Ccr5 signal propagation following needle stick muscle injury. Using birefringence imaging a highly significant regeneration deficit was identified in the drug-administered larvae. This was not due to effects on macrophage migration kinetics or a block in macrophages transitioning from a transient to a dwelling state, both of which are phenotypically wild-type upon drug administration. Rather, a reduction in the proliferation of pax3a⁺ myogenic stem cells present in the wound site was observed that was similar in magnitude to that observed post-ablation of dwelling macrophages.). Collectively, these data are consistent with macrophage-secreted nampt acting in a Ccr5-dependent manner to activate muscle stem cell proliferation during regeneration in vivo.

Unconditional NAMPT gene knockout in mice results in embryonic lethality. To circumvent this eventuality confounding our analyses, a macrophage specific loss-of-function mutagenesis system was developed. A stable macrophage-specific expression of Cas9 from a Tg(UAS:NLS-Cas9) transgene coupled to a Tg(mpeg1:Gal4FF) transgene was generated. By delivering a nampt guide RNA (gRNA) by microinjection, a durable macrophage specific nampt gene editing was achieved and subsequently assayed the role of role macrophage-derived Nampt in muscle repair (FIG. 4i, FIG. 8d). Immunostaining for Nampt revealed a visible reduction in Nampt expressing cells present in the wound site following needle stick muscle injury in the nampt gRNA injected larvae. Furthermore, quantification of NAD+/NADH levels in isolated macrophages functionally validated macrophage specific nampt loss-of-function using this macrophage specific gene editing approach.

Following needle stick muscle injury, nampt gRNA injected larval macrophages responded by migrating to the injury zone and locating within the wound boundaries from 1 to 3 dpi. However, these nampt-deficient macrophages failed to induce appropriate cell proliferation and regeneration at the injury site highlighting a specific requirement for functional macrophage-derived Nampt to ensure appropriate regeneration. Collectively, the in vitro cell culture analyses, together with our in vivo chemical inhibition and macrophage-specific nampt loss-of-function studies demonstrate, a requirement for macrophage-derived NAMPT to stimulate muscle stem cell proliferation in a CCR5-dependent manner.

Next, it was determined that addition of exogenously applied NAMPT could accelerate regeneration in a mouse model of volumetric muscle loss, an injury paradigm usually refractory to normal stem cell mediated repair processes and an area of unmet clinical need (Quarta, M. et al. Bioengineered constructs combined with exercise enhance stem cell-mediated treatment of volumetric muscle loss. Nature Communications 8, 1-17 (2017)(39)). This analysis confirmed that exogenously-applied NAMPT also acts in the context of an adult model of injury and validated the mammalian cell culture based results in vivo. Strikingly, delivery of hrNAMPT in a fibrin hydrogel, but not a fibrin only control hydrogel was able to fully restore muscle architecture when applied to wound site, indicating that exogenously supplied NAMPT protein stimulates muscle repair in the context of an acute injury of adult mammalian muscle in a similar manner to the results we describe above for zebrafish larvae. It also reinforces that the secreted form of NAMPT is acting in both these in vivo settings.

It is proposed herein that even this simplest of stem cell systems requires a complex interaction with a range of cellular contexts during repair in vivo, and that the innate immune system, in particular the macrophage, is a key modulator of the regenerative process. The present of specific defined macrophage subset is demonstrated herein, and a surprising complexity of macrophage phenotypes present during wound repair, acting as a transient stem cell niche that directly regulates muscle stem cells through the provision of mitogenic stimuli, specifically the NAMPT/CCR5 axis. Collectively, the results described herein show that providing specific macrophage-derived signals required for muscle stem cell proliferation, such as those identified here, provides an avenue to achieve better myoblast and tissue stem cell-based therapy outcomes.

These results were supported by the following further experimental data and are illustrated in the Figures.

Next, a multi-cell type, in vitro co-culture system was used to examine the relationship between macrophages and NAMPT-mediated proliferation. The proliferation of isolated embryonic mouse PAX7⁺ muscle stem cells in three culture conditions was assessed: embryonic mouse primary myoblast only (MB), and co-cultures of mouse myoblast with either mouse macrophages (MB+MΦ) or 3T3 mouse fibroblast cells (MB+3T3). The mouse macrophage cell line utilised (Maf/DKO) is known to express high levels of NAMPT, but we sought to quantitate the specific levels of NAMPT secretion under our culture conditions. Following 16 h of culture, Maf/DKO macrophages secreted 5.24±0.67 ng/ml of NAMPT into the supernatant (FIG. 11). Additionally, these cells did not actively secrete CCR5's cognate ligands (CCL3, CCL4 and CCL5) into the supernatant (FIG. 11d-e). In contrast, 3T3 fibroblast cells do not naturally secrete NAMPT in culture. In these experiments, we observed that the presence of macrophages promoted satellite cell proliferation (FIG. 4k, FIG. 11g), a response not elicited when macrophages were replaced in the co-culture by 3T3 cells (FIG. 4k, FIG. 11g). Furthermore, addition of hrNAMPT was able to increase the proliferation of satellite cells in MB and MB+3T3 culture conditions, to a level comparable to that evident in MB+MΦ cultures (FIG. 4k, FIG. 11g), an effect that was abolished in the presence of CVC as well as MVC, and maintained in the presence of PF4 (FIG. 4k, FIG. 11g). These results highlight that the macrophage-driven increase in satellite cell proliferation was primarily mediated by secNAMPT, acting through the CCR5 receptor. Interestingly, hrNAMPT did not increase satellite cell proliferation in the presence of macrophages suggesting that there is a maximum threshold rate of proliferation and this may be modulated by receptor saturation. Moreover, CCL4 supplementation in the co-cultures mirrored the results of NAMPT addition, reaffirming that NAMPT's proliferative effect is mediated by CCR5. Collectively, these observations support the hypothesis that macrophage-secreted NAMPT is a critical pro-proliferative signal that activates the CCR5 receptor present on satellite cells to stimulate muscle regeneration.

Subsequently, these findings were validated in vivo. the zebrafish ccr5 orthologue was expressed on 2 dpi FACS isolated pax3a⁺ myogenic stem/progenitor cells by RT-PCR (FIG. 11h-j), and administered CVC and MVC to larval zebrafish immediately following needle stab muscle injury (4 dpf) to block Ccr5 signal propagation. Using birefringence imaging a highly significant regeneration deficit was detected in the drug-administered larvae (FIG. 11r-s). This was not due to effects on macrophage or neutrophil migration kinetics or a block in macrophages transitioning from a transient to a dwelling state, all of which are phenotypically wild-type upon drug administration (FIG. 11k-p). Rather, a reduction in the proliferation of par3a⁺ myogenic stem cells present in the wound site was observed that was similar in magnitude to that observed post-ablation of dwelling macrophages (FIG. 2f-h, FIG. 11t-u). Furthermore, these results were confirmed by long-term time lapse imaging of CVC administered, muscle laser ablated, larvae which displayed no discernible deficits in macrophage and stem cell migration into the injury zone or macrophage transition into a dwelling state (MΦs start to dwell at 10.20±0.91 hpi, n=6 injuries). Dwelling macrophages started to affiliate with muscle stem cells by 11.80±0.57 hpi (n=6) and interacted identically to control macrophages maintaining continuous, uninterrupted associations. However, in contrast to untreated larvae, dwelling macrophages remained associated with its target stem cell until experimental end point (18 h, n=6) and did not terminate as in control settings, where these interactions cease and the two cell types move apart following stem cell division (FIG. 11q, Supplementary Video. 13). In this manner, stem cell division may act as a trigger for the separation of a macrophage and its associated stem cell, allowing the macrophage to progress to its next target stem cell. Collectively, these data consistently demonstrate that Ccr5-mediated signalling is a necessity to activate muscle stem cell proliferation during regeneration in vivo.

To provide further evidence of the role of the Nampta/Ccr5 signalling axis in regulating the pro-regenerative, macrophage-derived, muscle stem cell niche, germline mutations were generated by CRISPR-Cas9-mediated gene editing, in both the namtpa and ccr5 genes and assayed muscle regeneration in homozygous mutants (FIG. 12a-c, k-m). In contrast to mice, where unconditional NAMPT gene knockout results in embryonic lethality⁵⁰, larval zebrafish are able to survive nampta germline knockout. This is likely due to the fact that namptb can functionally compensate and fulfil, at least in part, the protein's enzymatic role in the NAD⁺ salvage pathway³² that is crucial for survival. Both mutants presented no comparable difference in the number of injury-responsive macrophages or dwelling macrophages compared to their wild type or heterozygote siblings (FIG. 12d-e, n-o). Furthermore, dwelling macrophages from both mutants went onto interact with wound site-located muscle stem cells (FIG. 12f, p). Both nampta and ccr5 homozygous mutants documented a significant regeneration deficit using birefringence analyses following needle stab muscle injury (FIG. 12g-h, q-r). Furthermore, in both cases this deficit was due to a significant decrease in proliferating muscle stem cells within the injury zone (FIG. 12i-j, s-t).

To determine if it is specifically macrophage-derived Nampta that is required for the pro-regenerative activity, we developed a novel macrophage-specific loss-of-function mutagenesis system (A. I. I, Z. Zhang, V. Pazhakh, H. R. Manley, E. R. Thompson, L. C. Fox, S. Yerneni, P. Blombery, G. J. L, in preparation). Stable macrophage-specific expression of Cas9 protein was generated from a Tg(4×UAS:NLS-Cas9) transgene coupled to a Tg(mpeg1:Gal4FF) transgene. By delivering a nampta guide RNA (gRNA) by microinjection, durable macrophage-specific nampta gene editing with sequence disruptions were evident in and around the nampta gRNA target site of gene-edited macrophages (FIG. 13a). Immunostaining for Nampta revealed a visible reduction in Nampt-expressing cells present in the wound site following needle stab muscle injury in the nampta-gRNA injected larvae (FIG. 13b). Furthermore, quantification of NAD⁺/NADH levels in isolated macrophages functionally validated macrophage specific nampta loss-of-function using this macrophage-specific gene editing approach (FIG. 13c).

Using this validated, lineage specific, gene-editing approach the role of macrophage-derived Nampta in muscle repair was assessed using our standardised muscle injury paradigms (FIG. 4b). Following needle stick muscle injury, nampta-gRNA injected larval macrophages responded by migrating to the injury zone, transitioning to a dwelling subtype and actively interacting with muscle stem cells in the injury zone (FIG. 13d-g). However, these nampta-deficient macrophages failed to induce appropriate cell proliferation and regeneration at the injury site (FIG. 4d-g), highlighting a specific requirement for functional macrophage-derived Nampta to ensure appropriate regeneration. Additional control experiments were conducted for the mpeg-Cas9 experiments and new experiments inhibiting NAMPTs enzymatic activity and demonstrating that it does not have a negative effect on muscle stem cell proliferation Macrophage-specific namptb knockdown larvae, in contrast, undergo significant muscle regeneration, albeit at a reduced rate to that evident in control injured larvae (Extended Data FIG. 13l-n). Furthermore, neutrophil-specific deletion of nampta using a Tg(mpx1:KALTA4); Tg(4×UAS:NLS-Cas9) transgene combination failed to result in any deficit in muscle regeneration (FIG. 13o-q), reaffirming macrophages as the source of wound site-present Nampta. Lastly, we examined if Nampt's pro-regenerative cytokine activity was independent of its enzymatic function in vivo, as we had determined in our in vitro based assays. Inhibiting Nampt's intracellular enzymatic function by the addition of GMX1778 to regenerating larvae in a tight-temporally controlled window, from the point macrophages start to dwell, did not alter muscle stem cell proliferation (FIG. 10p-q). This confirmed our in vitro findings that Nampt's secreted form governs its proliferative functionalities. Collectively, our in vitro cell culture analyses, together with our in vivo chemical inhibition and macrophage-specific nampta loss-of-function studies, demonstrate a requirement for macrophage-derived NAMPT to stimulate muscle stem cell proliferation in a CCR5-dependent manner (FIG. 14j).

Next, we designed experiments to confirm that the Ccr5 receptor was specifically required on muscle stem cells during the regenerative process to stimulate their proliferation. To undertake these analyses, the above described pax7b:GAL4FF line was used that is expressed in wound-present muscle stem cells (FIG. 7c). Using this pax7b promoter to drive expression of 4×UAS:NLS-Cas9 transgenic line enabled muscle stem cell-specific ccr5 knockdown using a highly efficient dual gRNA combination. This muscle stem cell-lineage specific ablation of ccr5 did not alter the number of transient and dwelling macrophages in the injury zone and further, did not affect the dwelling macrophage-stem cell associations (FIG. 13h-j). These gene-edited larvae displayed a significant regeneration deficit following needle stab muscle injury (FIG. 4c-e) and significant muscle stem cell proliferation deficits in the wound site (FIG. 4h-i). Both defects were similar in magnitude to those evident in germline ccr5 mutants (FIG. 12k-t) and larvae in which Ccr5 activity was inhibited chemically (FIG. 11r-s). Collectively, these data reveal that muscle stem cell-Ccr5 activity is required during muscle regeneration to induce stem cell proliferation.

Next the effect of exogenous-NAMPT addition to larval zebrafish following needle stab muscle injury was examined. Since previous work has demonstrated the potential of secreted NAMPT to inhibit neutrophil apoptosis⁵¹ and thereby alter inflammatory dynamics, the innate immune cell response to NAMPT supplementation was examined. In our acute injury setting no alternations in immune cell dynamics or altered wound site lysosomal activity was observed upon exogenous-NAMPT treatment (FIG. 14a-c). NAMPT supplementation, however, did lead to a highly significant, 30.70±4.26% increase in muscle stem cell proliferation within the wound site (FIG. 4j, FIG. 14d). Furthermore, NAMPT supplementation also functioned to rescue wound site proliferation in macrophage-ablated larvae, even acting to increase the proliferation 10.96±3.55% above the response in a control setting (FIG. 4j, FIG. 14d). However, NAMPT failed to rescue the proliferation deficit when supplemented in conjunction with CVC (FIG. 4j, FIG. 14d). Interestingly, while the canonical Ccr5 ligand, CCL8, was able to enhance the wound site proliferative response to a similar level as that of NAMPT (on average 28.34±4.48% higher than control), it also increased cell proliferation external to the site of injury by 43.03±4.46% compared to control (FIG. 4j, FIG. 14d). This highlights NAMPT as having a specific function on wound-site present muscle stem cells distinct from a generalised proliferative response elicited by Ccr5 activation.

Next, we investigated if addition of exogenously applied NAMPT could accelerate regeneration in a mouse model of volumetric muscle loss, an injury paradigm usually refractory to endogenous-stem cell mediated repair processes and an area of unmet-clinical need⁵². This analysis confirmed that exogenously applied NAMPT could also act in the context of an adult model of injury and validated the murine cell culture based results in vivo. Strikingly, delivery of hrNAMPT into the muscle defect via a fibrin hydrogel, but not a fibrin only control hydrogel, was able to fully restore muscle architecture when applied to the wound site (FIG. 4l-o). On average, treatment with a single dose of hrNAMPT (0.5 μg) at the point of injury led to a 3.276±0.4926 mm² increase in average muscle area and a 34.76±9.32% decrease in average fibrotic area. NAMPT addition in the VML injury model results in a significant increase in both the total number and proportion of proliferating PAX7⁺ satellite cells (FIG. 4p-r, FIG. 14e) and a significant increase the number of centrally nucleated de novo muscle fibres (FIG. 4s-t) but does so without inducing any significant alteration to the immune cell profile of the regenerate (FIG. 14h-i). Angiogenesis within the wound occurred at levels similar to that described for other pro-regenerative approaches in VML injuries^{52,53} (Extended Data FIG. 14f-g), suggesting that angiogenesis simply scales with the level of regeneration evident in NAMPT-treated injuries. However, a more directed mode of action of NAMPT and CCR5 in stimulating angiogenesis cannot be formally ruled out, particularly given that both these proteins have previously been shown to induce endothelial cell proliferation in a number of different contexts⁵⁴⁻⁵⁶. Collectively, these findings indicate that exogenously supplied NAMPT protein stimulates muscle repair in the context of an acute injury of adult mammalian muscle in a similar manner to the results we describe above for zebrafish larvae. It also reinforces the finding that it is the secreted form of NAMPT that is active in both these in vivo settings.

### NAMPT Fragments and Derivatives

In one embodiment, dimers are formed by adding a cysteine residue at the N-terminus (with or without a linker, for example GGS or repeat of GGS). The cysteine can also be the one naturally present in the NAMPT sequence:

CSYVVTNGLGINVFKDPVADPNKRSKKGRLSLHRTPAGNFVTLEEGKGDL
EEYGQDLLHTVFKNGKVTKSYSFDEIRKNAQLNIELEAAHH

Human:

SYVVTNGLGINVFKDPVADPNKRSKKGRLSLHRTPAGNFVTLEEGKGDLE
EYGQDLLHTVFKNGKVTKSYSFDEIRKNAQLNIELEAAHH

Mouse:

SYVVTNGLGVNVFKDPVADPNKRSKKGRLSLHRTPAGNFVTLEEGKGDLE
EYGHDLLHTVFKNGKVTKSYSFDEVRKNAQLNIEQDVAPH

A C-terminal fragment of NAMPT stimulates muscle stem cell proliferation. As described above NAMPT, is a large homodimeric intracellular enzyme which acts as cytokine when released in the extracellular milieu. However, the NAMPT domain responsible for cytokine activity is unknown. Re-examining the crystal structure of NAMPT determined that the terminal structure of the NAMPT C-terminus highly resembles classic CCR-binding chemokines (such as CCL2), due to its size and structure (C-terminus α-helix and β-sheets) (FIG. 9a). Moreover, the domain extends out from the core protein structure potentially facilitating receptor binding. Thus, the C-terminus of NAMPT was recombinantly reproduced and its ability to compete with NAMPT binding to CCR5 and to stimulate satellite cell proliferation was tested. Remarkably, this fragment, which is termed herein a “cytokine finger” (cif), inhibits NAMPT binding to CCR5 (IC50=21.5 nM, FIG. 9b) and stimulates satellite cell proliferation in a dose-dependent manner, inducing growth at similar levels to full-length NAMPT (FIG. 9c). Collectively, these data demonstrate that the C-terminus cif domain is responsible for the NAMPT's muscle cytokine activity.

NAMPT cytokine derivatives for clinical applications. NAMPT derivatives are engineered with a range of modifications to improve delivery and enhance receptor-mediated activity. These forms are serially assayed for clinically useful characteristics such as increased or optimal receptor binding affinity, in vitro cell based proliferation and in vivo in zebrafish or mammalian muscle regeneration assays. Tissue targeting is clearly of great value in multiple modes of administration and it is proposed to include tissue directed moieties to enhance delivery and appropriate retention in the target tissue. NAMPT or NAMPT derivatives may further be administered in carriers such as nanocarriers known in the art to be able to provide tissue targeting through their composition or functionalised structure.

Dimerising the NAMPT cytokine finger to increase receptor affinity. NAMPT is naturally a homodimer and known CCR5-binding chemokines are dimers and can form multimers (trimers, tetramers and above through oligomerisation) that modulate receptor binding-affinity and signalling. In one embodiment, NAMPTcif is dimerized to increases its binding affinity to CCR5 (FIG. 9d). In one approach the single Cysteine residue naturally present at the N-terminus of NAMPTcif is used to force its dimerization. The binding affinity of dimeric NAMPTcif for CCR5 are tested with ELISA and with surface plasmon resonance (SPR) assays. The activity of dimeric NAMPTcif to promote mouse primary satellite cells proliferation is tested. Quiescent satellite cells are sorted from fresh muscle as used above, that relies on negative and positive cell surface markers (CD31−, CD11b−, CD45−, TER119−, Sca1−, CD34+, CD106+) specific for a subset of satellite cells that have been shown to have the highest self-renewal characteristics in vivo. Cells are cultured in vitro and the efficacy of NAMPT derivatives to stimulate proliferation of these primary muscle stem cells is determined. The regenerative capacity of the dimeric, and multimeric NAMPTcif and functional derivatives is tested in the described zebrafish regeneration assay.

Addition of ECM- and/or syndecan-binding motifs is used as one preferred approach to optimize delivery and increase tonic signalling on CCR5 (see Mochizuki et al Nat. Biomed Engineering 2019) (See FIG. 14). Several proteins bind syndecans such as laminins. One particular syndecan binding moiety is the globular domain of the laminin-α chain having the sequence RKRLQVQLSIRT (SB). Addition of binding molecules may be by synthetic means or using recombinant approaches, as known in the art. Illustrative, non-limiting ECM binding moieties comprises RGD, or YGISR, YIGSR, GFOGER, IKVAV, and GEFYFDLRLKGK.

One suitable model for testing function derivatives is an established model of cardiotoxin induced muscle injury. Cardiotoxin is a myonecrotic agent that kills muscle cells without disrupting muscle ECM, providing an important model to test the ECM binding motif containing NAMPT variants. It is the most commonly used model in assaying muscle stem cell activation as it leaves the majority of stem cells intact. The standard models are used where an intramuscular injection of cardiotoxin into the Tibialis anterior (TA) muscle is assayed for restoration of lost fibres. The final model tested are the mdx mouse, which has also been established at ARMI. The mdx model is used to test both the stem cell activating potential of NAMPT derivatives as well their anti-fibrotic capability, as the mdx model exhibits chronic muscle fibrosis as well as muscle degeneration. Muscle regeneration is tested at established time points using standard histological assays described above.

The satellite cell is archetypal of a unipotent tissue resident stem cell that occupies a specific anatomical niche within a differentiated tissue. Decades of research have revealed the extraordinary capacity of this system to effectively coordinate muscle repair in response to a wide variety of insults. Despite this demonstrated regenerative capacity, transplantation of isolated muscle stem cells has yet to provide therapeutic impact, and pro-regenerative treatments that stimulate muscle stem cells are entirely lacking at this juncture. The data disclosed herein suggest that even this simplest of stem cell systems requires a complex interaction with a range of cellular contexts during repair in vivo, and that the innate immune system, in particular the macrophage, is a key modulator of the regenerative process. Demonstrated herein, is the concept of a specific macrophage subset, one amongst a surprising complexity of macrophage phenotypes present during wound repair, acting as a transient stem cell niche that directly regulates muscle stem cells through the provision of mitogenic stimuli, specifically the NAMPT/CCR5 axis (FIG. 14j). Thus, providing specific macrophage-derived signals required for muscle stem cell proliferation, such as the signals identified here, provide an avenue to achieve better myoblast-based therapy outcomes.

| First | Second base | | | | | | | |
|---|---|---|---|---|---|---|---|---|
| base | T | | C | | A | | G | |
| T | TTT | Phe (F) | TCT | Ser (S) | TAT | Tyr (Y) | TGT | Cys (C) |
| | TTC | Phe (F) | TCC | Ser (S) | TAC | | TGC | |
| | TTA | Leu (L) | TCA | Ser (S) | TAA | STOP | TGA | STOP |
| | TTG | Leu (L) | TCG | Ser (S) | TAG | STOP | TGG | Trp (W) |
| C | CTT | Leu (L) | CCT | Pro (P) | CAT | His (H) | CGT | Arg (R) |
| | CTC | Leu (L) | CCC | Pro (P) | CAC | His (H) | CGC | Arg (R) |
| | CTA | Leu (L) | CCA | Pro (P) | CAA | Gln (Q) | CGA | Arg (R) |
| | CTG | Leu (L) | CCG | Pro (P) | CAG | Gln (Q) | CGG | Arg (R) |
| A | ATT | Ile (I) | ACT | Thr (T) | AAT | Asn (N) | AGT | Ser (S) |
| | ATC | Ile (I) | ACC | Thr (T) | AAC | Asn (N) | AGC | Ser (S) |
| | ATA | Ile (I) | ACA | Thr (T) | AAA | Lys (K) | AGA | Arg (R) |
| | ATG | Met (M) START | ACG | Thr (T) | AAG | Lys (K) | AGG | Arg (R) |
| G | GTT | Val (V) | GCT | Ala (A) | GAT | Asp (D) | GGT | Gly (G) |
| | GTC | Val (V) | GCC | Ala (A) | GAC | Asp (D) | GGC | Gly (G) |
| | GTA | Val (V) | GCA | Ala (A) | GAA | Glu (E) | GGA | Gly (G) |
| | GTG | Val (V) | GCG | Ala (A) | GAG | Glu (E) | GGG | Gly (G) |

**Note codons can be replaced, please see replacement table for substitution of codons

Replacement Codons:

| 1st | Second Letter | | | | | | | | 3rd |
|---|---|---|---|---|---|---|---|---|---|
| letter | U | | C | | A | | G | | letter |
| U | UUU | Phe | UCU | Ser | UAU | Tyr | UGU | Cys | U |
| | UUC | | UCC | | UAC | | UGC | | C |
| | UUA | Leu | UCA | | UAA | Stop | UGA | Stop | A |
| | UUG | | UCG | | UAG | Stop | UGG | Trp | G |
| C | CUU | Leu | CCU | Pro | CAU | His | CGU | Arg | U |
| | CUC | | CCC | | CAC | | CGC | | C |
| | CUA | | CCA | | CAA | Gln | CGA | | A |
| | CUG | | CCG | | CAG | | CGG | | G |
| A | AUU | Ile | ACU | Thr | AAU | Asn | AGU | Ser | U |
| | AUC | | ACC | | AAC | | AGC | | C |
| | AUA | | ACA | | AAA | Lys | AGA | Arg | A |
| | AUG | Met | ACG | | AAG | | AGG | | G |
| G | GUU | Val | GCU | Ala | GAU | Asp | GGU | Gly | U |
| | GUC | | GCC | | GAC | | GGC | | C |
| | GUA | | GCA | | GAA | Glu | GGA | | A |
| | GUG | | GCG | | GAG | | GGG | | G |

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

TABLE 3
Exemplary and Preferred Amino Acid Substitutions

| Original | | |
|---|---|---|
| residue | Exemplary substitutions | Preferred substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

In some embodiments Trp residues are substituted.

TABLE 4
KEY TO SEQUENCE LISTING

| SEQ ID NO: | DESCRIPTION | SEQUENCE |
|---|---|---|
| SEQ ID NO: 1 | Illustrative Human C- | SYVVTNGLGINVFKDPVADPNKRSK |
| | terminal fragment of | KGRLSLHRTPAGNFVTLEEGKGDLE |
| | NAMPT as CCR5 | EYGQDLLHTVFKNGKVTKSYSFDEI |
| | interacting peptide | RKNAQLNIELEAAHH |
| SEQ ID NO: 2 | Mouse C-terminal | SYVVTNGLGVNVFKDPVADPNKRS |
| | fragment of NAMPT | KKGRLSLHRTPAGNFVTLEEGKGDL |
| | as CCR5 interacting | EEYGHDLLHTVFKNGKVTKSYSFDE |
| | peptide | VRKNAQLNIEQDVAPH |
| SEQ ID NO: 3 | Example of Human | MNPAAEAEFNILLATDSYKVTHYKQ |
| | NAMPT as CCR5 | YPPNTSKVYSYFECREKKTENSKLR |
| | interacting | KVKYEETVFYGLQYILNKYLKGKV |
| | polypeptide | VTKEKIQEAKDVYKEHFQDDVFNEK |
| | | GWNYILEKYDGHLPIEIKAVPEGFVI |
| | | PRGNVLFTVENTDPECYWLTNWIET |
| | | ILVQSWYPITVATNSREQKKILAKYL |
| | | LETSGNLDGLEYKLHDFGYRGVSSQ |
| | | ETAGIGASAHLVNFKGTDTVAGLAL |
| | | IKKYYGTKDPVPGYSVPAAEHSTITA |
| | | WGKDHEKDAFEHIVTQFSSVPVSVV |
| | | SDSYDIYNACEKIWGEDLRHLIVSRS |
| | | TQAPLIIRPDSGNPLDTVLKVLEILGK |
| | | KFPVTENSKGYKLLPPYLRVIQGDG |
| | | VDINTLQEIVEGMKQKMWSIENIAF |
| | | GSGGGLLQKLTRDLLNCSFKCSYVV |
| | | TNGLGINVFKDPVADPNKRSKKGRL |
| | | SLHRTPAGNFVTLEEGKGDLEEYGQ |
| | | DLLHTVFKNGKVTKSYSFDEIRKNA |
| | | QLNIELEAAHH |
| SEQ ID NO: 4 | Example of Mouse | MNAAAEAEFNILLATDSYKVTHYK |
| | NAMPT as CCR5 | QYPPNTSKVYSYFECREKKTENSKV |
| | interacting | RKVKYEETVFYGLQYILNKYLKGKV |
| | polypeptide | VTKEKIQEAKEVYREHFQDDVFNER |
| | | GWNYILEKYDGHLPIEVKAVPEGSVI |
| | | PRGNVLFTVENTDPECYWLTNWIET |
| | | ILVQSWYPITVATNSREQKKILAKYL |
| | | LETSGNLDGLEYKLHDFGYRGVSSQ |
| | | ETAGIGASAHLVNFKGTDTVAGIALI |
| | | KKYYGTKDPVPGYSVPAAEHSTITA |
| | | WGKDHEKDAFEHIVTQFSSVPVSVV |
| | | SDSYDIYNACEKIWGEDLRHLIVSRS |
| | | TEAPLIIRPDSGNPLDTVLKVLDILGK |
| | | KFPVTENSKGYKLLPPYLRVIQGDG |
| | | VDINTLQEIVEGMKQKKWSIENVSF |
| | | GSGGALLQKLTRDLLNCSFKCSYVV |
| | | TNGLGVNVFKDPVADPNKRSKKGR |
| | | LSLHRTPAGNFVTLEEGKGDLEEYG |
| | | HDLLHTVFKNGKVTKSYSFDEVRKN |
| | | AQLNIEQDVAPH |
| SEQ ID NO: 5 | Human NAMPT cif | CSYVVTNGLGINVFKDPVADPNKRS |
| | monomer as base for | KKGRLSLHRTPAGNFVTLEEGKGDL |
| | dimeric or multimeric | EEYGQDLLHTVFKNGKVTKSYSFDE |
| | form, with, in this | IRKNAQLNIELEAAHH |
| | embodiment, an n- | |
| | terminal cysteine to | |
| | facilitate dimer | |
| | formation | |
| SEQ ID NO: 6 | Example of a Human | agctatgttgtaactaatggccttgggattaacgtcttcaa |
| | cDNA NAMPT cif | ggacccagttgctgatcccaacaaaaggtccaaaaagg |
| | fragment encoding | gccgattatctttacataggacgccagcagggaattttgtt |
| | sequence | acactggaggaaggaaaaggagaccttgaggaatatg |
| | | gtcaggatcttctccatactgtcttcaagaatggcaaggtg |
| | | acaaaaagctattcatttgatgaaataagaaaaaatgcac |
| | | agctgaatattgaactggaagcagcacatcatta |
| SEQ ID NO: 7 | Mouse cDNA | agctatgttgtaaccaatggccttggggttaatgtgtttaa |
| | encoding NAMPT cif | ggacccagttgctgatcccaacaaaaggtcaaaaaagg |
| | fragment | gccggttatctttacataggacaccagcggggaactttgt |
| | | tacacttgaagaaggaaaaggagaccttgaggaatatg |
| | | gccatgatcttctccatacggttttcaagaatgggaaggt |
| | | gacaaaaagctactcatttgatgaagtcagaaaaaatgc |
| | | acagctgaacatcgagcaggacgtggcacctcatt |
| SEQ ID NO: 8 | Example of Human | atgaatcctgcggcagaagccgagttcaacatcctcctg |
| | DNA encoding | gccaccgactcctacaaggttactcactataaacaatatc |
| | NAMPT full length | cacccaacacaagcaaagtttattcctactttgaatgccgt |
| | | gaaaagaagacagaaaactccaaattaaggaaggtgaa |
| | | atatgaggaaacagtattttatgggttgcagtacattcttaa |
| | | taagtacttaaaaggtaaagtagtaaccaaagagaaaat |
| | | ccaggaagccaaagatgtctacaaagaacatttccaaga |
| | | tgatgtctttaatgaaaagggatggaactacattcttgaga |
| | | agtatgatgggcatcttccaatagaaataaaagctgttcct |
| | | gagggctttgtcattcccagaggaaatgttctcttcacggt |
| | | ggaaaacacagatccagagtgttactggcttacaaattg |
| | | gattgagactattcttgttcagtcctggtatccaatcacagt |
| | | ggccacaaattctagagagcagaagaaaatattggcca |
| | | aatatttgttagaaacttctggtaacttagatggtctggaat |
| | | acaagttacatgattttggctacagaggagtctcttcccaa |
| | | gagactgctggcataggagcatctgctcacttggttaact |
| | | tcaaaggaacagatacagtagcaggacttgctctaattaa |
| | | aaaatattatggaacgaaagatcctgttccaggctattctg |
| | | ttccagcagcagaacacagtaccataacagcttggggg |
| | | aaagaccatgaaaaagatgcttttgaacatattgtaacac |
| | | agttttcatcagtgcctgtatctgtggtcagcgatagctatg |
| | | acatttataatgcgtgtgagaaaatatggggtgaagatct |
| | | aagacatttaatagtatcgagaagtacacaggcaccact |
| | | aataatcagacctgattctggaaaccctcttgacactgtgt |
| | | taaaggttttggagattttaggtaagaagtttcctgttactg |
| | | agaactcaaagggttacaagttgctgccaccttatcttag |
| | | agttattcaaggggatggagtagatattaataccttacaag |
| | | agattgtagaaggcatgaaacaaaaaatgtggagtattg |
| | | aaaatattgccttcggttctggtggaggtttgctacagaag |
| | | ttgacaagagatctcttgaattgttccttcaagtgtagctat |
| | | gttgtaactaatggccttgggattaacgtcttcaaggaccc |
| | | agttgctgatcccaacaaaaggtccaaaaagggccgatt |
| | | atctttacataggacgccagcagggaattttgttacactgg |
| | | aggaaggaaaaggagaccttgaggaatatggtcaggat |
| | | cttctccatactgtcttcaagaatggcaaggtgacaaaaa |
| | | gctattcatttgatgaaataagaaaaaatgcacagctgaat |
| | | attgaactggaagcagcacatcatta |
| SEQ ID NO: 9 | Mouse cDNA | atgaatgctgcggcagaagccgagttcaacatcctgctg |
| | encoding NAMPT | gccaccgactcgtacaaggttactcactataaacaatacc |
| | full-length | cacccaacacaagcaaagtttattcctactttgaatgccgt |
| | | gaaaagaagacagaaaactccaaagtaaggaaggtga |
| | | aatacgaggaaacagtattttatgggttgcagtacattctt |
| | | aataagtacttaaaaggtaaagtagtgaccaaagagaaa |
| | | atccaggaggccaaagaagtgtacagagaacatttcca |
| | | agatgatgtctttaacgaaagaggatggaactacatcctt |
| | | gagaaatacgatggtcatctcccgattgaagtaaaggct |
| | | gttcccgagggctctgtcatccccagagggaacgtgctg |
| | | ttcacagtggaaaacacagacccagagtgctactggctt |
| | | accaattggattgagactattcttgttcagtcctggtatcca |
| | | attacagtggccacaaattccagagaacagaagagaata |
| | | ctggccaaatatttgttagaaacctctggtaacttagatgg |
| | | tctggaatacaagttacatgactctggttacagaggagtct |
| | | cttcgcaagagactgctggcataggggcatctgctcattt |
| | | ggttaacttaaaaggaacagatactgtgggggaattgct |
| | | ctaattaaaaaatactatgggacaaaagatcctgttccag |
| | | gctattctgttccagcagcagagcacagtaccataacgg |
| | | cttgggggaaagaccatgagaaagatgcttttgaacaca |
| | | tagtaacacagttctcatcagtgcctgtgtctgtggtcagc |
| | | gatagctatgacatttataatgcgtgtgagaaaatatggg |
| | | gtgaagacctgagacatctgatagtatcgagaagtacag |
| | | aggcaccactaatcatcagacctgactctggaaatcctct |
| | | tgacactgtattgaaggtcttagatattttaggcaagaagtt |
| | | tcctgttactgagaactcaaaaggctacaagttgctgcca |
| | | ccttatcttagagtcattcaaggagatggcgtggatatcaa |
| | | tactttacaagagattgtagagggaatgaaacaaaagaa |
| | | gtggagtatcgagaatgtctccttcggttctggtggcgctt |
| | | tgctacagaagttaacccgagacctcttgaattgctccttc |
| | | aagtgcagctatgttgtaaccaatggccttggggttaatgt |
| | | gtttaaggacccagttgctgatcccaacaaaaggtcaaa |
| | | aaagggccggttatctttacataggacaccagcgggga |
| | | actttgttacacttgaagaaggaaaaggagaccttgagg |
| | | aatatggccatgatcttctccatacggttttcaagaatggg |
| | | aaggtgacaaaaagctactcatttgatgaagtcagaaaa |
| | | aatgcacagctgaacatcgagcaggacgtggcacctca |
| | | tt |
| SEQ ID NO: 10 | DNA Primer | 5′-GAGtatttaggtgacactatagGGTTTCATC |
| | | GCAAGAGACGG-3′ |
| SEQ ID NO: 11 | DNA Primer | 5′-GAGtaatacgactcactatagggGCGGAAG |
| | | CACCTTATAGCCT-3′ |
| SEQ ID NO: 12 | DNA Primer | 5′-TTATAACCAAGAGACATGTCGGC |
| | | G-3′ |
| SEQ ID NO: 13 | DNA Primer | 5′-ACCCAGACGACCAGACCATT-3′ |

Supplementary Table 2

| | | | | Percentage of | | | | | |
|---|---|---|---|---|---|---|---|---|---|
| | | | Frequency of | macrophages | | | | | |
| | | Number of | macrophages | from each | | Percentage | | Percentage | Percentage |
| | | macrophages | from each | isolation time | Total | of total | Percentage | of transient | of dwelling |
| | Isolation | from each | isolation time | point in | number of | macrophages | of uninjured | macrophages | macrophages |
| | time | isolation time | point (relative | individual | macrophages | in individual | macrophages | (1 dpi) | (2 and 3 dpi) |
| Cluster | point | point | to cluster) | clusters | in cluster | clusters | in cluster | in cluster | in cluster |
| 0 | Uninjured | 2 | 0.54% | 1.00% | 371 | 28% | 0.54% | 8.36% | 91.11% |
| | 1 dpi | 31 | 8.36% | 7.75% | | | | | |
| | 2 dpi | 116 | 31.27% | 51.33% | | | | | |
| | 3 dpi | 222 | 59.84% | 46.06% | | | | | |
| 1 | Uninjured | 1 | 0.29% | 0.50% | 348 | 27% | 0.29% | 90.52% | 9.20% |
| | 1 dpi | 315 | 90.52% | 78.75% | | | | | |
| | 2 dpi | 19 | 5.46% | 8.41% | | | | | |
| | 3 dpi | 13 | 3.74% | 2.70% | | | | | |
| 2 | Uninjured | 3 | 1.36% | 1.49% | 221 | 17% | 1.36% | 18.10% | 80.54% |
| | 1 dpi | 40 | 18.10% | 10.00% | | | | | |
| | 2 dpi | 59 | 26.70% | 26.11% | | | | | |
| | 3 dpi | 119 | 53.85% | 24.69% | | | | | |
| 3 | Uninjured | 169 | 92.86% | 84.08% | 182 | 14% | 92.86% | 0.00% | 7.14% |
| | 3 dpi | 13 | 7.14% | 2.70% | | | | | |
| | 1 dpi | 3 | 4.35% | 0.75% | | | | | |
| | 2 dpi | 10 | 14.49% | 4.42% | | | | | |
| | 3 dpi | 44 | 63.77% | 9.13% | | | | | |
| 5 | Uninjured | 2 | 4.08% | 1.00% | 49 | 4% | 4.08% | 10.20% | 85.71% |
| | 1 dpi | 5 | 10.20% | 1.25% | | | | | |
| | 2 dpi | 11 | 22.45% | 4.87% | | | | | |
| | 3 dpi | 31 | 63.27% | 6.43% | | | | | |
| 6 | Uninjured | 8 | 16.67% | 3.98% | 48 | 4% | 16.67% | 10.42% | 72.92% |
| | 1 dpi | 5 | 10.42% | 1.25% | | | | | |
| | 2 dpi | 2 | 4.17% | 0.88% | | | | | |
| | 3 dpi | 33 | 68.75% | 6.85% | | | | | |
| 7 | Uninjured | 4 | 19.05% | 1.99% | 21 | 2% | 19.05% | 4.76% | 76.19% |
| | 1 dpi | 1 | 4.76% | 0.25% | | | | | |
| | 2 dpi | 9 | 42.86% | 3.98% | | | | | |
| | 3 dpi | 7 | 33.33% | 1.45% | | | | | |

| | |
|---|---|
| Total number of macrophages | 1309 |
| Total number of uninjured macrophages | 201 |
| Total number of 1 dpi macrophages | 400 |
| Total number of 2 dpi macrophages | 226 |
| Total number of 3 dpi macrophages | 482 |
| Total number of dwelling macrophages (2 and 3 dpi) | 708 |

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety. The full contents of Australian provisional application 2020904717 filed 17 Dec. 2020 and Australian provisional 2020901237 filed 20 Apr. 2020 are incorporated by reference herein in their entirety. The Figures are also clearly represented in colour in the inventor publication in Nature 2021 March; 591(7849):281-287. doi: 10.1038/s41586-021-03199-7. Epub 2021 Feb. 10.

Those of skill in the art will appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Gurevich, D. B. et al. Asymmetric division of clonal muscle stem cells co-ordinate muscle regeneration in vivo. Science (2016).
Novak, M. L. & Koh, T. J. Macrophage phenotypes during tissue repair. Journal of leukocyte biology 93, 875-881 (2013).
Yahiaoui, L., Gvozdic, D., Danialou, G., Mack, M. & Petrof, B. J. CC family chemokines directly regulate myoblast responses to skeletal muscle injury. The Journal of physiology 586, 3991-4004 (2008).
Grolla, A. A., Travelli, C., Genazzani, A. A. & Sethi, J. K. Extracellular nicotinamide phosphoribosyltransferase, a new cancer metabokine. British journal of pharmacology 173, 2182-2194 (2016).
Van den Bergh, R. et al. Monocytes contribute to differential immune pressure on R5 versus X4 HIV through the adipocytokine visfatin/NAMPT. PloS one 7, e35074 (2012).
Quarta, M. et al. Bioengineered constructs combined with exercise enhance stem cell-mediated treatment of volumetric muscle loss. Nature communications 8, 1-17 (2017).
Mochizuki et al., Nat. Biomed Engineering 2019.

1. A method of stimulating muscle tissue regeneration, or treating a muscular, neuromuscular, or musculoskeletal deficiency, disorder or injury, the method comprising administering to a subject an effective amount of a composition comprising or encoding a CCR5 interacting agent, wherein the CCR5 agonist binds to muscle stem cells and stimulates myoblast proliferation and muscle regeneration.

2.-4. (canceled)

5. The method of claim 1, wherein the tissue regeneration occurs without or substantially without fibrosis.

6.-8. (canceled)

9. The method of claim 1, wherein the composition is a cellular composition comprising a cell that expresses the CCR5 agonist.

10. The method of claim 9, wherein the cell is a macrophage.

11. (canceled)

12. The method of claim 1, wherein the CCR5 agonist in the composition is NAMPT or a part thereof comprising a cytokine finger (cif) motif or a derivative thereof.

13.-26. (canceled)

27. A NAMPT polypeptide fragment comprising a C-terminal portion of NAMPT comprising a cytokine finger (cif) motif, or a modified form thereof, or a functional derivative thereof.

28. The NAMPT polypeptide fragment or modified form or functional derivative thereof of claim 27 comprising the peptide sequence set forth in SEQ ID NO: 1 or 2 or 5 or a sequence having at least 80% identity to SEQ ID NO: 1 or 2 or 5.

29. The NAMPT fragment or modified form or functional derivative thereof of claim 27, wherein the fragment is in monomeric, dimeric or multimeric form.

30.-49. (canceled)

50. The method of claim 1, wherein the composition is administered to a muscle of the subject.

51. The method of claim 1, wherein the composition further comprises a component that enhances delivery to the muscle.

52. The method of claim 1, wherein the CCR5 agonist in the composition is a NAMPT polypeptide fragment comprising a C-terminal portion of NAMPT comprising a cytokine finger (cif) motif, or a modified form thereof, or a functional derivative thereof.

53. The method of claim 1, wherein the CCR5 agonist comprises or consists of the peptide sequence set forth in SEQ ID NO: 3.

54. The NAMPT polypeptide fragment or modified form or functional derivative thereof of claim 27, having 1, 2, 3, 4, 5 or 6 conservative or non-conservative amino acid substitutions, deletions or additions, and retains CCR5 agonist activity.

55. The NAMPT polypeptide fragment or modified form or functional derivative thereof of claim 27, comprising one or more of: a linker; a stability enhancing moiety; a signalling enhancing moiety, preferably a heparin sulphate binding moiety such as a syndecan binding moiety; a delivery enhancing moiety; a tissue or muscle retention enhancing moiety; and a label moiety.

56. A composition comprising or encoding the NAMPT fragment or modified form or functional derivative thereof of claim 27.

57. The composition of claim 56, wherein the composition further comprises a tissue stem cell or precursor therefore or progeny thereof.

58. The composition of claim 56, wherein the composition further comprises a macrophage or a precursor therefore or progeny thereof.

59. The composition of claim 56, wherein the composition further comprises a scaffold (semi-solid or solid support) or a retentive material.

60. The composition of claim 59, wherein the scaffold or retentive material is a hydrogel, such as a fibrin or acrylamide hydrogel.

61. The composition of claim 56, wherein the composition further comprises a tissue delivery enhancing or cell retention moiety, preferably an ECM-binding moiety.


SEQUENCE LISTING (13 sequences)

SEQ ID NO: 1 - protein, 90 aa, Homo sapiens
SYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 2 - protein, 90 aa, Mus musculus
SYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 3 - protein, 491 aa, Homo sapiens [sequence omitted]

SEQ ID NO: 4 - protein, 491 aa, Mus musculus
MNAAAEAEFNILLATDSYKVTHYKQYPPNTSKVYSYFECREKKTENSKVRKVKYEETVFYGLQYILNKYLKGKVVTKEKIQEAKEVYREHFQDDVFNERGWNYILEKYDGHLPIEVKAVPEGSVIPRGNVLFTVENTDPECYWLTNWIETILVQSWYPITVATNSREQKKILAKYLLETSGNLDGLEYKLHDFGYRGVSSQETAGIGASAHLVNFKGTDTVAGIALIKKYYGTKDPVPGYSVPAAEHSTITAWGKDHEKDAFEHIVTQFSSVPVSVVSDSYDIYNACEKIWGEDLRHLIVSRSTEAPLIIRPDSGNPLDTVLKVLDILGKKFPVTENSKGYKLLPPYLRVIQGDGVDINTLQEIVEGMKQKKWSIENVSFGSGGALLQKLTRDLLNCSFKCSYVVTNGLGVNVFKDPVADPNKRSKKGRLSLHRTPAGNFVTLEEGKGDLEEYGHDLLHTVFKNGKVTKSYSFDEVRKNAQLNIEQDVAPH

SEQ ID NO: 5 - protein, 91 aa, Homo sapiens
CSYVVTNGLGINVFKDPVADPNKRSKKGRLSLHRTPAGNFVTLEEGKGDLEEYGQDLLHTVFKNGKVTKSYSFDEIRKNAQLNIELEAAHH

SEQ ID NO: 6 - DNA, 272 bp, Homo sapiens
agctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcatta

SEQ ID NO: 7 - DNA, 271 bp, Mus musculus
agctatgttgtaaccaatggccttggggttaatgtgtttaaggacccagttgctgatcccaacaaaaggtcaaaaaagggccggttatctttacataggacaccagcggggaactttgttacacttgaagaaggaaaaggagaccttgaggaatatggccatgatcttctccatacggttttcaagaatgggaaggtgacaaaaagctactcatttgatgaagtcagaaaaaatgcacagctgaacatcgagcaggacgtggcacctcatt

SEQ ID NO: 8 - DNA, 1475 bp, Homo sapiens
atgaatcctgcggcagaagccgagttcaacatcctcctggccaccgactcctacaaggttactcactataaacaatatccacccaacacaagcaaagtttattcctactttgaatgccgtgaaaagaagacagaaaactccaaattaaggaaggtgaaatatgaggaaacagtattttatgggttgcagtacattcttaataagtacttaaaaggtaaagtagtaaccaaagagaaaatccaggaagccaaagatgtctacaaagaacatttccaagatgatgtctttaatgaaaagggatggaactacattcttgagaagtatgatgggcatcttccaatagaaataaaagctgttcctgagggctttgtcattcccagaggaaatgttctcttcacggtggaaaacacagatccagagtgttactggcttacaaattggattgagactattcttgttcagtcctggtatccaatcacagtggccacaaattctagagagcagaagaaaatattggccaaatatttgttagaaacttctggtaacttagatggtctggaatacaagttacatgattttggctacagaggagtctcttcccaagagactgctggcataggagcatctgctcacttggttaacttcaaaggaacagatacagtagcaggacttgctctaattaaaaaatattatggaacgaaagatcctgttccaggctattctgttccagcagcagaacacagtaccataacagcttgggggaaagaccatgaaaaagatgcttttgaacatattgtaacacagttttcatcagtgcctgtatctgtggtcagcgatagctatgacatttataatgcgtgtgagaaaatatggggtgaagatctaagacatttaatagtatcgagaagtacacaggcaccactaataatcagacctgattctggaaaccctcttgacactgtgttaaaggttttggagattttaggtaagaagtttcctgttactgagaactcaaagggttacaagttgctgccaccttatcttagagttattcaaggggatggagtagatattaataccttacaagagattgtagaaggcatgaaacaaaaaatgtggagtattgaaaatattgccttcggttctggtggaggtttgctacagaagttgacaagagatctcttgaattgttccttcaagtgtagctatgttgtaactaatggccttgggattaacgtcttcaaggacccagttgctgatcccaacaaaaggtccaaaaagggccgattatctttacataggacgccagcagggaattttgttacactggaggaaggaaaaggagaccttgaggaatatggtcaggatcttctccatactgtcttcaagaatggcaaggtgacaaaaagctattcatttgatgaaataagaaaaaatgcacagctgaatattgaactggaagcagcacatcatta

SEQ ID NO: 9 - DNA, 1474 bp, Mus musculus
atgaatgctgcggcagaagccgagttcaacatcctgctggccaccgactcgtacaaggttactcactataaacaatacccacccaacacaagcaaagtttattcctactttgaatgccgtgaaaagaagacagaaaactccaaagtaaggaaggtgaaatacgaggaaacagtattttatgggttgcagtacattcttaataagtacttaaaaggtaaagtagtgaccaaagagaaaatccaggaggccaaagaagtgtacagagaacatttccaagatgatgtctttaacgaaagaggatggaactacatccttgagaaatacgatggtcatctcccgattgaagtaaaggctgttcccgagggctctgtcatccccagagggaacgtgctgttcacagtggaaaacacagacccagagtgctactggcttaccaattggattgagactattcttgttcagtcctggtatccaattacagtggccacaaattccagagaacagaagagaatactggccaaatatttgttagaaacctctggtaacttagatggtctggaatacaagttacatgactctggttacagaggagtctcttcgcaagagactgctggcataggggcatctgctcatttggttaacttaaaaggaacagatactgtggcgggaattgctctaattaaaaaatactatgggacaaaagatcctgttccaggctattctgttccagcagcagagcacagtaccataacggcttgggggaaagaccatgagaaagatgcttttgaacacatagtaacacagttctcatcagtgcctgtgtctgtggtcagcgatagctatgacatttataatgcgtgtgagaaaatatggggtgaagacctgagacatctgatagtatcgagaagtacagaggcaccactaatcatcagacctgactctggaaatcctcttgacactgtattgaaggtcttagatattttaggcaagaagtttcctgttactgagaactcaaaaggctacaagttgctgccaccttatcttagagtcattcaaggagatggcgtggatatcaatactttacaagagattgtagagggaatgaaacaaaagaagtggagtatcgagaatgtctccttcggttctggtggcgctttgctacagaagttaacccgagacctcttgaattgctccttcaagtgcagctatgttgtaaccaatggccttggggttaatgtgtttaaggacccagttgctgatcccaacaaaaggtcaaaaaagggccggttatctttacataggacaccagcggggaactttgttacacttgaagaaggaaaaggagaccttgaggaatatggccatgatcttctccatacggttttcaagaatgggaaggtgacaaaaagctactcatttgatgaagtcagaaaaaatgcacagctgaacatcgagcaggacgtggcacctcatt

SEQ ID NO: 10 - DNA, 42 bp, Artificial Sequence
  Synthetic primer
gagtatttaggtgacactatagggtttcatcgcaagagacgg

SEQ ID NO: 11 - DNA, 43 bp, Artificial Sequence
  Synthetic primer
gagtaatacgactcactataggggcggaagcaccttatagcct

SEQ ID NO: 12 - DNA, 24 bp, Artificial Sequence
  Synthetic primer
ttataaccaagagacatgtcggcg

SEQ ID NO: 13 - DNA, 20 bp, Artificial Sequence
  Synthetic primer
acccagacgaccagaccatt